(19) United States
(12) Patent Application Publication
(10) Pub. No.: US 2020/0384003 A1
(43) Pub. Date: Dec. 10, 2020

(54) TRITERPENOID-CONTAINING PHARMACEUTICAL COMPOSITION AND USE THEREOF

(71) Applicants: Qinyuan ZHENG, Shanghai (CN); SHANGHAI LANMU CHEMICALS CO., LTD., Shanghai (CN)

(72) Inventors: Qinyuan ZHENG, Shanghai (CN); Jingjie ZHU, Shanghai (CN); Jing QU, Shanghai (CN); Yu LUO, Shanghai (CN); Shicheng DING, Shanghai (CN); Lijun QIN, Shanghai (CN); Xiangyan ZHOU, Shanghai (CN); Chuanmin SUN, Shanghai (CN); Fenqin ZHAO, Shanghai (CN); Cunqing ZHANG, Shanghai (CN); Zhongwei JI, Shanghai (CN); Li GUO, Shanghai (CN)

(21) Appl. No.: 16/959,084

(22) PCT Filed: Dec. 28, 2018
(86) PCT No.: PCT/CN2018/125075

§ 371 Date: Jun. 29, 2020

(30) Foreign Application Priority Data
Dec. 29, 2017 (CN) 201711480630.3
Dec. 29, 2017 (CN) 201711480659.1
Dec. 29, 2017 (CN) 201711484482.2
Dec. 29, 2017 (CN) 201711489391.8

(51) Int. Cl. A61K 31/704; A61K 31/575

(52) U.S. Cl. CPC A61K 31/704; A61K 9/0051; A61K 31/575

(57) ABSTRACT

The present invention relates to a novel use of a triterpenoid. In particular, the present invention provides a use of a compound of formula I, or an optical isomer thereof, or a racemate thereof, or a solvate thereof, or a pharmaceutically acceptable salt thereof, or a prodrug thereof in the preparation of a pharmaceutical composition or a preparation for preventing and/or treating ocular diseases caused by crystalline lens lesions. A novel finding of the present invention is that the compound of formula I can prevent and/or treat ocular diseases caused by crystalline lens lesions, such as prevention and treatment of cataracts, is safe, has low toxicity and few side effects, and has remarkable therapeutic efficacy and development and application prospects.

### FIELD OF THE INVENTION

The present invention relates to the field of medicine. Specifically, the present invention relates to a novel use of a triterpenoid or inonotus obliquus extract.

### BACKGROUND OF THE INVENTION

The lens of a normal human eye is composed of a plurality of ordered lens proteins. Once these lens proteins are arranged incorrectly or misfolded, the protein aggregates are formed, which affects the normal transparency and refractive index of the lens. Wherein, cataract is a major form of manifestation, and it is also the world's highest blinding disease. Therefore, the treatment and prevention of lens diseases, especially cataracts, is important. Unfortunately, the surgery and artificial intraocular lens replacement are still the main cataract treatment means in clinic.

How to use drugs simply to prevent, inhibit the development of the disease and even treat cataracts has always been a hot spot in ophthalmology research. Previous studies have reported that lanosterol can be used to treat cataracts in animals. The structural formula of lanosterol is as follows:

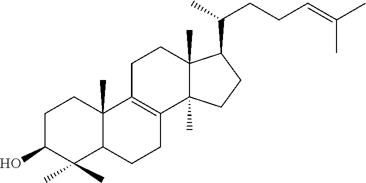

However, it is worth noting that the sustained-release lanosterol drug has a cataract treatment effect on dogs only when combined with high-frequency intravitreal injection. Moreover, different researchers have also pointed out that the direct administration of lanosterol to the eyeball or lens does not restore or improve the transparency of cataract lens in primates.

Therefore, there is an urgent need in the art to develop a medicament that can effectively treat lens lesions or eye diseases related to lens lesions, In particular, the medicament have satisfactory results in primate pharmacodynamics tests.

### SUMMARY OF THE INVENTION

It is an object of the present invention to provide a medicament that can effectively treat lens lesions or eye diseases related to lens lesions and use thereof.

In the first aspect of the present invention, a use of a compound of formula I, or an optical isomer, a racemate, a solvate, a pharmaceutically acceptable salt, a prodrug, or a deuterated compound thereof is provided for the manufacture of a pharmaceutical composition or formulation, the pharmaceutical composition or formulation are used to prevent and/or treat eye diseases caused by lens lesions;

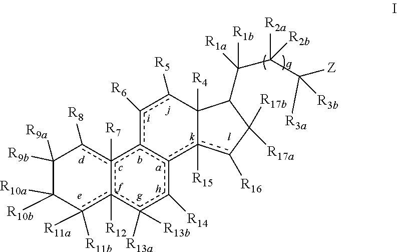

wherein,

q is 0, 1 or 2;

R1a, R1b, R2a, R2b, R3a and R3b are each independently selected from hydrogen, substituted or unsubstituted C1-C20 alkyl, substituted or unsubstituted C2-C20 alkenyl, substituted or unsubstituted C2-C20 alkynyl, substituted or unsubstituted C3-C10 cycloalkyl, —OH, substituted or unsubstituted C1-C10 alkoxy, —COOH, —CHO, substituted or unsubstituted C1-C10 ester group, —SH, substituted or unsubstituted C1-C10 alkylthio, -A-B,

or R1a and R1b, R2a and R2b, and/or R3a and R3b constitute ═O;

wherein, A is absent or a divalent linking group; and B is H, —OH, —SH, C1-C3 alkoxy, C1-C3 alkylthio, —CHO, —COOH, C1-C4 ester group, C3-C10 cycloalkyl, aryl, C3-C10 5-6 membered heteroaryl, or benzyl;

with the proviso that at least one of R1a, R2a, R3a, R1b, R2b and R3b is a group containing O or S;

Z is selected from the group consisting of H, substituted or unsubstituted C1-C20 alkyl, substituted or unsubstituted C2-C20 alkenyl, substituted or unsubstituted C2-C20 alkynyl, substituted or unsubstituted C3-C10 cycloalkyl, substituted or unsubstituted aryl, substituted or unsubstituted 5-8 membered heteroaryl, substituted or unsubstituted —(C1-C6 alkylene)-aryl, substituted or unsubstituted-(C1-C6 alkylene)-5-8 membered heteroaryl, —OH, substituted or unsubstituted C1-C20 alkoxy, —SH, substituted or unsubstituted C1-C20 alkylthio, substituted or unsubstituted C1-C10 ester group, substituted or unsubstituted C1-C10 acyl, and substituted or unsubstituted —O-aryl;

R4 is hydrogen, substituted or unsubstituted C1-C4 alkyl;

R7, R12 and R15 are each independently selected from absent, hydrogen, substituted or unsubstituted C1-C4 alkyl;

R13a and R13b are each independently selected from absent, hydrogen, substituted or unsubstituted C1-C8 alkyl, —OH, substituted or unsubstituted C1-C8 alkoxy, —SH, substituted or unsubstituted C1-C8 alkylthio, halogen, substituted or unsubstituted C1-C3 acyl, or R13a and R13b constitute ═O, and at most one of R13a and R13b is absent;

R11a and R11b are each independently selected from absent, hydrogen, substituted or unsubstituted C1-C6 alkyl, —OH, or R11a and R11b constitute ═O, and at most one of R11a and R11b is absent;

R10a and R10b are each independently selected from hydrogen, —OH, substituted or unsubstituted C1-C8 alkoxy, —SH, substituted or unsubstituted C1-C8 alkylthio, —OSO₃H, —OCO-substituted or unsubstituted C1-C7 alkyl, —OPO₃H, —COOH, -(substituted or unsubstituted C1-C7 alkylene)-COOH, —CHO, -(substituted or unsubstituted C1-C7 alkylene)-CHO, or R10a and R10b together constitute ═O; and

R5, R6, R8, R9a, R9b, R14, R16, R17a and R17b are each independently selected from the group consisting of hydrogen, OH, substituted or unsubstituted C1-C8 alkyl, substituted or unsubstituted C1-C8 alkoxy, and halogen.

In another preferred embodiment, the “substituted” means that one or more hydrogen atoms on the group are substituted by group(s) selected from the group consisting of halogen, —OH, —SH, —COOH, —(C1-C7 alkylene)-COOH, ═O, —CHO, —(C1-C7 alkylene)-CHO, C1-C7 alkyl-OCO—, C1-C3 alkyl, C3-C6 cycloalkyl, cyano, nitro, cyanate group, isocyanate group, isothiocyanate group, sulfonamido, oximido, NRaRb, wherein, Ra and Rb are each independently selected from H, C1-C6 alkyl, C1-C6 haloalkyl, C3-C6 cycloalkyl, or benzyl.

In another preferred embodiment, the “substituted” means that one or more hydrogen atoms on the group are substituted by group(s) selected from the group consisting of halogen, —OH, —SH, —COOH, —(C1-C7 alkylene)-COOH, ═O, —CHO, —(C1-C7 alkylene)-CHO, C1-C7 alkyl-OCO—, C1-C3 alkyl, C3-C6 cycloalkyl, NRaRb, wherein Ra and Rb is each independently H, C1-C6 alkyl, C1-C6 haloalkyl, C3-C6 cycloalkyl, or benzyl.

In another preferred embodiment, the total number of double bonds represented by the dotted line ( [symbol] ) in the structural formula is 0, 1, 2, 3 or 4.

In another preferred embodiment, the double bond is located between the following positions: a and b, b and c, c and d, e and f, c and f, f and g, g and h, h and a, b and i, i and j, a and k, and/or k and l.

In another preferred embodiment, the double bond is located between the following positions: a and b.

In another preferred embodiment, the double bond is located between the following positions: h and a, and/or b and i.

In another preferred embodiment, the valence state of each C conforms to the requirements of the chemically stable structure (ie, C is tetravalent) in the structural formula.

When R5, R6, R7, R8, R12, R14, R15 and/or R16 are bonded to C containing a double bond, the valence of the C is tetravalent.

In another preferred embodiment, the divalent linking group has 1-10 linking units selected from the group consisting of —CRaRb—, —C(OH)Ra—, —NRa—, —O—, and —CO—, wherein, Ra and Rb are each independently selected from H, C1-C6 alkyl, C1-C6 haloalkyl, C3-C6 cycloalkyl, or benzyl.

In another preferred embodiment, R1a, R1b, R2a, R2b, R3a and R3b are each independently selected from hydrogen, —OH, —CHO, —COOH, —SH, substituted or unsubstituted C1-C3 alkyl-OCO—, substituted or unsubstituted C1-C4 alkyl, -A-B, or R1a and R1b, R2a and R2b, and/or R3a and R3b constitute ═O;

wherein, A is independently a substituted or unsubstituted C1-C4 alkylene, and B is independently —OH, —SH, C1-C3 alkoxy, —CHO, —COOH, ═O.

In another preferred embodiment, Z is (L1)m-L2=Y, m is 0, 1, 2, 3 or 4, and each L1 is independently —CH₂—, —CO—, —O—, —C(C1-C3 alkyl)H—, C(C1-C3 alkyl)2-, —C(C1-C3 alkyl)O—, or —NH—, L2 is —CH═ or —C(C1-C3 alkyl)=;

Y is ═CH₂, ═CH-substituted or unsubstituted C1-C7 alkyl, ═C-(substituted or unsubstituted C1-C7 alkyl)₂, ═CH-substituted or unsubstituted C2-C7 alkenyl, or ═CH-substituted or unsubstituted C2-C7 alkynyl.

In another preferred embodiment, for Y, the “substituted” means that one or more hydrogen atoms on the group are substituted by group(s) selected from the group consisting of halogen, —OH, —SH, —COOH, —(C1-C7 alkylene)-COOH, ═O, —CHO, —(C1-C7 alkylene)-CHO, C1-C7 alkyl-OCO—, C1-C3 alkyl, C3-C6 cycloalkyl, and NRaRb, wherein Ra and Rb are each independently selected from H, C1-C6 alkyl, C1-C6 haloalkyl, C3-C6 cycloalkyl, or benzyl.

In another preferred embodiment, Y is a group having a hydroxyl group or SH substituent.

In another preferred embodiment, R4 is hydrogen or methyl.

In another preferred embodiment, R7, R12 and R15 are each independently selected from absent, hydrogen, methyl.

In another preferred embodiment, R13a and R13b are each independently selected from hydrogen, methyl, —OH, —SH, ═O, halogen, and at most one of R13a and R13b is absent.

In another preferred embodiment, at least one of R13a and R13b is —OH, or —SH. In another preferred embodiment, R10a and R10b are each independently selected from hydrogen, —OH, —SH, —OSO₃H, —OPO₃H, —COOH, —CHO; or R10a and R10b constitute a carbonyl.

In another preferred embodiment, R5, R6, R8, R9a, R9b, R14, R16, R17a and R17b are each independently selected from hydrogen, methyl.

In another preferred embodiment, R9a, R9b are each independently selected from hydrogen, methyl, halogen.

In another preferred embodiment, R9a is halogen (F, Cl, or Br) and R9b is hydrogen or methyl.

In another preferred embodiment, at least one of R1a and Rib is a group containing O or S.

In another preferred embodiment, at least one of R2a and R2b is a group containing O or S.

In another preferred embodiment, at least one of R3a and R3b is a group containing O or S.

In another preferred embodiment, Z is not —CH₂C(CH₃)₃;

In another preferred embodiment, Z is —CH₂C(CH₃)₂—OH.

In another preferred embodiment, q is 1 or 2.

In another preferred embodiment, the compound of formula I is a compound of formula I-1:

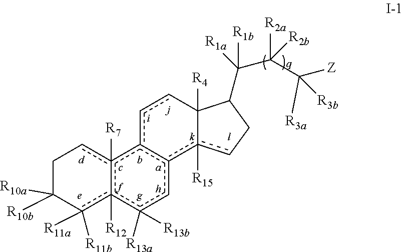

wherein, q, R1a, R1b, R2a, R2b, R3a, R3b, R4, R7, R10a, R10b, R11a, R11b, R12, R13a, R13b, R15 and Z are as defined above.

In another preferred embodiment, R11a and/or R11b are methyl.

In another preferred embodiment, the compound of formula I is a compound of formula I-2:

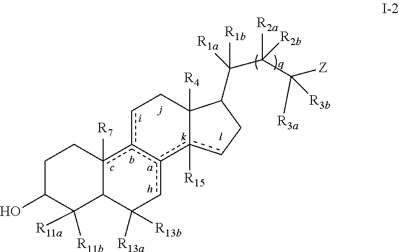

wherein q, R1a, R1b, R2a, R2b, R3a, R3b, R4, R7, R1a, R1b, R13a, R13b, R15 and Z are as defined above.

In another preferred embodiment, a double bond (=) is located between a and b; single bonds are located between the following positions: b and c, a and h, b and i, a and k, and k and i.

In another preferred embodiment, doubles bonds are located between the following positions: b and i, and a and h; single bonds are located between the following positions: a and b, b and c, a and k, and k and i.

In another preferred embodiment, the compound of formula I is selected from the group consisting of:

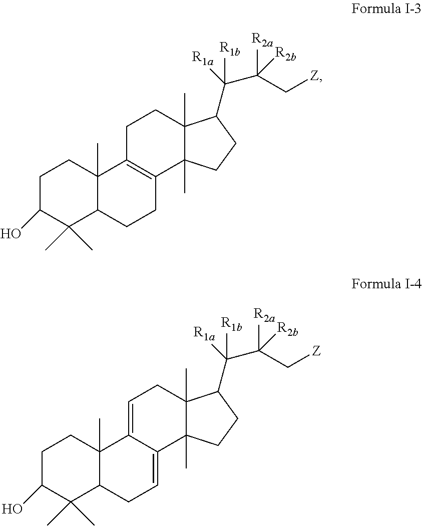

wherein R1a, R1b, R2a, R2b and Z are as defined above.

In another preferred embodiment, the compound of formula I is selected from the group consisting of:

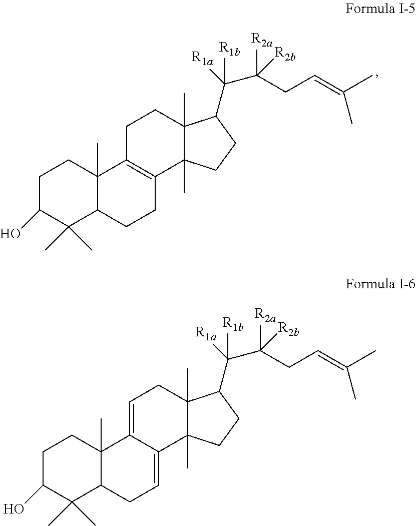

wherein R1a, R1b, R2a, and R2b are as defined above.

In another preferred embodiment, the compound of formula I is a compound of formula I-7:

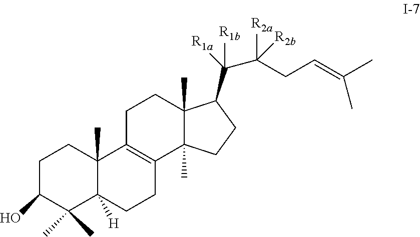

wherein R1a, R1b, R2a, and R2b are as defined above.

In another preferred embodiment, the compound of formula I is a compound of formula I-8:

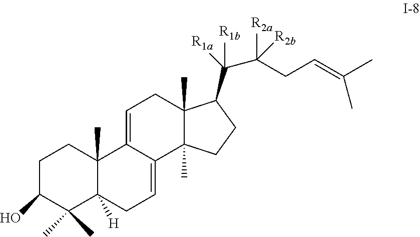

wherein R1a, R1b, R2a, and R2 b are as defined above.

In another preferred embodiment, the compound of formula I is selected from the group consisting of (table A):

TABLE A

| Compound no. | Structural formula |
|---|---|
| 15′ | 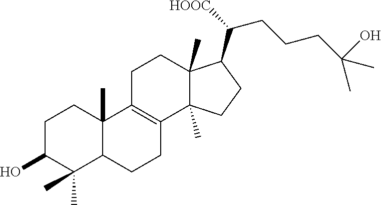 |
| 20′ | 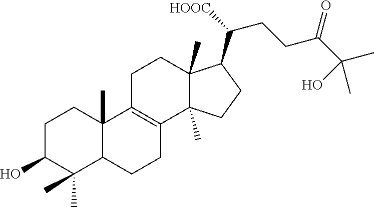 |
| 21′ | 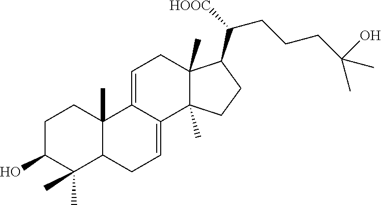 |
| 1 | 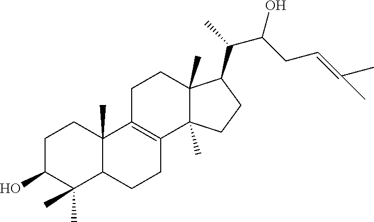 |
| 5 | 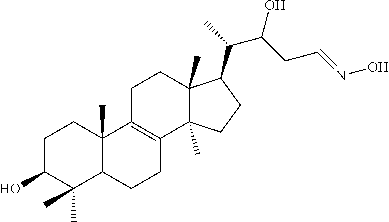 |
| 9 | 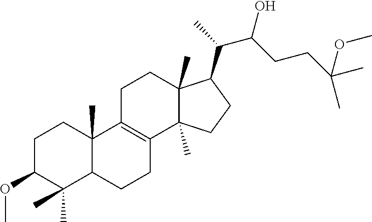 |
| 13 | 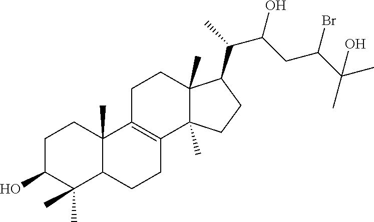 |
| 15 | 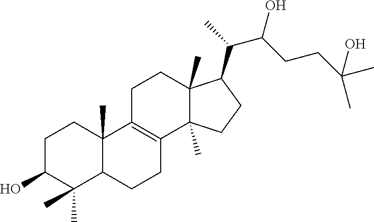 |
| 17 | 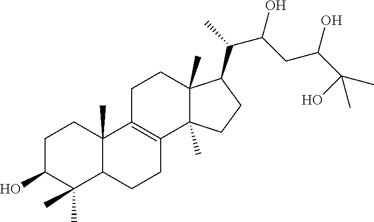 |
| 20 | 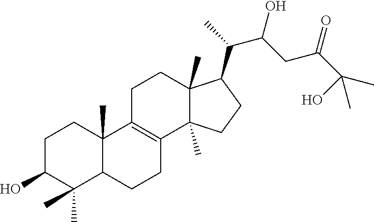 |
| 21 | 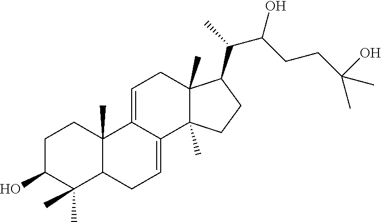 |
| 23 | 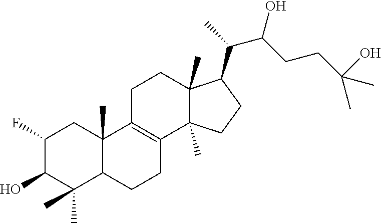 |
| 24 | 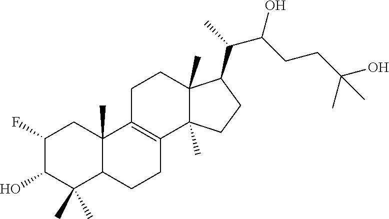 |
| 25 | 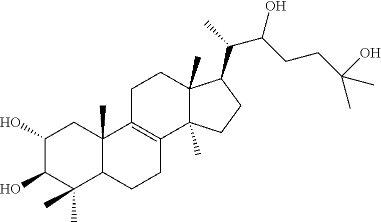 |
| 27 | 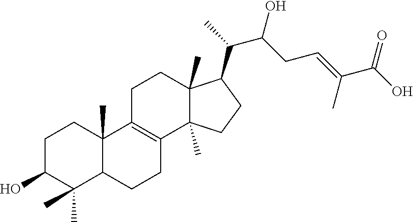 |
| 32 | 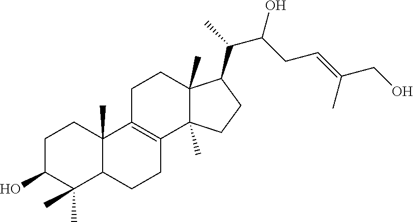 |
| 34 | 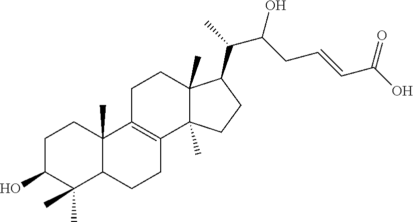 |
| 42 | 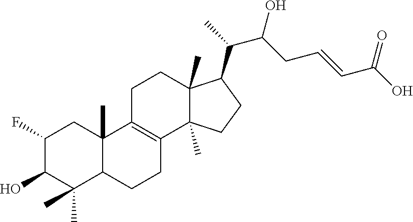 |
| 1″ | 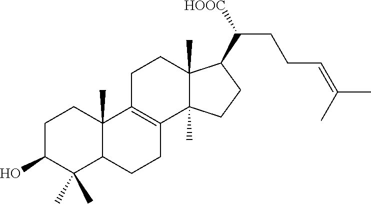 |
| 2″ | 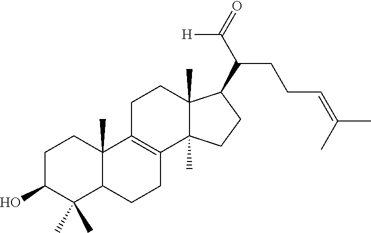 |
| 3″ | 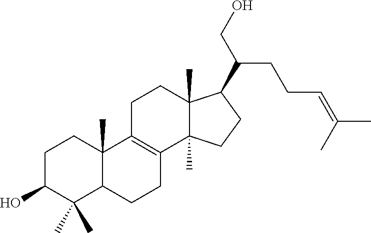 |
| 4″ | 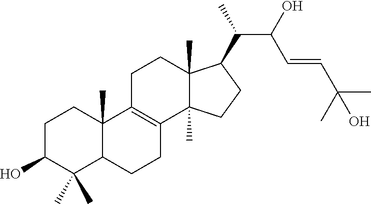 |
| 5″ | 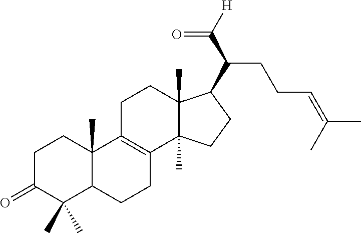 |
| 6″ | 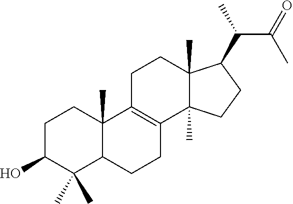 |
| 7″ | 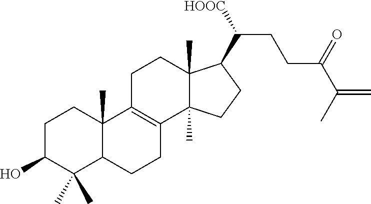 |
| 8″ | 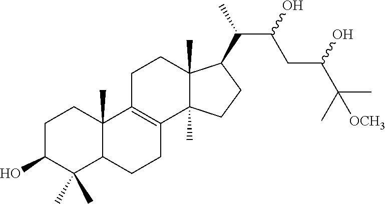 |
| 17″ | 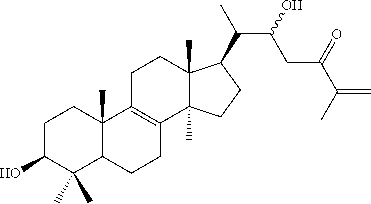 |
| 11 | 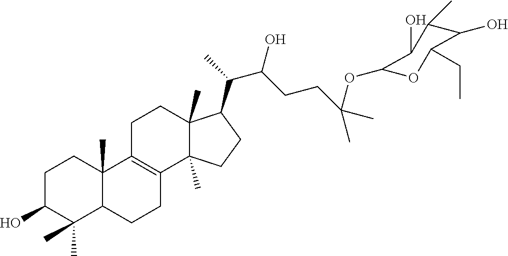 |
| 14 | 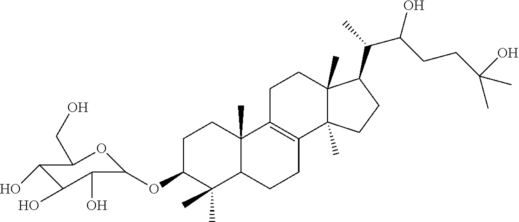 |
| 43 | 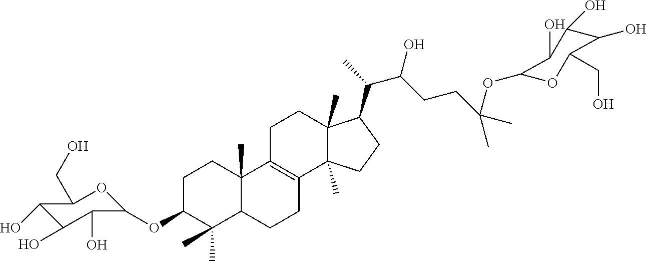 |
| 11′ | 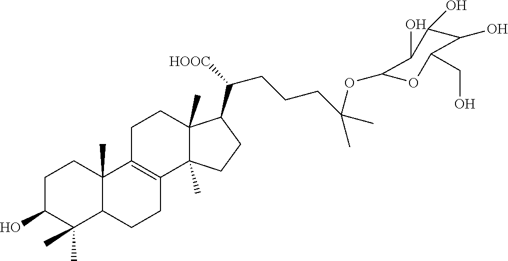 |
| 14′ | 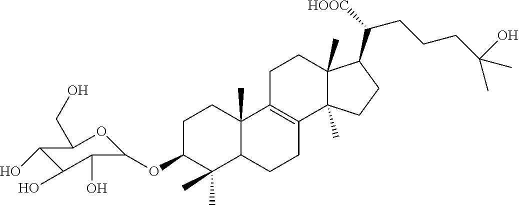 |
| 43′ | 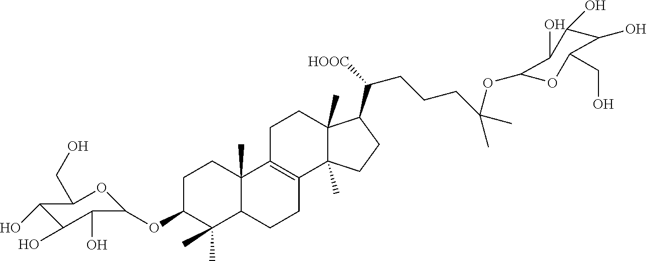 |

In another preferred embodiment, the compound of formula I is selected from the group consisting of:

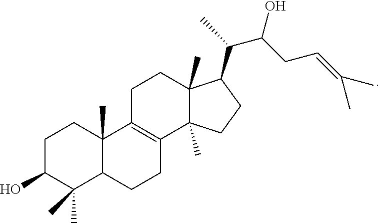

In another preferred embodiment, the compound of formula I is selected from the group consisting of:

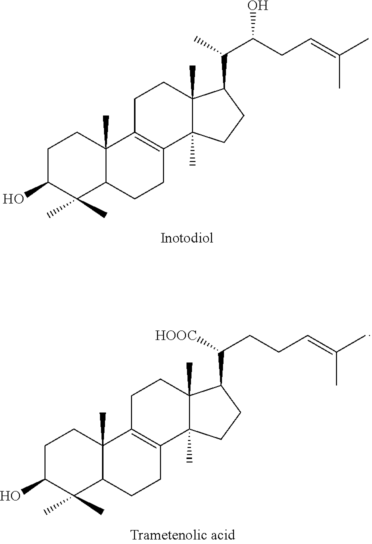

In another preferred embodiment, the eye diseases caused by lens lesions are selected from the following group consisting of cataract, presbyopia, myopia, cortical opacity, presbyopia nuclear sclerosis, and eye complications caused by diabetes.

In another preferred embodiment, the eye diseases are selected from the following group consisting of congenital cataract and acquired cataract.

In another preferred embodiment, the eye diseases are selected from the group consisting of mature cataracts and immature cataracts.

In another preferred embodiment, the cataract is selected from the group consisting of traumatic cataract, metabolic cataract, senile cataract, congenital cataract, spontaneous cataract, complicated cataract, and a combination thereof.

In another preferred embodiment, the metabolic cataract comprises diabetic metabolic cataract.

In another preferred embodiment, the traumatic cataract comprises surgery-related cataract.

In another preferred embodiment, the spontaneous cataract comprise senile spontaneous cataract.

In another preferred embodiment, the pharmaceutical composition or formulation comprises: (a) a therapeutically effective amount of the compound of formula I, or the optical isomer, racemate, solvate, pharmaceutically acceptable salt, or prodrug thereof as a first active ingredient; (b) a pharmaceutically acceptable carrier;

wherein the content of the first active ingredient is 0.001-99 wt %, preferably 0.01-70% wt %, more preferably 0.05-40% wt %, based on the total weight of the composition.

In another preferred embodiment, the pharmaceutical composition or formulation comprises: (a) a therapeutically effective amount of the compound of formula I, or the optical isomer, racemate, solvate, pharmaceutically acceptable salt, prodrug, or deuterated compounds thereof as first active ingredient; (b) a pharmaceutically acceptable carrier;

wherein the content of the first active ingredient is 0.001-99 wt %, preferably 0.01-70 wt %, more preferably 0.05-40 wt % t, based on the total weight of the composition.

In another preferred embodiment, the concentration of the first active ingredient is preferably 1-500 mM or 10-200 mM, preferably 15-150 mM, more preferably 20-50 mM, most preferably 20-30 mM.

In another preferred embodiment, the dosage form of the pharmaceutical composition or formulation is selected from the group consisting of injection, external preparation, and oral preparation.

In another preferred embodiment, the pharmaceutical composition or formulation is an ophthalmic preparation, the ophthalmic preparation is eye drop, emulsion, gel, eye ointment, sustained release microsphere, intraocular sustained-release implant tablet, or medicinal sustained-release film.

In another preferred embodiment, the pharmaceutical composition or formulation further comprises: (c) a second active ingredient, wherein the second active ingredient is selected from the group consisting of lanolin compounds, lanosterols, any compound (especially steroids and terpenoids) contained in fungi in all of Hymenochaetales or Polyporales, azoles, glucocorticoid compounds, antibiotics, or a combination thereof.

In another preferred embodiment, the second active ingredient is selected from the group consisting of lanolin compounds, lanosterols, any compound (especially steroids and terpenoids) contained in fungi in all of Hymenochaetales or Polyporales, azoles, amyloid protein regulator, glucocorticoid compounds, antibiotics, or a combination thereof.

In another preferred embodiment, the content of the second active ingredient is 0.01-20 wt %, preferably 5-15 wt %, based on the total weight of the composition.

In another preferred embodiment, the second active ingredient is lanolin compound or azoles, its concentration is preferably 10-200 mM, more preferably 15-150 mM, more preferably 20-50 mM, most preferably 20-30 mM.

In another preferred embodiment, the lanolin compound is selected from the group consisting of lanosterol, dihydrolanosterol, 25-hydroxycholesterol, and a combination thereof.

In another preferred embodiment, the amyloid protein regulator is selected from the group consisting of TPPB (CAS. 497259-23-1), rosmarinic acid, and doxycycline.

In another preferred embodiment, the glucocorticoid compound is selected from the group consisting of dexamethasone, hydrocortisone, and a combination thereof.

In another preferred embodiment, the antibiotic is selected from the group consisting of tobramycin, gentamicin sulfate, chlortetracycline, chloramphenicol, and a combination thereof.

In another preferred embodiment, the azole is selected from the group consisting of econazole, isoconazole, bifonazole, clotrimazole, aripiprazole, ketoconazole, fluconazole, phenylimidazole, miconazole. cyproconazole, triadimenol, tebuconazole, propiconazole, and a combination thereof.

In another preferred embodiment, the azole is selected from the group consisting of econazole, fluconazol, tebuconazole, propiconazole, and a combination thereof.

In another preferred embodiment, the mass ratio of the compound of formula I to the azole is 50:1 to 1:50, more preferably 10:1 to 1:10.

In another preferred embodiment, the compound of formula I also comprises a deuterated compound of compound of formula I.

In another preferred embodiment, the pharmaceutical composition or formulation is also used to (b) inhibit, reverse (dissolve or depolymerize) lens protein aggregation.

In another preferred embodiment, the pharmaceutical composition or formulation is also used to (c) prevent and/or treat a disease related to lens protein aggregation.

In another preferred embodiment, the lens protein comprises αB lens protein.

In another preferred embodiment, the disease related to lens protein aggregation is selected from the group consisting of cataract, presbyopia, myopia, cortical opacity, presbyopia nuclear sclerosis, and eye complications caused by diabetes.

In the second aspect of the present invention, a method for non-therapeutically and/or non-diagnostically improving or maintaining lens transparency in vitro is provided, comprising contacting lens with the compound of formula I, or the optical isomer, racemate, solvate, pharmaceutically acceptable salt, prodrug, or deuterated compound thereof to improve (e.g., increase) or maintain lens transparency, wherein the compound of formula I is as defined above.

In the third aspect of the present invention, a method for preventing and/or treating eye diseases caused by lens lesion, comprising administering the compound of formula I, or the optical isomer, racemate, solvate, pharmaceutically acceptable salt, prodrug, or deuterated compound thereof to a subject in need, wherein the compound of formula I is as defined above.

In another preferred embodiment, the subject is human and non-human mammals. Representatively, the non-human mammals comprise (but are not limited to): pet (such as dog and cat), livestock (such as cattle, sheep, horse, and pig), various zoo animals (pandas, elephants) and the like.

In another preferred embodiment, the subject further comprises other animals other than human and non-human mammals, such as non-mammals.

In the fourth aspect of the present invention, a new compound of formula II, or an optical isomer, racemate, solvate, pharmaceutically acceptable salt, prodrug, or a deuterated compound thereof is provided

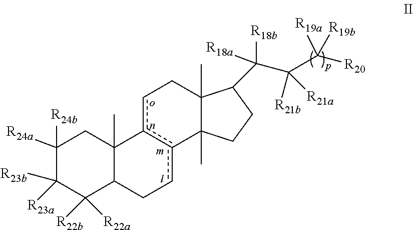

wherein,

p is 0, 1 or 2;

R18a and R18b are each independently hydrogen, substituted or unsubstituted C1-C4 alkyl, or —COOH;

R19a and R19b are each independently hydrogen, substituted or unsubstituted C1-C4 alkyl, or halogen;

R20 is a substituted or unsubstituted C1-C6 acyl, substituted or unsubstituted C1-C6 alkyl, substituted or unsubstituted glycosyl-O—(C1-C6 alkyl)-, substituted or unsubstituted oximido, substituted or unsubstituted C3-C6 heterocycloalkyl, or substituted or unsubstituted C2-C6 alkenyl;

R21a and R21b are each independently hydrogen, hydroxy, thiol, or substituted or unsubstituted C1-C4 alkyl;

R22a and R22b are each independently hydrogen, or substituted or unsubstituted C1-C4 alkyl;

R23a and R23b are each independently hydrogen, hydroxy, thiol, glycosyl, or substituted or unsubstituted C1-C alkoxy;

R24a and R24b are each independently hydrogen, hydroxy, or halogen;

the “substituted” means that one or more hydrogen atoms of the group are substituted by group(s) selected from halogen, —OH, —SH, —COOH, ═O, —CHO, C1-C4 alkyl, C3-C6 cycloalkyl, amino group, glycosyl.

In another preferred embodiment, the glycosyl is a monosaccharide (such as pentose or hexose), or a disaccharide.

In another preferred embodiment, the total number of double bonds represented by the dotted line ( [symbol] ) in the formula II is 0, 1, or 2.

In another preferred embodiment, the double bond is located between m and n.

In another preferred embodiment, the double bond is located between the following positions: m and 1, and/or o and n.

In another preferred embodiment, R22a and/or R22b are methyl.

In another preferred embodiment, p is 1.

In another preferred embodiment, p is 2.

In another preferred embodiment, R23a is hydrogen and R23b is hydroxy, thiol or glycosyl.

In another preferred embodiment, R20 is a glycosyl-O—(C1-C6 alkyl)-.

In another preferred embodiment, the glycosyl is pentosyl or hexosyl, preferably selected from the group consisting of glucosyl, fructosyl, mannosyl, arabinosyl, ribosyl, and a combination thereof.

In another preferred embodiment, the heterocycloalkyl includes 1-3 heteroatoms selected from the group consisting of N, O and S.

In another preferred embodiment, the heterocycloalkyl is a C3-C6 heterocycloalkyl including one 0 hetero atom.

In another preferred embodiment, the compound of formula II has one or more features selected from the group consisting of:

R18a and R18b are each independently —COOH or methyl;

R19a and R19b are each independently hydrogen or halogen;

R20 is hydroxybutyryl, hydroxypropyl, oximido, methoxypropyl, dimethyl epoxyethyl, HOOC—CH═CH—, or hexa-monosaccharide propyl;

R21a and R21b are each independently hydrogen, hydroxy, or thiol;

R22a and R22b are each independently methyl;

R23a and R23b are each independently hydrogen, hydroxy, thiol or hexa-monosaccharide group; and

R24a is hydrogen and R24b is fluorine.

In another preferred example, the same carbon atom does not contain two or more hydroxyl groups.

In another preferred example, the same carbon atom does not contain two or more thiols.

In another preferred embodiment, the hexa-monosaccharide is glucose.

In another preferred embodiment, the compound of formula II is selected from the group consisting of (table B):

TABLE B

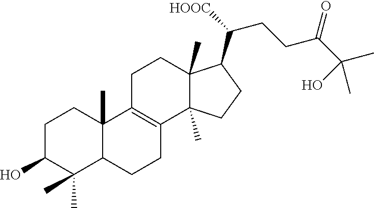

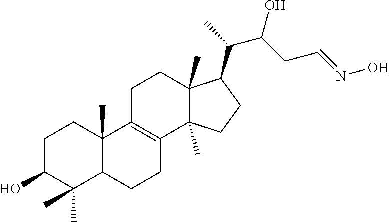

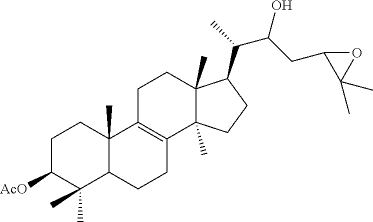

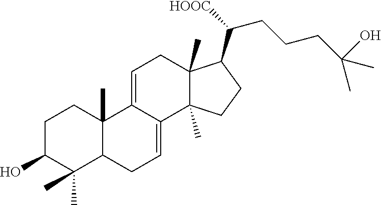

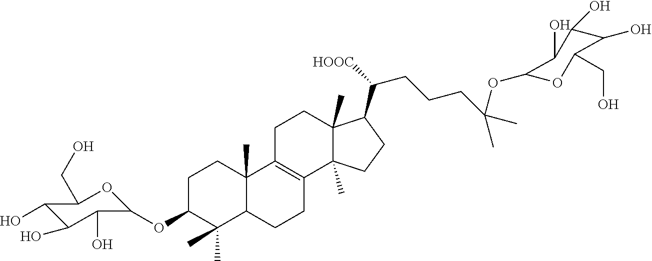

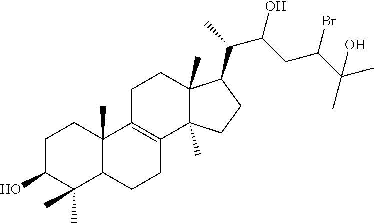

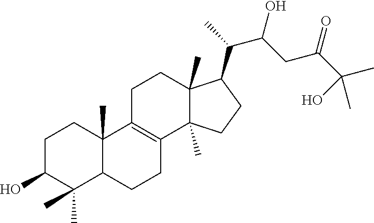

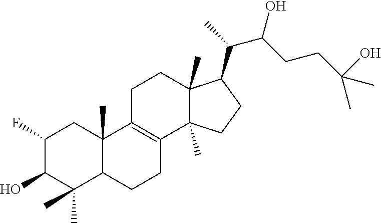

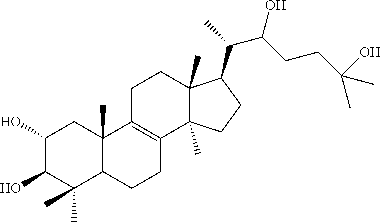

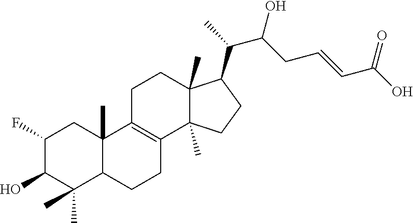

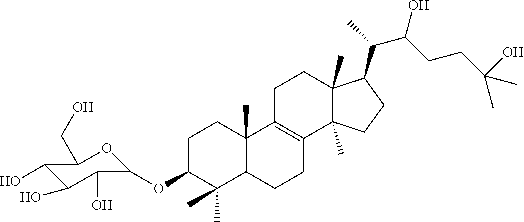

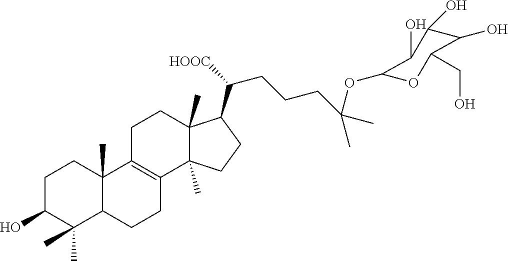

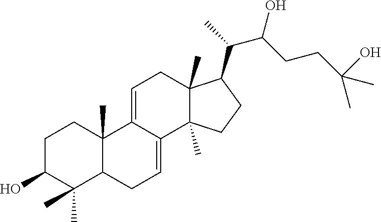

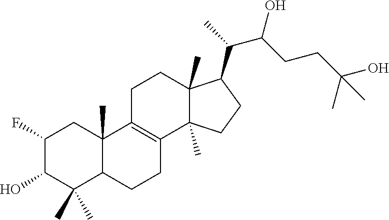

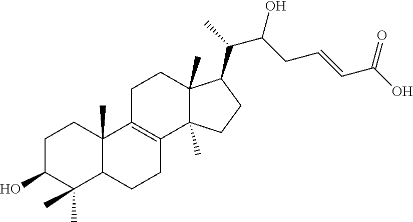

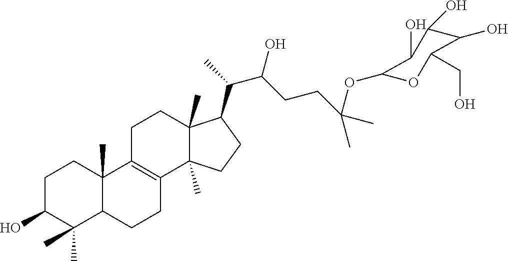

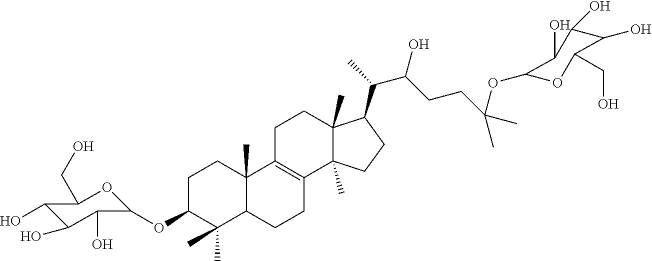

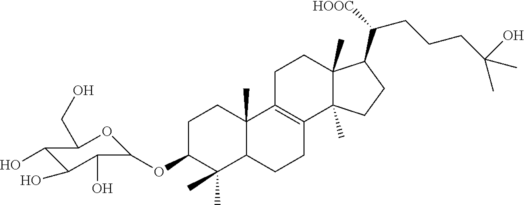

In the fifth aspect of the present invention, a use of a compound of formula II, or the pharmaceutically acceptable salt, or prodrug thereof is provided for the manufacture of a pharmaceutical composition or formulation, the pharmaceutical composition or formulation are used to (a) prevent and/or treat eye diseases caused by lens lesions; (b) inhibit and/or reverse (dissolve or depolymerize) lens protein aggregation; and/or (c) prevent and/or treat a disease related to lens protein aggregation.

In the sixth aspect of the present invention, a use of inonotus obliquus extract is provided for the manufacture of a pharmaceutical composition or formulation, the pharmaceutical composition or formulation are used to (a) prevent and/or treat eye diseases caused by lens lesions.

In another preferred embodiment, the extract comprises liposoluble extract.

In another preferred embodiment, the extract comprises alcohol extract.

In another preferred embodiment, the extract comprises non-aqueous solvent extract.

In another preferred embodiment, the extract is water-insoluble extract or poorly water-soluble extract.

In another preferred embodiment, the extract contains components selected from the group consisting of terpenoids, steroids, and a combination thereof.

In another preferred embodiment, the extract comprises steroids extract.

In another preferred embodiment, the extract comprises triterpenoids, preferably a tetracyclic triterpenoid extract.

In another preferred embodiment, the extract comprises one or more compounds selected from the following table C.

TABLE C

| Com- | |
|---|---|
| pound | |
| no. | Structural formula |
| 1″ | 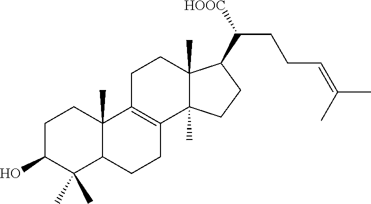 |
| 2″ | 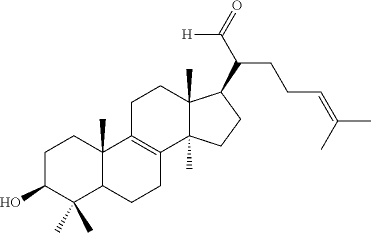 |
| 3″ | 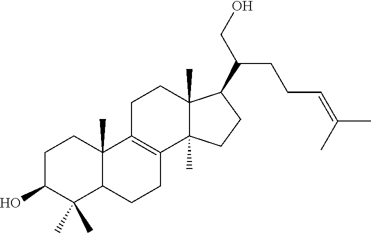 |
| 4″ | 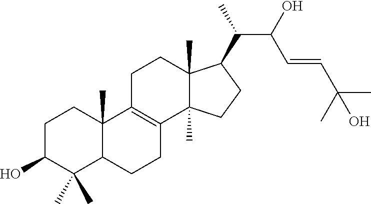 |
| 5″ | 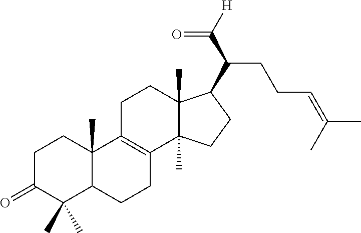 |
| 6″ | 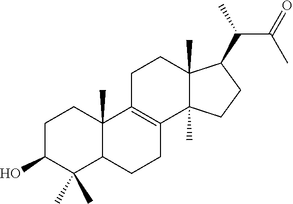 |
| 7″ | 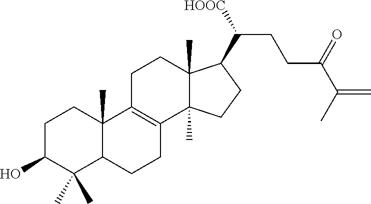 |
| 8″ | 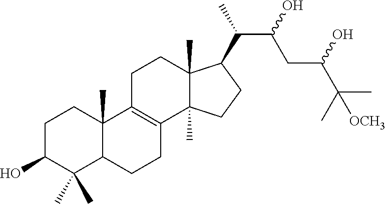 |
| 17″ | 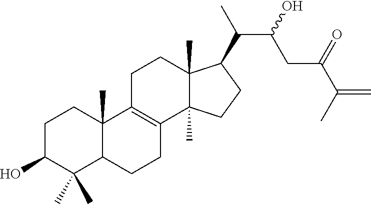 |
| 1 | 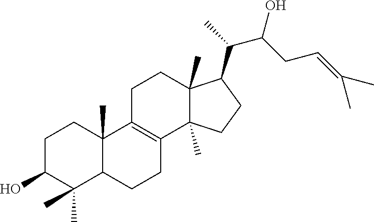 |

Preferably, the extract contains compounds selected from the group consisting of:

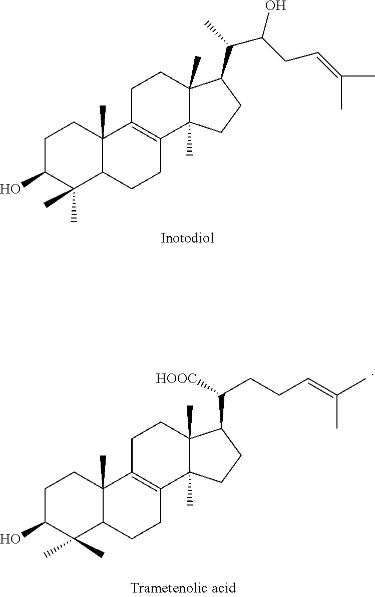

In another preferred embodiment, the extract comprises compounds selected from the group consisting of:

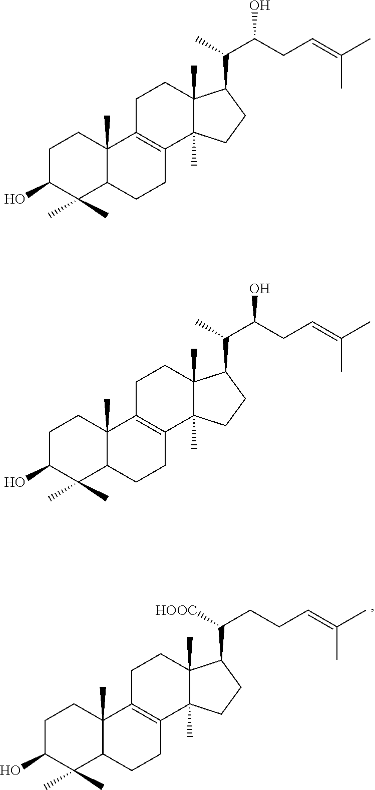

and a combination thereof.

In another preferred embodiment, the weight percentage of the inotodiol is 0.01-99.99 wt %, preferably 1-99 wt % in the extract.

In another preferred embodiment, the weight percentage of the trametenolic acid is 0.01-99.99 wt %, preferably 1-99 wt % in the extract.

In another preferred embodiment, the content of inotodiol and trametenolic acid is 5-100 wt %, preferably 10-100 wt %, more preferably 20-100 wt %, more preferably 30-100 wt %, more preferably 70-100 wt %, most preferably 80-100 wt % in the extract, based on the weight of tetracyclic triterpenoids.

In another preferred embodiment, the extract is not pure lanosterol.

In another preferred embodiment, the weight ratio of inotodiol to lanosterol is ≥2:1, preferably ≥5:1, more preferably ≥10:1 in the extract.

In another preferred embodiment, the extract is C1-C6 alcohol extract of the inonotus obliquus.

In another preferred embodiment, the C1-C6 alcohol comprises methanol, ethanol, propanol, or a combination thereof.

In another preferred embodiment, the extract is further purified after alcohol extraction.

In another preferred embodiment, the further purification comprises extracting with petroleum ether and/or an ester solvent.

In another preferred embodiment, the inonotus obliquus extract comprises liposoluble extract or water-soluble extract of sporophore of inonotus obliquus.

In another preferred embodiment, the eye disease is selected from the group consisting of cataract, presbyopia, myopia, cortical opacity, presbyopia nuclear sclerosis, and eye complications caused by diabetes.

In another preferred embodiment, the eye disease is selected from the group consisting of congenital cataract and acquired cataract.

In another preferred embodiment, the eye disease is selected from the group consisting of mature cataract and immature cataract.

In another preferred embodiment, the cataract is selected from the group consisting of traumatic cataract, metabolic cataract, complicated cataract, senile cataract, spontaneous cataract, or a combination thereof.

In another preferred embodiment, the traumatic cataract comprises surgery-related cataract.

In another preferred embodiment, the spontaneous cataract comprise senile spontaneous cataract.

In another preferred embodiment, the metabolic cataract comprises diabetic metabolic cataract.

In another preferred embodiment, the inonotus obliquus extract is prepared by the following method, comprising steps:

(1). extracting the crude powder of sporophore of inonotus obliquus with alcohol solvent by reflux to obtain an alcohol extract;

(2). suspending the alcohol extract in water, and extracting with petroleum ether to obtain an extract, ie, inonotus obliquus extract; and

optional step (3). subjecting the inonotus obliquus extract to silica gel column chromatography, collecting eluent, and separating to obtain purified inonotus obliquus extract.

In another preferred embodiment, the inonotus obliquus extract is prepared by the following method, comprising steps:

(1). extracting the crude powder of sporophore of inonotus obliquus with ethanol solvent by reflux to obtain an ethanol extract;

(2). suspending the ethanol extract in water, and extracting with petroleum ether, separating solution to obtain the inonotus obliquus extract.

In another preferred embodiment, the method further comprises step (3) after the step (2): subjecting the inonotus obliquus extract to silica gel column chromatography, collecting eluent, and separating to obtain purified inonotus obliquus extract.

In another preferred embodiment, in the step (1), the weight ratio of the crude powder of sporophore of inonotus obliquus to the alcohol solvent is 1:30-100, preferably 1:60-90.

In another preferred embodiment, the alcohol solvent comprises ethanol.

In another preferred embodiment, the alcohol solvent comprises a 90-100% (v/v) ethanol aqueous solution.

In another preferred embodiment, the sporophore is a dry sporophore.

In another preferred embodiment, the pharmaceutical composition or formulation comprises: (a) a therapeutically effective amount of inonotus obliquus extract as the first active ingredient; (b) a pharmaceutically acceptable carrier.

Wherein the content of the first active ingredient is 0.001-99 wt %, preferably 0.01-70% wt %, more preferably 0.05-40% wt %, based on the total weight of the pharmaceutical composition or formulation.

In another preferred embodiment, the dosage form of the pharmaceutical composition or formulation is selected from the group consisting of oral preparation, injection and external preparation.

In another preferred embodiment, the pharmaceutical composition or formulation is an ophthalmic preparation, the ophthalmic preparation is eye drop, emulsion, gel, eye ointment, sustained release microsphere, intraocular sustained-release implant tablet, medicinal sustained-release film.

In another preferred embodiment, the pharmaceutical composition or formulation further comprises: (c) the second active ingredient, wherein the second active ingredient is selected from the group consisting of lanosterols, azoles, glucocorticoid compounds, antibiotics, and a combination thereof.

In another preferred embodiment, the pharmaceutical composition or formulation further comprises: (c) the second active ingredient, wherein the second active ingredient is selected from the group consisting of lanosterols, azoles, glucocorticoid compounds, amyloid protein regulators, antibiotics, or a combination thereof.

In another preferred embodiment, the content of the second active ingredient is 0.01-20 wt %, preferably 5-15 wt %, based on the total weight of the composition.

In another preferred embodiment, the second active ingredient is lanosterols, its concentration is preferably 10-200 mM, more preferably 15-150 mM, more preferably 20-50 mM, most preferably 20-30 mM.

In another preferred embodiment, the lanosterol compound is lanosterol.

In another preferred embodiment, the glucocorticoid compound is selected from the group consisting of dexamethasone, hydrocortisone, and a combination thereof.

In another preferred embodiment, the amyloid protein regulator is selected from the group consisting of TPPB (CAS. 497259-23-1), rosmarinic acid, and doxycycline.

In another preferred embodiment, the antibiotic is selected from the group consisting of tobramycin, gentamicin sulfate, chlortetracycline, chloramphenicol, and a combination thereof.

In another preferred embodiment, the azole is selected from the group consisting of econazole, isoconazole, bifonazole, clotrimazole, aripiprazole, ketoconazole, fluconazole, phenylimidazole, miconazole, cyproconazole, triadimenol, tebuconazole, propiconazole, and a combination thereof.

In another preferred embodiment, the mass ratio of the inonotus obliquus extract to the azole is 50:1 to 1:50, preferably 10:1 to 1:10.

In another preferred embodiment, the pharmaceutical composition or formulation is also used to (b) inhibit, reverse lens protein aggregation; and/or (c) prevent and/or treat a disease related to lens protein aggregation.

In another preferred embodiment, the lens protein comprises αB lens protein.

In another preferred embodiment, the disease related to lens protein aggregation is selected from the group consisting of cataract, presbyopia, myopia, cortical opacity, presbyopia nuclear sclerosis, and eye complications caused by diabetes.

In the seventh aspect of the present invention, a method for non-therapeutically and/or non-diagnostically improving or maintaining lens transparency in vitro is provided, comprising contacting lens with the inonotus obliquus extract to improve (or increase) or maintain lens transparency, wherein the inonotus obliquus extract is as described in the fourth aspect of the present invention.

In the eighth aspect of the present invention, a method for preventing and/or treating eye diseases caused by lens lesion, comprising administering the inonotus obliquus extract to a subject in need thereof, wherein the inonotus obliquus extract is as described in the sixth aspect of the present invention.

In another preferred embodiment, the subject is human and non-human mammals. Typically, the non-human mammals comprise (but are not limited to) pet (such as dog and cat), livestock (such as cattle, sheep, horse, pig), various zoo animals (pandas, elephant) and the like.

In another preferred embodiment, the subject further comprises other animals other than human and non-human mammals, such as non-mammals.

In the ninth aspect of the present invention, a pharmaceutical composition is provided, which comprises:

(a) a therapeutically effective amount of the compound of formula I or formula II, or the optical isomer, racemate, solvate, pharmaceutically acceptable salt, prodrug, or deuterated compound thereof as the first active ingredient;

(b) a pharmaceutically acceptable carrier; and

(c) the second active ingredient, which is selected from the group consisting of an azole compound, an amyloid modulator, or a combination thereof.

Wherein the content of the first active ingredient is 0.001-99 wt %, preferably 0.01-70% wt %, more preferably 0.05-40% wt %, based on the total weight of the composition.

In another preferred embodiment, the concentration of the first active ingredient is preferably 1-500 mM or 10-200 mM, preferably 15-150 mM, more preferably 20-50 mM, most preferably 20-30 mM.

In another preferred embodiment, the content of the second active ingredient is 0.01-20 wt %, preferably 5-15 wt %, based on the total weight of the composition.

In another preferred embodiment, the second active ingredient is azoles, its concentration is preferably 10-200 mM, more preferably 15-150 mM, more preferably 20-50 mM, most preferably 20-30 mM.

In another preferred embodiment, the azole compound is selected from the group consisting of econazole, isoconazole, bifonazole, clotrimazole, aripiprazole, ketoconazole, fluconazole, phenylimidazole, miconazole, cyproconazole, triadimenol, tebuconazole, propiconazole, and a combination thereof.

In another preferred embodiment, the azole compound is selected from the group consisting of econazole, fluconazol, isoconazole, tebuconazole, propiconazole, and a combination thereof.

In another preferred embodiment, the amyloid protein regulator is selected from the group consisting of TPPB (CAS. 497259-23-1), rosmarinic acid, and doxycycline.

In another preferred embodiment, the mass ratio of the compound of formula I (or the compound of formula II) to the azole compound is 50:1 to 1:50, preferably 10:1 to 1:10.

In another preferred embodiment, the compound of formula I is selected from the group consisting of inotodiol, trametenolic acid, and a combination thereof.

In another preferred embodiment, the dosage form of the pharmaceutical composition or formulation is selected from the group consisting of injection, external preparation and oral preparation.

In another preferred embodiment, the pharmaceutical composition or formulation is an ophthalmic preparation, the ophthalmic preparation is eye drop, emulsion, gel, eye ointment, sustained release microsphere, intraocular sustained-release implant tablet, medicinal sustained-release film.

It should be understood that, in the present invention, each of the technical features specifically described above and below (such as those in the Examples) can be combined with each other, thereby constituting new or preferred technical solutions which need not be specified again herein due to space limitation.

### DESCRIPTION OF THE DRAWINGS

FIG. 1 shows the morphology of the upper light source of the rat lens after successful cataract modeling in example 1 group of the present invention.

FIG. 2 shows the morphology of the lower light source of the rat lens after successful cataract modeling in example 1 group of the present invention.

FIG. 3 shows the morphology of the upper light source of the rat lens after successful cataract modeling in Comparative example 1 group of the present invention.

FIG. 4 shows the morphology of the lower light source of the rat lens after successful cataract modeling in Comparative example 1 group of the present invention.

FIG. 5 shows the morphology of the upper light source of the rat lens after the end of the experiment in example 1 group of the present invention.

FIG. 6 shows the morphology of the lower light source of the rat lens after the end of the experiment in example 1 group of the present invention.

FIG. 7 shows the morphology of the upper light source of the rat lens after the end of the experiment in Comparative example 1 group of the present invention.

FIG. 8 shows the morphology of the lower light source of the rat lens after the end of the experiment in Comparative example 1 group of the present invention.

FIG. 9 shows the morphology of the upper light source of the rat lens after successful cataract modeling in example 2 group of the present invention.

FIG. 10 shows the morphology of the lower light source of the rat lens after successful cataract modeling in example 2 group of the present invention.

FIG. 11 shows the morphology of the upper light source of the rat lens after successful cataract modeling in Comparative example 2 group of the present invention.

FIG. 12 shows the morphology of the lower light source of the rat lens after successful cataract modeling in Comparative example 2 group of the present invention.

FIG. 13 shows the morphology of the upper light source of the rat lens after the end of the experiment in example 2 group of the present invention.

FIG. 14 shows the morphology of the lower light source of the rat lens after the end of the experiment in example 2 group of the present invention.

FIG. 15 shows the morphology of the upper light source of the rat lens after the end of the experiment in Comparative example 2 group of the present invention.

FIG. 16 shows the morphology of the lower light source of the rat lens after the end of the experiment in Comparative example 2 group of the present invention.

FIG. 17 shows the morphology of an eyeball before and after the treatment of congenital cataract (left eye) of cynomolgus monkey with inotodiol eye ointment in Example 8 of the present invention, wherein 17A is before administration and 17B is after administration.

FIG. 18 shows the morphology of an eyeball before and after the treatment of congenital cataract (right eye) of cynomolgus monkey with inotodiol eye ointment in Example 8 of the present invention, wherein 18A is before administration and 18B is after administration.

FIG. 19 shows the morphology of an eyeball before and after the treatment of spontaneous cataract (left eye) of cynomolgus monkey (No. 013321) with inotodiol eye ointment in Example 9 of the present invention, wherein 19A is before administration and 19B is after administration.

FIG. 20 shows the morphology of an eyeball before and after the treatment of spontaneous cataract (left eye) of cynomolgus monkey (No. 990447) with inotodiol eye ointment in Example 9 of the present invention, wherein 20A is before administration and 20B is after administration.

FIG. 21 shows the morphology of an eyeball before and after the treatment of spontaneous cataract (left eye) of cynomolgus monkey (No. 993747) with lanosterol eye ointment in Example 9 of the present invention, wherein 21A is before administration and 21 B is after administration.

FIG. 22 shows the morphology of an eyeball before and after the treatment of traumatic cataract of rat (the right eye of a female rat is selected as comparison) with inotodiol eye ointment in Example 10 of the present invention, wherein 22A is before administration and 22 B is after administration.

FIG. 23 shows the morphology of an eyeball before and after the treatment of traumatic cataract of rat (the right eye of a male rat is selected as comparison) with trametenolic acid eye ointment in Example 10 of the present invention, wherein 23A is before administration and 23 B is after administration.

FIG. 24 shows the morphology of an eyeball before and after the treatment of traumatic cataract of rat (the left eye of a female rat is selected as comparison) with lanosterol eye ointment in Example 10 of the present invention, wherein 24 A is before administration and 24 B is after administration.

FIG. 25 shows the morphology of an eyeball before and after the treatment of metabolic cataract of diabetic rat (the right eye of a male rat is selected as comparison) with inotodiol eye ointment in Example 11 of the present invention, wherein 25A is before administration and 25 B is after administration.

FIG. 26 shows the morphology of an eyeball before and after the treatment of metabolic cataract of diabetic rat (the right eye of a male rat is selected as comparison) with trametenolic acid eye ointment in Example 11 of the present invention, wherein 26A is before administration and 26 B is after administration.

FIG. 27 shows the morphology of an eyeball before and after the treatment of metabolic cataract of diabetic rat (the right eye of a male rat is selected as comparison) with lanosterol eye ointment in Example 11 of the present invention, wherein 27A is before administration and 27B is after administration.

FIG. 28 shows the morphology of an eyeball before and after the treatment of metabolic cataract of diabetic rat (the right eye of a male rat is selected as comparison) with blank eye ointment in Example 11 of the present invention, wherein 28A is before administration and 28B is after administration.

FIG. 29 shows the morphology of an eyeball before and after the right eye treatment of metabolic cataract of diabetic rat with pirenoxine sodium eye drops (as positive control group) in Example 11 of the present invention, wherein 29 A is before administration and 29 B is after administration.

FIG. 30 shows a nuclear magnetic resonance (NMR) of the inonotus obliquus extract 2 prepared in Example 14 of the present invention.

FIG. 31 shows a mass spectrum (MS) of the inonotus obliquus extract 2 prepared in Example 14 of the present invention.

FIG. 32 shows a high performance liquid chromatography (HPLC) of the inonotus obliquus extract 2 prepared in Example 14 of the present invention.

FIG. 33 shows the detection result of ThT fluorescence signal of lens protein aggregate is significantly reduced by inotodiol in Example 22 of the present invention.

### EMBODIMENTS FOR CARRYING OUT THE INVENTION

Based on a long-term and intensive research, the inventors have unexpectedly discovered at the first time that the compound of formula I (including pure substance, mixtures or corresponding extracts) can significantly prevent and/or treat eye diseases caused by lens lesions. The experimental results of the present invention showed that the compound of formula I can quickly cure and prevent eye diseases caused by lens lesions in a simple administration mean. The present invention is completed on this basis.

### Terms

As used herein, the term “R1”, “R₁” and “R1” have the same meaning, and other similar definitions have the same meaning.

As used herein, the term “C1-C20 alkyl” or “C1-C10 alkyl” or “C1-C8 alkyl” refers to a linear or branched alkyl with 1-20 or 1-10 or 1-8 carbon atoms, such as methyl, ethyl, propyl, isopropyl, butyl, isobutyl, sec-butyl, tert-butyl, pentyl, hexyl, heptyl, octyl, or the like.

As used herein, the term “C1-C8 alkoxy” or “C1-C4 alkoxy”” refers to a straight or branched alkoxy having 1 to 8 or 1 to 4 carbon atoms, such as methoxy, ethoxy, propoxy, isopropoxy, butoxy, isobutoxy, sec-butoxy, tert-butoxy, pentyloxy, hexyloxy, heptyloxy, octyloxy, or the like.

As used herein, the term “C1-C8 alkylthio” or “C1-C4 alkylthio” refers to a straight or branched alkylthio having 1 to 8 or 1 to 4 carbon atoms, such as methyl thio, ethyl thio, propyl thio, isopropyl thio, butyl thio, isobutyl thio, sec-butyl thio, tert-butyl thio, pentyl thio, hexyl thio, heptyl thio, octyl thio, or the like.

As used herein, the term “C3-C10 cycloalkyl” refers to a cycloalkyl having from 3 to 10 carbon atoms, such as cyclopropyl, cyclobutyl, methylcyclobutyl, cyclopentyl, or the like.

As used herein, the term “C1-C8 alkylene” or “C1-C7 alkylene” or “C1-C6 alkylene” refers to a group having “—CH2-” formed by one carbon atom of linear or branched alkyl with 1-8 or 1-7 or 1-6 carbon atoms losing two hydrogen atoms, eg, methylene (—CH2-) or the like.

As used herein, the term “C2-C10 alkenyl” refers to a hydrocarbon radical formed by a straight or branched alkene with 2 to 10 carbon atoms having one or more double bonds losing one hydrogen atom bonded to the double bond, such as vinyl (CH2=CH—), (C(CH3)₂═CH—), or the like.

As used herein, the term “C2-C8 alkynyl” refers to a hydrocarbon radical formed by a straight or branched alkyne with 2 to 8 carbon atoms having one or more triple bonds losing one hydrogen atom bonded to the triple bond such as, ethynyl (CH═CH—), (H3C—C≡CH—), or the like.

As used herein, the term “C1-C6 haloalkyl” refers to a substituent having a straight or branched alkyl-halogen structure with 1 to 6 carbon atoms, such as —CH₂Cl, —CH₂CH₂Br, CH₂CHCH₂Cl, or the like.

As used herein, the term “halogen” refers to F, Cl, Br, and I.

As used herein, the term “aryl” refers to a C6-C20 aryl including monocyclic or bicyclic or tricyclic aryl, such as phenyl, naphthyl, anthryl.

As used herein, the term “C6-C10 member aryl” refers to a cyclic group having an aromatic structure, such as phenyl, naphthyl.

As used herein, the term “5-8 membered heteroaryl” refers to a heteroaryl having 5-8-membered monocyclic or fused polycyclic with N, O or S on the ring system, such as pyrrolyl, pyridyl, furyl, or the like.

As used herein, the term “cyanate group” has the formula: —O—C≡N.

As used herein, the term “isocyanate group” has the formula: —N═C═O.

As used herein, the term “isothiocyanate group” has the formula: —N═C═S.

As used herein, the term “sulfonamido” has the formula:

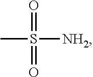

or the like.

As used herein, the term “oximido” has the formula:

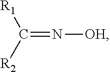

wherein, R₁ and R₂ represent the linked groups respectively, and R₁ and R₂ can be the same or different.

As used herein, the term “glycosyl” refers to a monovalent substituent formed by the removal of a hemiacetal hydroxyl group from a cyclic form of a monosaccharide (or disaccharide). Representative monosaccharide comprises pentose and hexose. Preferred glycosyl is O-glycosyl formed by monosaccharide substituting one or more OH groups of the compound of formula I. As used herein, the terms “comprising”, ““comprise”, “containing” are used interchangeably, which not only include closed definition but also semi-closed, and open definition. In other words, the terms comprise “consist of”, “consist essentially of”.

As used herein, the term “deuterated (D)” means that one or more hydrogens in a compound or group are replaced by deuterium. Deuterated can be monosubstituted, disubstituted, polysubstituted or fully substituted. In another preferred embodiment, the deuterium isotope content at the deuterium substitution site is greater than the natural deuterium isotope content (0.015%), preferably greater than 50%, more preferably greater than 75%, and more preferably greater than 95%, more preferably greater than 97%, more preferably greater than 99%, and more preferably greater than 99.5%. In another preferred embodiment, D has an isotope content of ≥95%, more preferably ≥99% at the position of the hydrogen atom in the compound.

As used herein, the term “presbyopia”, also known as “presbytism”, refers to the visual state in which the eye lens loses its flexibility, making it difficult to focus on close objects. Typically, the eye diseases caused by the lens lesions do not include conjunctivitis, eye inflammation or eye infections caused by pathogens (such as bacteria, viruses, etc.).

As used herein, the term “prevention” refers to the administration of a therapeutically effective amount of the compound of formula I (including pure substance, mixtures), or the optical isomer, racemate, solvate, pharmaceutically acceptable salt, prodrug, or deuterated compound thereof, and/or inonotus obliquus extract prior to eye disease, thereby making eye disease occurrence be prevented and delayed, or the diseases still occur, but the degree of eye diseases is reduced compared with no application of compound of formula I, or the optical isomer, racemate, solvate, pharmaceutically acceptable salt, prodrug thereof, and/or inonotus obliquus extract. As used herein, the term “treatment” refers to administrate a therapeutically effective amount of the compound of formula I, or the optical isomer, racemate, solvate, pharmaceutically acceptable salt, or prodrug thereof, thereby reducing, alleviating or slowing down the progress or development of eye diseases, disorders or symptoms. In another embodiment, the “treatment” refers to alleviate or slow down the progress of eye diseases, disorders or symptom, or improve one or more signs or symptoms of eye diseases, disorders or symptoms.

As used herein, the term “preventment” and “treatment” comprise delaying and terminating the progress of the disease, or eliminating eye diseases caused by lens lesions without requiring 100% inhibition, elimination and reversal. In some embodiment, the compound of formula I and/or inonotus obliquus extract, and compositions and formulation thereof in the present invention reduce, prevent, inhibit and/or reverse eye diseases (such as cataracts) caused by lens lesions such as at least 1%, at least 10%, at least 30%, at least 50%, or at least 80%, compare with the level observed by no application of the compound of formula I and/or inonotus obliquus extract, and compositions and formulation thereof in the present invention (e.g., in biologically matched control subjects or specimens that are not exposed to the compound of formula I and/or inonotus obliquus extract, and compositions and formulation thereof in the present invention).

In another preferred example, the term “cataract” refers to a disease or condition that causes opacity (turbid) or unclarity on the surface and/or inside the lens, or induces lens swelling, comprising congenital cataracts and acquired cataracts. Typically, the cataract comprises (but is not limited to): age-related cataract, diabetic cataract, surgery-related cataract, radiation-induced cataract, cataract caused by genetic diseases, cataract caused by infection, or drug-induced cataract.

### Active Ingredient

The present invention discovers for the first time the use of the compound of the formula I for treating eye diseases related to lens lesions. The compound of the formula I is used to prepare a pharmaceutical composition or preparation, and the pharmaceutical composition or preparation is used to prevent and/or treat eye diseases caused by lens lesions.

As used herein, the term “compound of the present invention”, “compound of formula I” are used interchangeably, which refer to a compound of formula I, or an optical isomer, racemate, solvate, pharmaceutically acceptable salt, or prodrug thereof. In addition, the term also comprises the corresponding deuterated compounds. It should be understood that the term also comprises a mixture of the above components. In the compound of formula I, if there is a chiral carbon atom, the chiral carbon atom can be R configuration, S configuration, or a mixture of the both.

The structure of the compound of formula I is as described above in the first aspect of the invention.

In another preferred embodiment, in the structural formula, the total number of double bonds represented by the dotted line ( [symbol] ) in the structural formula is 0, 1, 2, 3 or 4.

In another preferred embodiment, the double bond is located between the following positions: a and b, b and c, c and d, e and f, c and f, f and g, g and h, h and a, b and i, i and j, a and k, and/or k and l.

In another preferred embodiment, the valence state of each C conforms to the requirements of the chemically stable structure (ie, C is tetravalent) in the structural formula.

In another preferred embodiment, R1a, R1b, R2a, R2b, R3a and R3b are each independently selected from hydrogen, —OH, —CHO, —COOH, —SH, substituted or unsubstituted C1-C3 alkyl-OCO—, substituted or unsubstituted C1-C4 alkyl, -A-B, or R1a and Rb, R2a and R2b, and/or R3a and R3b constitute ═O; wherein, A is independently a substituted or unsubstituted C1-C4 alkylene, and B is independently —OH, —SH, C1-C3 alkoxy, —CHO, —COOH, ═O.

In another preferred embodiment, Y is a group having a hydroxyl group or SH substituent.

In another preferred embodiment, R4 is hydrogen or methyl.

In another preferred embodiment, R7, R12 and R15 are each independently selected from absent, hydrogen, methyl.

In another preferred embodiment, R13a and R13b are each independently selected from hydrogen, methyl, —OH, —SH, ═O, halogen, and at most one of R13a and R13b is absent.

In another preferred embodiment, R10a and R10b are each independently selected from hydrogen, —OH, —SH, —OSO₃H, —OPO₃H, —COOH, —CHO; or R10a and R10b constitute a carbonyl.

In another preferred embodiment, R5, R6, R8, R9a, R9b, R14, R16, R17a and R17b are each independently selected from hydrogen and methyl.

In another preferred embodiment, Z is not —CH₂C(CH₃)₃.

In another preferred embodiment, Z is —CH₂C(CH₃)₂—OH.

In another preferred example, the compound of formula I is any of the compounds of formula I-1, I-2, I-3, I-4, I-5, I-6, I-7 and I-8.

In another preferred embodiment, the compound of formula I is selected from the group consisting of:

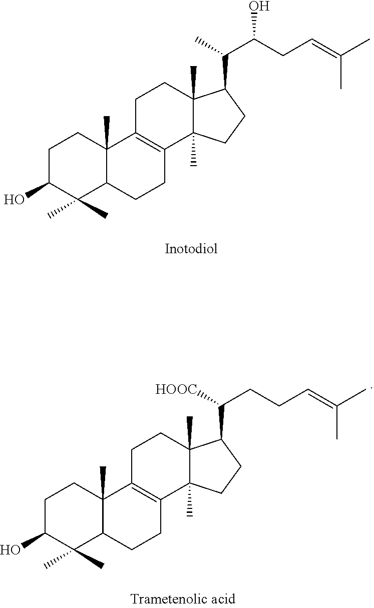

In the present invention, the term “pharmaceutically acceptable salt” refers to a salt suitable for use as a medicament formed by the compound of the present invention with an acid or base. Pharmaceutically acceptable salts include inorganic and organic salts. A preferred salt is a salt formed by the compounds of the present invention and acid. Suitable salt-forming acids include (but are not limited to): inorganic acids such as hydrochloric acid, hydrobromic acid, hydrofluoric acid, sulfuric acid, nitric acid, phosphoric acid; organic acids such as formic acid, acetic acid, propionic acid, oxalic acid, malonic acid, succinic acid, fumaric acid, maleic acid, lactic acid, malic acid, tartaric acid, citric acid, picric acid, methanesulfonic acid, benzoic acid, and benzenesulfonic acid; and acidic amino acids such as aspartic acid, glutamic acid. A preferred salt is a salt formed by the compounds of the present invention and base. Suitable salt-forming bases include (but are not limited to): inorganic bases such as sodium hydroxide, potassium hydroxide, sodium carbonate, sodium hydrogencarbonate, sodium phosphate; and organic bases such as ammonia, triethylamine, diethylamine.

In the present invention, the term “prodrug”, also referred to pro-drug, drug precursor, precursor, etc., refers to a compound which has a pharmacological action after being transformed in vivo. The prodrug itself has no biological activity or low activity, and becomes an active substance after metabolism in the body. The process aims to increase the bioavailability of the drug, enhance the target ability, and reduce the toxicity and side effects of the drug. In the present invention, the prodrug of compound of formula A can be metabolized to the compound of formula I (representatively, such as inotodiol). In the present invention, a preferred prodrug is an ester formed by esterification of the compound of formula I of the present invention with a compound containing hydroxyl or a compound containing carboxyl. Representatively, the compound containing hydroxyl comprises C1-C6 lower alcohol (e.g., ethanol, propanol, etc.), low grade sugar (e.g., glucose, lactose, etc.). Representatively, the compound containing carboxyl comprises C1-C6 lower acid. Typically, the C1-C6 lower acid comprises organic or inorganic acid. For example, the C1-C6 lower acid is hydrochloric acid, sulfuric acid, acetic acid, propionic acid, oxalic acid, fumaric acid, maleic acid, malic acid, tartaric acid, etc. Another preferred prodrug is glycosylated compound, i.e., one or more OH groups in the compound of formula I (e.g., R10a, R10b, Z or other positions in formula 1) are substituted with a glycosyl to form O-glycosylation product.

The compound of formula I of the present invention can be prepared by a method well known to the skilled in the art, and the reaction parameters of the respective steps are not particularly limited. Furthermore, the compound of the invention is also commercially available. Typically, the compound of formula I of the present invention is obtained from the extraction, separation and purification of inonotus obliquus.

In the present invention, the inventors conducted pharmacokinetic studies on different compounds of formula I, the results showed that the half-life of the compound of formula I was significantly prolonged in vivo when at least one of Ra, R2a, R3a, R1b, R2b and R3b is a group containing O or S (e.g. —OH or —SH) in the structure of the compound of formula I, particularly, the concentration of aggregation in the eye was increased, the residence time in aqueous humor was prolonged, and the therapeutic effect on eye diseases was improved.

Use of the Compound of the Present Invention

The compound of formula I, or an optical isomer, racemate, solvate, pharmaceutically acceptable salt, prodrug, or a deuterated compound thereof is provided for the preparation of a pharmaceutical composition or formulation, the pharmaceutical composition or formulation are used to (a) prevent and/or treat eye diseases caused by lens lesions.

In another preferred embodiment, the pharmaceutical composition or formulation is also used to (b) inhibit, reverse (dissolve or depolymerize) lens protein aggregation; and/or (c) prevent and/or treat a diseases related to lens protein aggregation.

In another preferred example, the eye diseases comprise (but are not limited to): cataract, presbyopia, myopia, cortical opacity, presbyopia nuclear sclerosis, eye complications caused by diabetes.

Inonotus Obliquus Extract and Preparation Method Thereof

The present invention has firstly discovered the use of the inonotus obliquus extract in the treatment of eye diseases related to lens lesions, the inonotus obliquus extract is use for the preparation of a pharmaceutical composition or formulation, the pharmaceutical composition or formulation are used to prevent and/or treat eye diseases caused by lens lesions.

As used herein, the term “extract” or “effective part” comprises water-soluble and/or liposoluble extract. The term also comprises alcohol extract or aqueous extract, especially non-aqueous, or poorly water-soluble extract or liposoluble extract. Further, the term also comprises an effective part group, i.e. an extract containing liposoluble effective part and water-soluble effective part, or a mixture thereof.

It is analyzed that the chemical component contained in the effective part in the present invention comprises at least the substances selected from the group consisting of terpenoids, steroids, or a combination thereof.

The method for preparing the inonotus obliquus extract of the present invention is not particularly limited. A water-soluble and/or liposoluble extract can be obtained by a conventional method using inonotus obliquus as a raw material.

In a preferred embodiment of the invention, the preparation of the active part is carried out by solvent extraction method, extraction method, and/or chromatography.

In the present invention, the solvent used in the solvent extraction method is not particularly limited, and representative examples comprise (but are not limited): one of water, ethanol, methanol, acetone, ethyl acetate or a mixed solvent of several solvents. The number of extractions can be one or more times.

In the present invention, the solvent used in the solvent extraction method is not particularly limited, and representative examples comprise (but are not limited): one of n-butanol, ethyl acetate, dichloromethane, chloroform, cyclohexane, petroleum ether or a mixed solvent of several solvents. The number of extractions can be one or more times.

In the present invention, column chromatography in the chromatography is not particularly limited, and representative examples comprise (but are not limited): one of activated carbon, silica gel, reversed phase silica gel, macroporous resin, and dextran gel or a combination thereof.

In a preferred embodiment, the extract comprises terpenoid component, such as triterpenoid component. Preferably, the extract comprises tetracyclic triterpenoid component.

In a preferred embodiment, the extract comprises a mixture of recombination of two or more extracts obtained from by inonotus obliquus using different extraction methods.

In a preferred embodiment, the extract contains a compound selected from the group consisting of:

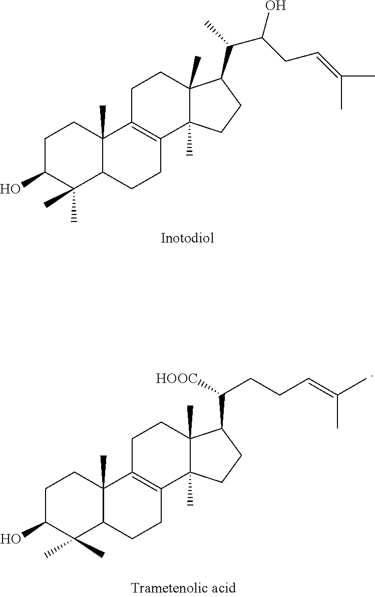

In another preferred embodiment, the weight percentage of the inotodiol is 0.01-99.99 wt %, in the extract. In another preferred embodiment, the weight percentage of the trametenolic acid is 0.01-99.99 wt % in the extract.

In one embodiment of the invention, a method for preparing the inonotus obliquus extract is provided, comprising steps:

(1). extracting the crude powder of dried sporophore of inonotus obliquus with ethanol by reflux to obtain an ethanol extract;

(2). suspending the ethanol extract in water, and extracting with petroleum ether, separating solution to obtain the inonotus obliquus extract.

In another preferred embodiment, the method further comprises step (3) after step (2): comprising subjecting the inonotus obliquus extract to silica gel column chromatography, collecting eluent, and separating to obtain the purified inonotus obliquus extract.

In another preferred embodiment, a method for preparing inotodiol from inonotus obliquus is provided, comprising steps:

First, the crude powder of dried sporophore of inonotus obliquus is extracted with ethanol by reflux, the solvent in extract solution is removed by reduced pressure distillation, and the ethanol extract is obtained. The ethanol extract is suspended with water, and extracted with petroleum ether and ethyl acetate successively, then the solvent in extract solution is removed by reduced pressure distillation, petroleum ether extract and ethyl acetate extract are obtained. The petroleum ether extract is subjected to silica gel column chromatography and eluted gradiently with petroleum ether-ethyl acetate (e.g., 15:1-2:1). Each time the eluate is collected, according to the results of thin layer chromatography and the same fractions are combined. After removing the solvent by reduced pressure distillation, the single component is recrystallized. Inotodiol is obtained by eluting with petroleum ether-ethyl acetate (e.g. about 5:1); or the ethyl acetate extract is subjected to silica gel column chromatography, elution with gradient chloroform-methanol (e.g. 100:1-10:1), then elution with chloroform-methanol (e.g. 70:1) to obtain mixture I, then the mixture I is eluted with gradient petroleum ether-ethyl acetate (e.g. 30:1-1:1), then eluted with petroleum ether-ethyl acetate (e.g. 8:1) to obtain inotodiol.

In another preferred embodiment, a method for extracting trametenolic acid from inonotus obliquus is provided, comprising steps:

First, the crude powder of dried sporophore of inonotus obliquus is extracted with ethanol by reflux, the solvent in extract solution is removed by reduced pressure distillation, and the ethanol extract is obtained. The ethanol extract is suspended with water, and extracted with petroleum ether and ethyl acetate successively, then the solvent in extract solution is removed by reduced pressure distillation, petroleum ether extract and ethyl acetate extract are obtained. The ethyl acetate extract is subjected to silica gel column chromatography, eluting gradiently with chloroform-methanol (e.g., 100:1-10:1), then eluting with chloroform-methanol (e.g., 50:1) to obtain mixture II. The mixture II is subjected to silica gel column, eluting gradiently with petroleum ether-ethyl acetate (e.g., 20:1-1:2), then eluting with petroleum ether-ethyl acetate (e.g., 3:1) to obtain mixture III. The mixture III is subjected to silica gel column, eluting gradiently with petroleum ether-ethyl acetate (e.g., 5:1 to 1:2), then eluting with petroleum ether-ethyl acetate (e.g., 2:1) to obtain trametenolic acid.

Use of the Inonotus Obliquus Extract

The present invention has firstly provided the inonotus obliquus extract for preparation of a pharmaceutical composition or formulation, the pharmaceutical composition or formulation is used to prevent and/or treat eye diseases caused by lens lesions.

In another preferred embodiment, the pharmaceutical composition or formulation is also used to (b) inhibit, reverse lens protein aggregation; and/or (c) prevent and/or treat a disease related to lens protein aggregation.

In another preferred example, the eye diseases comprise (but are not limited to): cataract, presbyopia, myopia, cortical opacity, presbyopia nuclear sclerosis, eye complications caused by diabetes.

Pharmaceutical Composition or Formulation and Administration Method

Because the compound and the inonotus obliquus extract of the present invention can significantly prevent and/or treat the eye diseases caused by lens lesions, the pharmaceutical composition or formulation containing the compound of the present invention as the active ingredient and/or the inonotus obliquus extract of the present invention can be used to (a) prevent and/or treat eye diseases caused by lens lesions; (b) inhibit, reverse (dissolve or depolymerize) lens protein aggregation; and/or (c) prevent and/or treat a diseases related to lens protein aggregation. The pharmaceutical composition or formulation comprises: the compound of formula I and/or an inonotus obliquus extract of the present invention; and a pharmaceutically acceptable carrier.

Wherein the content of the first active ingredient is 0.001-99 wt %, preferably 0.01-70% wt %, more preferably 0.05-40% wt %, based on the total weight of the composition.

In another preferred embodiment, the pharmaceutical composition or formulation further comprises: (c) the second active ingredient, wherein the second active ingredient is selected from the group consisting of lanolin compounds, lanosterols, any compound contained in fungi in all of Hymenochaetales or Polyporales, azoles, myloid protein regulator, glucocorticoid compounds, antibiotics, and a combination thereof.

In another preferred embodiment, the content of the second active ingredient is 0.01-20 wt %, preferably 5-15 wt %, based on the total weight of the composition.

In another preferred embodiment, the second active ingredient is lanolin compound, its concentration is preferably 10-200 mM, more preferably 15-150 mM, more preferably 20-50 mM, most preferably 20-30 mM.

In another preferred embodiment, the lanolin compound is lanosterol.

In another preferred embodiment, the glucocorticoid compound is selected from the group consisting of dexamethasone, hydrocortisone, and a combination thereof.

In another preferred embodiment, the antibiotic is selected from the group consisting of tobramycin, gentamicin sulfate, chlortetracycline, chloramphenicol, and a combination thereof.

In another preferred embodiment, the azole is selected from the group consisting of econazole, isoconazole, bifonazole, clotrimazole, aripiprazole, ketoconazole, fluconazole, phenylimidazole, miconazole. cyproconazole, triadimenol, tebuconazole, propiconazole, and a combination thereof.

As used herein, the term “myloid protein regulator”, also referred to amyloid modulator, amyloid inhibitor, amyloid-like inhibitor, specifically refers to a kind of molecule or compound that can regulate or inhibit protein accumulation, adhesion and precipitation, and affect the formation of fibrillary protein aggregates.

As used herein, the term “therapeutically effective amount” refers to an amount that produces function or activity to a human and/or animal and is acceptable to humans and/or animals. It will be understood by the skilled in the art that the “effective amount” or “effective dose” can vary with the form of the pharmaceutical composition, the route of administration, the pharmaceutical excipients, the disease severity, and the combination of other drugs, and the like.

The term “pharmaceutically acceptable carrier” refer to one or more compatible solid, semi-solid, liquid or gel fillers, which are suitable for use in humans or animals and must have sufficient purity and sufficient low toxicity. The “compatible” refers to each ingredient of the pharmaceutical composition and the active ingredient of the drug can be blended with each other without significantly reducing the efficacy.

It should be understood that the carrier is not particularly limited. The carrier can be selected from materials commonly used in the art, or obtained by a conventional method, or commercially available in the present invention.

Some examples of pharmaceutically acceptable carriers are cellulose and its derivatives (such as methylcellulose, ethylcellulose, hydroxypropylmethylcellulose, sodium carboxymethylcellulose, etc.), gelatin, talc, solid lubricants (such as stearic acid, magnesium stearate), calcium sulfate, plant oil (such as soybean oil, sesame oil, peanut oil, olive oil, etc.), polyols (such as propylene glycol, glycerin, mannitol, sorbitol, etc.), emulsifier (such as Tween©), wetting agent (such as sodium lauryl sulfate), buffer agent, chelating agent, thickener, pH regulator, transdermal enhancer, colorant, flavoring agent, stabilizer, antioxidant, preservative, bacteriostatic agent, pyrogen-free water, liposomes, etc.

In addition to the active pharmaceutical ingredient, the liquid formulations can contain inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil or a mixture of these substances.

In addition to these inert diluents, the composition can also contain adjuvants such as wetting agents, emulsifiers and suspensions and the like.

In the present invention, the dosage form of the pharmaceutical composition contains (but is not limited to): oral preparation, injection, and external preparation. Representative examples contain (but are not limited to): tablets, injections, infusions, ointments, gels, solutions, microspheres, films.

A preferred dosage form is ophthalmic formulation. Typically, the ophthalmic preparation is eye drop, emulsion, gel, eye ointment, sustained release microsphere, intraocular sustained-release implant tablet, medicinal sustained-release film.

The ophthalmic formulation comprises a pharmaceutically acceptable pharmaceutical carrier, which representatively contain (but is not limited to): solvent or diluent, surfactant, thickener, osmotic pressure regulator, pH regulator, bacteriostatic agent, chelating agent.

Solvent

The solvent or diluent used in ophthalmic preparation (such as guttae ophthalmicae, eye drops) comprises aqueous solvent or non-aqueous solvent. The aqueous solvent comprises distilled water, physiological saline, water for injection, etc.; the non-aqueous solvent used comprises ethanol, propylene glycol, glycerin, plant oil (such as olive oil, castor oil, corn oil, soybean oil for injection), etc.

Surfactant

In ophthalmic formulations, the surfactant is selected from the group consisting of anionic surfactant, cationic surfactant, nonionic surfactant, chaotropic surfactant, and a combination thereof. Wherein the nonionic surfactant is selected from the group consisting of: Tween, Span, fatty glyceride, polyoxyethylene, polyoxyethylene-polyoxypropylene copolymer and a combination thereof. The amount (or content) of the surfactant is generally 0-2 wt %, more preferably 0.1-1 wt %.

Thickener

In ophthalmic preparations, the thickener can be used to increase the system viscosity, keep the system in a uniform and stable suspension state or emulsion state. The retention time of the drug in the eye can be increased by adding an appropriate amount of thickener, thus increasing the absorption of the effective small ingredients in the eye.

In the ophthalmic preparation, the thickener is preferably chitosan, hydroxypropylmethylcellulose (HPMC), methylcellulose (MC), and povidone (PVP), gelatin, sodium carboxymethylcellulose (CMC-Na), etc. Generally, The amount (or content) of the thickener is 0-6 wt %, preferably 0.1-5 wt %.

Osmotic Pressure Regulator

An appropriate osmotic pressure regulator is added to the phthalmic preparation to make the osmotic pressure of the ophthalmic preparation similar to the osmotic pressure of the human eye environment, thereby reducing irritation to the eye. Representatively, osmotic pressure regulator commonly used comprises (but is not limited to) acetic acid, sodium acetate, sodium bicarbonate. Generally, the amount (or content) of the osmotic pressure regulator make the ophthalmic formulation keep in an isosmotic or isotonic environment.

pH Regulator

An appropriate pH regulator is added to the phthalmic preparation to make the pH of the phthalmic preparation in an appropriate range, similar to the pH of the human eye environment, thereby reducing irritation to the eye. Representatively, pH regulator commonly used comprises (but is not limited to) sodium chloride, potassium chloride, and glucose. Generally, the amount (or content) of the pH regulator makes the pH of the ophthalmic preparation keep at 5-9.

Bacteriostatic Agent

In ophthalmic preparations, bacteriostatic agent can kill or inhibit the growth of bacteria in the cream, prevent bacterial overgrowth which endanger human health. In the present invention, the bacteriostatic agent is not particularly limited and can be one of nipagin alcohol or nipagin ester or a combination thereof. Representatively, the bacteriostatic agent of the present invention is selected from the group consisting of methylparaben, ethylparaben, propylparaben, and a combination thereof.

Chelating Agent

The stability of ophthalmic preparation can be increased by adding a certain amount of chelating agent such as EDTA to the ophthalmic preparation. Generally, the concentration of the chelating agent ranges from 0-0.05 wt %. The mode of administration of the composition or formulation of the present invention is not particularly limited, and representative mode of administration comprises (but is not limited to) local administration, oral administration, injection, etc.

A preferred mode of administration is the local administration of the composition or formulation to the eye, which includes (but is not limited to) conjunctiva, retrobulbar, periocular, retina, suprachoroidal or intraocular administration, etc. The representative mode is, for example, eye drops, intraocular injection, eye mucosa injection, eye mucosa coating, etc.

The pharmaceutical preparation should be matched with the mode of administration. The formulation of the present invention can also be used together with other synergistic therapeutic agents (including before, during or after use). When using pharmaceutical composition or preparation, a safe and effective dose of a drug is administered to a subject in need (e.g. human or non-human mammals). The safe and effective dose is usually at least 10 μg/kg body weight, and does not exceed about 8 mg/kg body weight in most cases, preferably the dose is about 10 μg/kg body weight to about 1 mg/kg body weight. Of course, the specific dose should also take into account the route of administration, patient health and other factors, which are within the ability of skilled physicians.

A Method for Non-Therapeutically and/or Non-Diagnostically Improving Lens Transparency In Vitro

A method for non-therapeutically improving or maintaining lens transparency in vitro is provided in the present invention, comprising contacting lens with the compound of formula I, or the optical isomer, racemate, solvate, pharmaceutically acceptable salt, prodrug, or deuterated compound thereof and/or inonotus obliquus extract to inhibit lens lesions, wherein the compound of formula I is as defined in the present invention.

In the method for non-therapeutically improving lens transparency in vitro, lens lesions comprise opacity or oxidation, etc., the inhibition of lens lesions comprise improving or maintaining lens transparency.

A Method for Preventing and/or Treating Eye Diseases

A method for preventing and/or treating eye diseases is also provided in the present invention, comprising administering the compound of formula I, or the optical isomer, racemate, solvate, pharmaceutically acceptable salt, prodrug, or deuterated compound thereof and/or inonotus obliquus extract to a subject in need.

In another preferred embodiment, the subject is a human and a non-human mammal. Representatively, the non-human mammal comprises (but is not limited to): pet (such as dog and cat), livestock (such as cattle, sheep, horse, pig), various zoo animals (pandas, elephants, etc.).

In another preferred embodiment, the subject further comprises other animals other than human and non-human mammals, such as non-mammals.

The Main Advantages of the Present Invention Include:

(a) The present inventors have firstly discovered the compound of the formula I and the inonotus obliquus extract can prevent and/or treat ocular diseases such as cataract prevention and treatment, and have a remarkable therapeutic effect.

(b) The compound of the formula I and the inonotus obliquus extract of the present invention have excellent safety with little or no toxic side effects.

(c) The compound of the formula I and the inonotus obliquus extract of the present invention have slow metabolism in the body's environment and high bioavailability, and are suitable for oral and local administration, etc.

(d) The compound of the formula I and the inonotus obliquus extract of the present invention have a good development and application prospect for the treatment of eye diseases.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacturer's instructions. Unless indicated otherwise, parts and percentage are calculated by weight. The experimental animals used in the example of the invention are all approved by the ethical committee at the location of the animal feeding base.

### Example 1

Inotodiol Eyedrop Formula:

TABLE 1
Inotodiol eyedrop formula in example 1

| Component | content |
|---|---|
| Hydroxypropyl-β-cyclodextrin | 4 g |
| Tween-80 | 10 mg |
| Inotodiol | 0.125 g |
| EDTA-2Na | 10 mg |
| PBS solution | constant volume to 10 mL |

Preparation Method

10 mg of Tween-80 and 0.125 g of inotodiol were sequentially added to 2 mL of PBS solution, and mixed uniformity by ultrasound to obtain composition A. Then 4 g of hydroxypropyl-β-cyclodextrin was added to 5 mL of PBS solution to obtain composition B. The composition B was added to composition A four times, and mixed uniformity by ultrasound, then 10 mg of EDTA-2Na was added, and PBS solution was added to constant volume to 10 mL.

### Comparative Example 1

Lanosterol Eyedrop Formula:

TABLE C1
Lanosterol eyedrop formula in comparative example C1

| Component | content |
|---|---|
| Hydroxypropyl-β-cyclodextrin | 4 g |
| Tween-80 | 10 mg |
| lanosterol | 0.125 g |
| EDTA-2Na | 10 mg |
| PBS solution | constant volume to 10 mL |

Preparation Method

10 mg of Tween-80 and 0.125 g of lanosterol were sequentially added to 2 mL of PBS solution, and mixed uniformity by ultrasound to obtain composition A. Then 4 g of hydroxypropyl-β-cyclodextrin was added to 5 mL of PBS solution to obtain composition B. The composition B was added to composition A four times, and mixed uniformity by ultrasound, then 10 mg of EDTA-2Na was added, and PBS solution was added to constant volume to 10 mL.

### Example 2

Trametenolic Acid Eyedrop Formula:

TABLE 2
Trametenolic acid eyedrop formula

| Component | content |
|---|---|
| Hydroxypropyl-β-cyclodextrin | 4 g |
| Tween-80 | 10 mg |
| trametenolic acid | 0.125 g |
| EDTA-2Na | 10 mg |
| PBS solution | constant volume to 10 mL |

Preparation Method

10 mg of Tween-80 and 0.125 g of trametenolic acid were sequentially added to 2 mL of PBS solution, and mixed uniformity by ultrasound to obtain composition A. Then 4 g of hydroxypropyl-β-cyclodextrin was added to 5 mL of PBS solution to obtain composition B. The composition B was added to composition A four times, and mixed uniformity by ultrasound, then 10 mg of EDTA-2Na was added, and PBS solution was added to constant volume to 10 mL.

### Comparative Example 2

Lanosterol Eyedrop Formula:

TABLE C2
eyedrop formula in comparative example C2

| Component | content |
|---|---|
| Hydroxypropyl-β-cyclodextrin | 4 g |
| Tween-80 | 10 mg |
| lanosterol | 0.125 g |
| EDTA-2Na | 10 mg |
| PBS solution | constant volume to 10 mL |

Preparation Method

10 mg of Tween-80 and 0.125 g of lanosterol were sequentially added to 2 mL of PBS solution, and mixed uniformity by ultrasound to obtain composition A. Then 4 g of hydroxypropyl-β-cyclodextrin was added to 5 mL of PBS solution to obtain composition B. The composition B was added to composition A four times, and mixed uniformity by ultrasound, then 10 mg of EDTA-2Na was added, and PBS solution was added to constant volume to 10 mL.

### Example 3 Investigation on the Therapeutic Effect of Karyotypic Cataract

In Example 3, the therapeutic effects of the two active ingredients of Example 1 and Comparative Example 1 on lens lesions (cataract) were compared.

3.1 Cataract Modeling

A rat cataract model is established. Wistar rats (10-13 days after birth, male or female) were selected and modeled with sodium selenite, and each rat was injected subcutaneously in the back and neck at a dose of 20 mol/kg body weight. Sodium selenite is formulated by using physiological saline, and the prepared solution is sterilized by a 0.22 m membrane and stored at room temperature away from light. Obvious karyotypic cataract was observed in the eyes of rats after opening their eyes.

3.2 Investigation on the Therapeutic Effect of Cataract

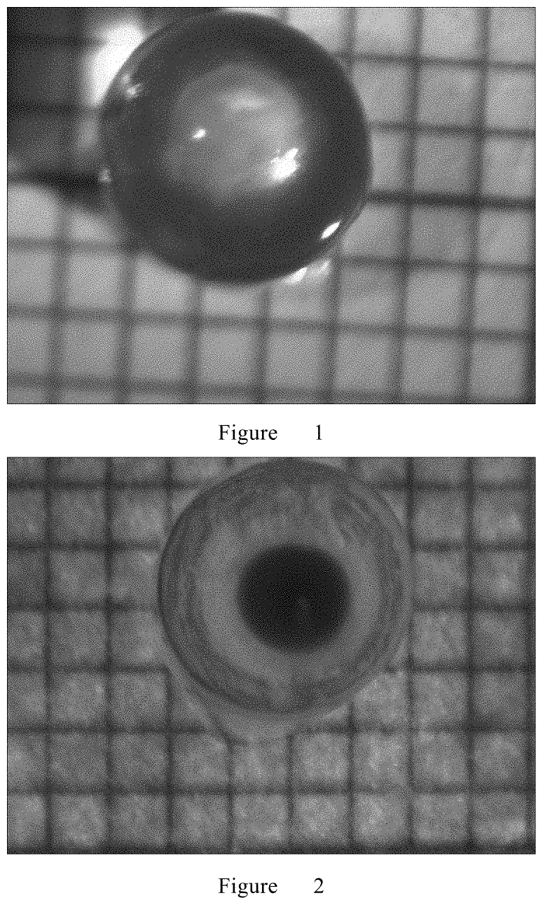

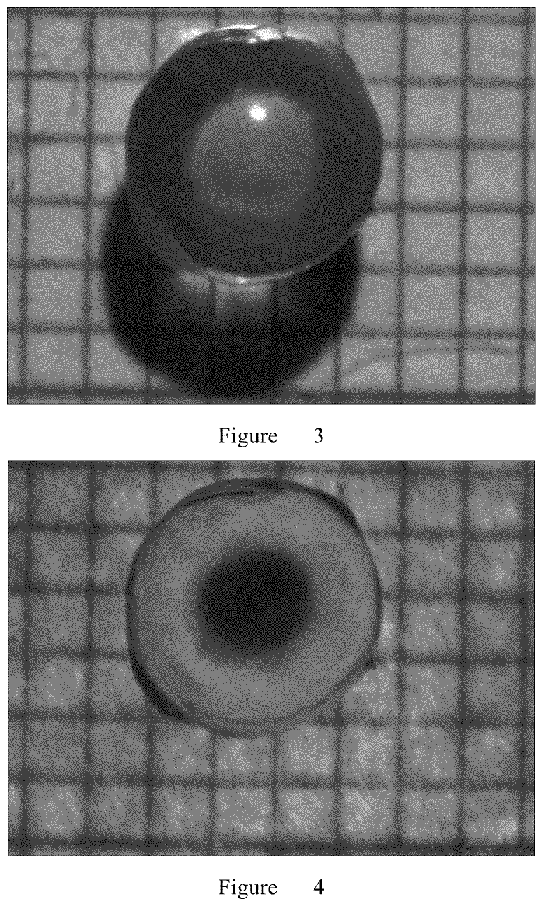

Eight cataract model rats were equally divided into two groups according to the left and right eye of each rat, and eight corresponding eye lens in each group were soaked with the formula of Example 1 (Example 1 group) and the formula of Comparative Example 1 (Comparative Example 1 group) respectively. Before the start of the experiment, the morphologies of the eye lens of the two groups of rats were observed and photographed, the morphologies of the upper and lower light source of a rat eye lens after successful cataract modeling in example 1 group were shown in FIG. 1 and FIG. 2, respectively, the morphologies of the upper and the lower light source of a rat eye lens after successful cataract modeling in Comparative example 1 group were shown in FIG. 3 and FIG. 4, respectively, all the rats lens were taken out, and all photographed scores were 5 grade.

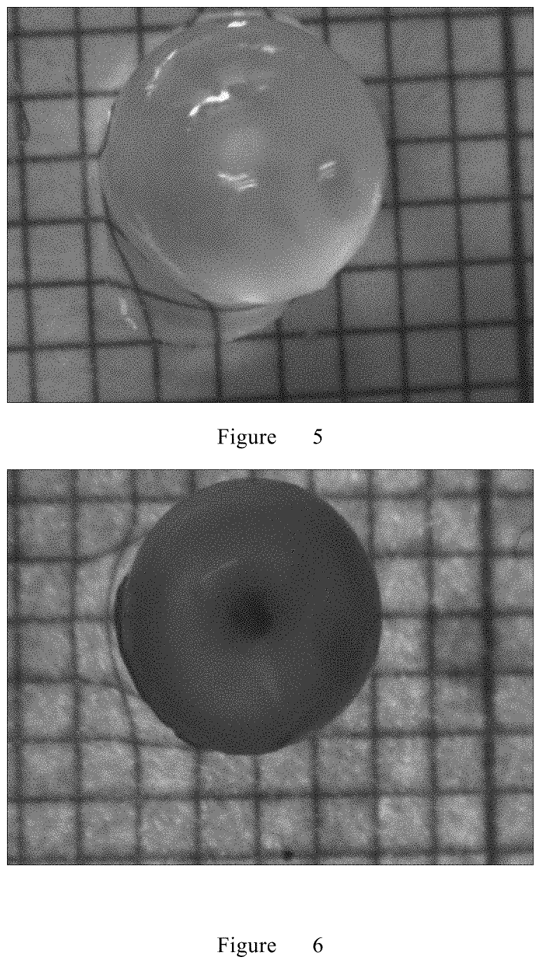

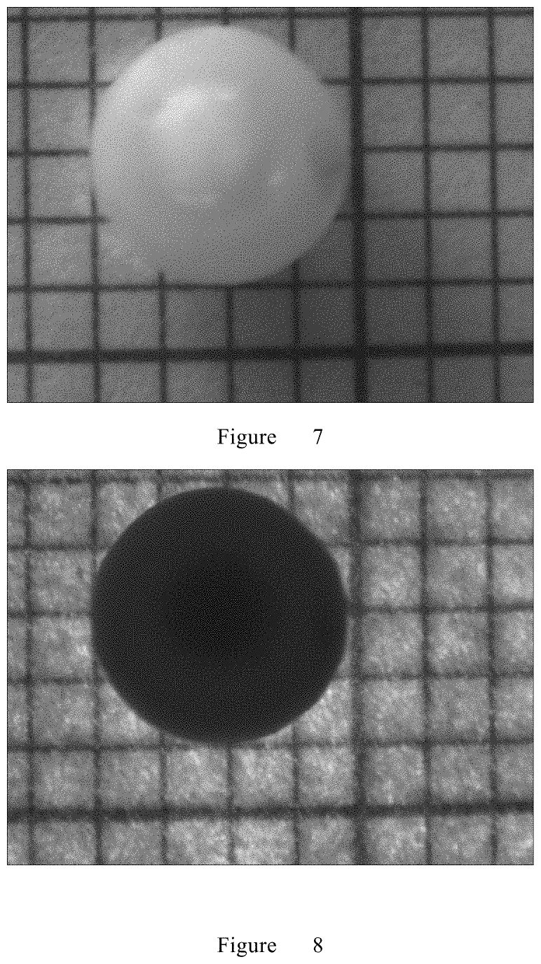

The left and right eye lens of the rats in Example 1 group and Comparative Example 1 group were immersed in the inotodiol eyedrop and the structural analogue lanosterol eyedrop with the same concentration, respectively, and the effect was observed and scored two weeks later, the morphologies of the upper and lower light source of a rat eye lens after the end of the experiment in example 1 group were shown in FIG. 5 and FIG. 6; the morphologies of the upper and lower light source of a rat eye lens after the end of the experiment in comparative example 1 group were shown in FIG. 7 and FIG. 8.

Score Standard

5 grade—the cataract nucleus is plump, the cataract edge is smooth, and the cataract is completely opaque;

4 grade—the nucleus center of cataract is plump, the cataract edge appears diffusion, and the cataract is completely opaque;

3 grade—the volume of cataract nucleus is decreased but not significantly, and the density of opaque area decreases;

2 grade—the cataract core is shrunken significantly;

1 grade—the cataract nucleus disappears, the cortex is transparent, and the lens returns to normal.

The results were as follows:

TABLE 3
The scores of therapeutic effect of Example 1 and Comparative
Example 1 on cataract

| | group | | | | | | | |
|---|---|---|---|---|---|---|---|---|
| | Rat A | Rat B | Rat C | Rat D | Rat E | Rat F | Rat G | Rat H |
| Example 1 | 2 | 2 | 3 | 2 | 3 | 3 | 3 | 2 |
| Comparative | 5 | 4 | 5 | 3 | 5 | 4 | 5 | 4 |
| Example 1 | | | | | | | | |

The table 3 shows that compared to the structural analogue lanosterol, the compound of formula I (the compound having O or S group at a specific position) in the present invention, represented by inotodiol, also has excellent therapeutic effect on cataract even with simple administration means, and can quickly alleviate, cure and prevent animal cataract.

### Example 4

In Example 4, the therapeutic effects of the two active ingredients of Example 2 and Comparative Example 2 on lens lesions (cataract) were compared.

4.1 Cataract Modeling

A rat cataract model is established. Wistar rats (10-13 days after birth, male or female) were selected and modeled with sodium selenite, and each rat was injected subcutaneously in the back and neck at a dose of 20 μmol/kg body weight. Sodium selenite is formulated by using physiological saline, and the prepared solution is sterilized by a 0.22 m membrane and stored at room temperature away from light. Obvious karyotypic cataract was observed in the eyes of rats after opening their eyes.

4.2 Investigation on the Therapeutic Effect of Cataract

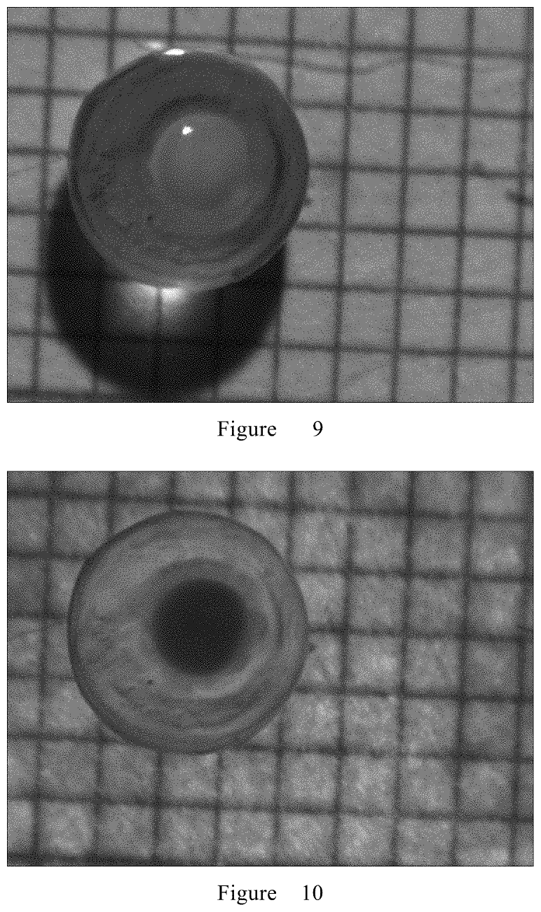

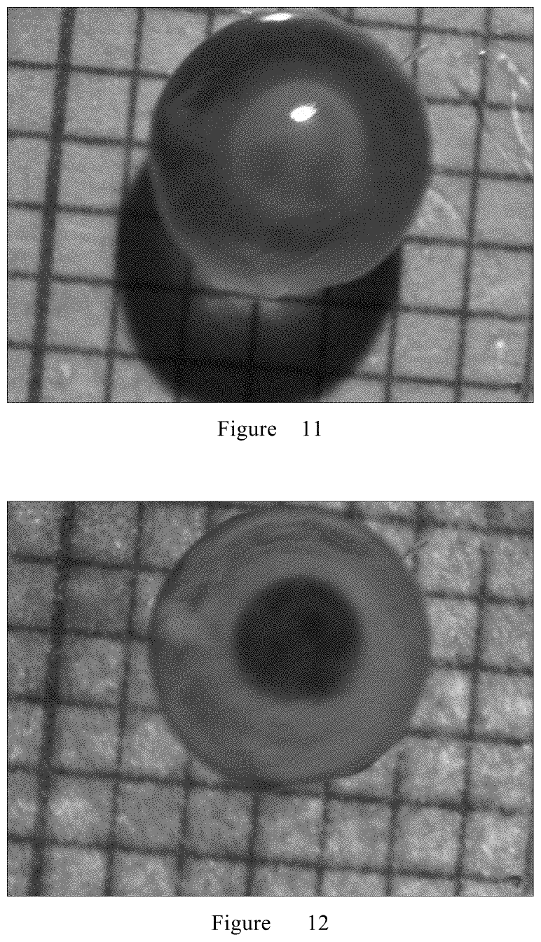

Eight cataract model rats were equally divided into two groups according to the left and right eye of each rat, and eight corresponding eye lens in each group were treated with the formula of Example 2 (Example 2 group) and the formula of Comparative Example 2 (Comparative Example 2 group) respectively. Before the start of the experiment, the morphologies of the eye lens of the two groups of rats were observed and photographed, the morphologies of the upper and lower light source of a rat eye lens after successful cataract modeling in example 2 group were shown in FIG. 9 and FIG. 10, respectively, the morphologies of the upper and the lower light source of a rat lens after successful cataract modeling in Comparative example 2 were shown in FIG. 11 and FIG. 12, respectively, all the rats lens were taken out, and all photographed scores were 5 grade.

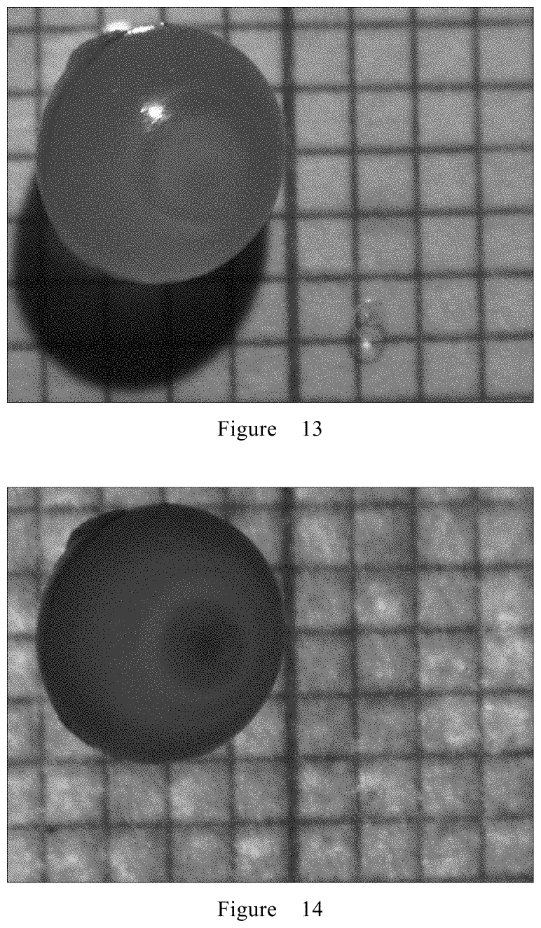

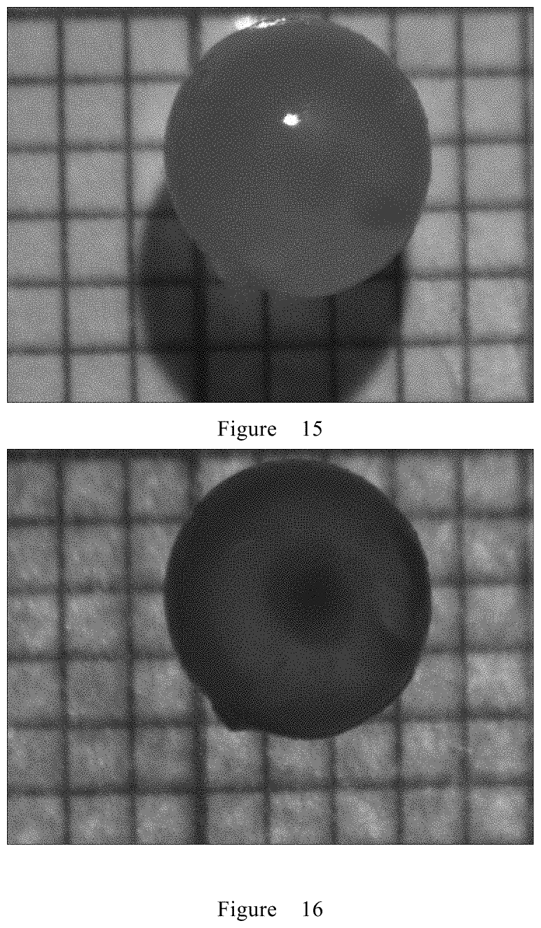

The left and right eye lens of the rats in Example 2 group and Comparative Example 2 group were immersed in the trametenolic acid eyedrop and the structural analogue lanosterol eyedrop with the same concentration, respectively, and the effect was observed and scored 9 days later (Table 4). After the lens were continuously immersed for three weeks, the morphologies of the upper and lower light source of a rat eye lens after the end of the experiment in example 2 group were shown in FIG. 13 and FIG. 14; the morphologies of the upper and lower light source of a rat eye lens after the end of the experiment in comparative example 2 group were shown in FIG. 15 and FIG. 16;

The score standard was the same as that in Example 3.

The results were as follows:

TABLE 4
The scores of therapeutic effect of Example 2 and Comparative
Example 2 on cataract

| | group | | | | | | | |
|---|---|---|---|---|---|---|---|---|
| | Rat A | Rat B | Rat C | Rat D | Rat E | Rat F | Rat G | Rat H |
| Example 2 | 3 | 3 | 3 | 2 | 4 | 2 | 3 | 2 |
| Comparative | 5 | 5 | 5 | 5 | 5 | 4 | 5 | 5 |
| Example 2 | | | | | | | | |

The table 4 and FIG. 9-16 show that compared to lanosterol, the compound of formula I (the compound having O or S group at a specific position) in the present invention, represented by trametenolic acid, also has excellent therapeutic effect (the opaque area in the center of karyotypic cataract lens is obviously decreased after treatment with trametenolic acid eyedrop) on cataract even with simple administration means, and can quickly alleviate, cure and prevent animal cataract.

### Example 5 Pharmacokinetic Study

Experimental Methods and Results

SD rats (about 10 weeks old, male or female) were anesthetized by intraperitoneal injection of pentobarbital sodium and pupil was dilated, then eye glass cavity injection surgery was performed. All animals were injected with the same kind of drugs in both eyes. The amount of drug injected into each glass cavity was 5 μl, and the number of test animals per drug formulation (Example 1, Example 2 and Comparative Example 1) was four, and the drug metabolism in the aqueous humor was detected 10 days after the surgery.

Compared with the initial concentration of the drug immediately after injection, the concentration of lanosterol decreased by 82%±14.7%, while the concentration of inotodiol decreased by 11%±2.3%, and the concentration of trametenolic acid decreased by 8%±1.5%.

The above results suggested that for the eye diseases related to lens lesions, both inotodiol and trametenolic acid had more excellent pharmacokinetic properties and could maintain the concentration required for disease treatment or prevention in aqueous humor for a long time.

### Example 6

Inotodiol Eye Ointment Formula and Preparation Method:

TABLE 5
Inotodiol eye ointment formula

| Component | content |
|---|---|
| Inotodiol | 1.25 g |
| White vaselin | 75 g |
| Liquid paraffin | 12.5 g |

Preparation Method

1.25 g of inotodiol, 75 g of white vaselin and 12.5 g of liquid paraffin were weighed and placed in a container and sonicated in a water bath at 70° C. until all the component were uniformly dispersed, and transferred quickly to a clean, sterile eye tube, cooled quickly in ice bath until the ointment was solidified and then stored at 4° C.

### Example 7

Trametenolic Acid Eye Ointment Formula and Preparation Method:

TABLE 6
Trametenolic acid eye ointment formula

| Component | content |
|---|---|
| Trametenolic acid | 1.25 g |
| White vaselin | 75 g |
| Liquid paraffin | 12.5 g |

Preparation Method

1.25 g of trametenolic acid, 75 g of white vaselin and 12.5 g of liquid paraffin were weighed and placed in a container and sonicated in a water bath at 70° C. until all the component were uniformly dispersed, and transferred quickly to a clean, sterile eye tube, cooled quickly in ice bath until the ointment was solidified and then stored at 4° C.

### Comparative Example 3

Blank Eye Ointment Formula and Preparation Method:

TABLE C3
Blank eye ointment formula

| Component | content |
|---|---|
| White vaselin | 75 g |
| Liquid paraffin | 12.5 g |

Preparation Method

75 g of white vaselin and 12.5 g of liquid paraffin were weighed and placed in a container and sonicated in a water bath at 70° C. until all the component were uniformly dispersed, and transferred quickly to a clean, sterile eye tube, cooled quickly in ice bath until the ointment was solidified and then stored at 4° C.

### Comparative Example 4

Lanosterol Eye Ointment Formula and Preparation Method:

TABLE C4
Lanosterol eye ointment formula

| Component | content |
|---|---|
| Lanosterol | 1.25 g |
| White vaselin | 75 g |
| Liquid paraffin | 12.5 g |

Preparation Method

1.25 g of lanosterol, 75 g of white vaselin and 12.5 g of liquid paraffin were weighed and placed in a container and sonicated in a water bath at 70° C. until all the component were uniformly dispersed, and transferred quickly to a clean, sterile eye tube, cooled quickly in ice bath until the ointment was solidified and then stored at 4° C.

### Comparative Example 5

Low-Dose Lanosterol Eye Ointment Formula and Preparation Method:

TABLE C4
Lanosterol eye ointment formula

| Component | content |
|---|---|
| Lanosterol | 0.45 g |
| White vaselin | 75 g |
| Liquid paraffin | 12.5 g |

Preparation Method

0.45 g of lanosterol, 75 g of white vaselin and 12.5 g of liquid paraffin were weighed and placed in a container and sonicated in a water bath at 70° C. until all the component were uniformly dispersed, and transferred quickly to a clean, sterile eye tube, cooled quickly in ice bath until the ointment was solidified and then stored at 4° C.

### Example 8

In Example 8, the primate cynomolgus monkeys were used as an animal model to investigate the therapeutic effect of inotodiol eye ointment in Example 6 on congenital cataract.

Investigation on Administration Mode and Therapeutic Effect of Congenital Cataract Animals

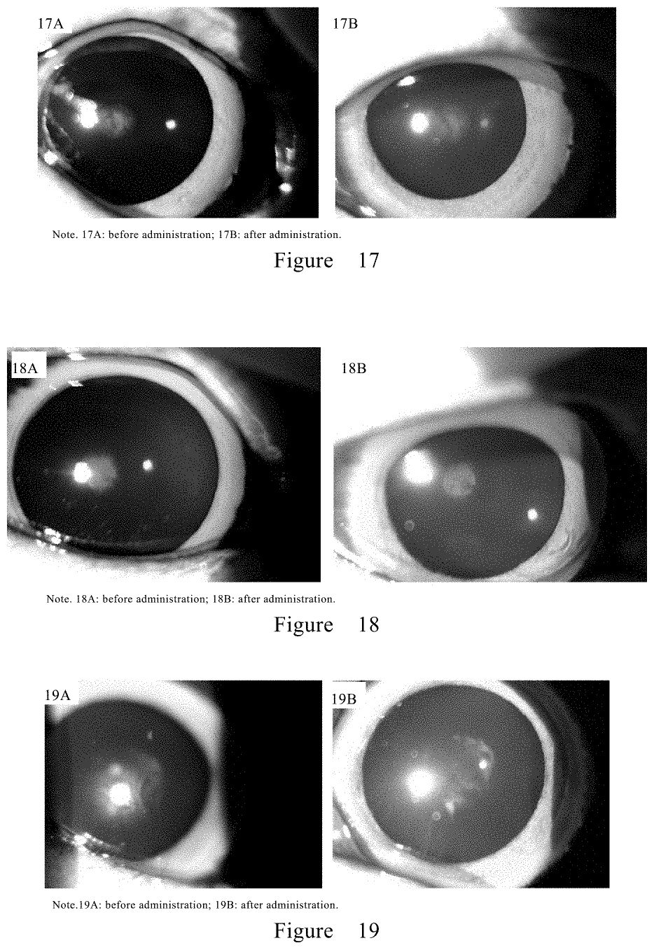

Cynomolgus monkeys with natural congenital cataract were selected and anesthetized, both eyes were detected by slit lamp and photographed to exclude other abnormalities or inflammatories in the eye after pupil was dilated. Both eyes were administrated simultaneously at 9:00 a.m., 13:00 p.m., and 17:00 p.m. daily. The administration mode was that the inotodiol eye ointment in Example 6 was directly applied to the corneal surface of the left and right eyeballs of the cynomolgus monkey, and the cynomolgus monkeys faced up when administered and the cynomolgus monkeys were held for 3-5 minutes after administration. After 95 days of continuous administration of left and right eyes, the cynomolgus monkeys were re-anesthetized, the pupil was dilated, and both eyes were detected by slit lamp and photographed. The morphologies of left and right eyes of cynomolgus monkey before and after the inotodiol eye ointment administration were shown in FIG. 17 and FIG. 18. As could be seen from the FIG. 17 and FIG. 18, the contrast pictures were taken with approximately the same slit lamp inspection condition after the eyes were treated with the inotodiol eye ointment, the degree of cataract turbidity of the left and right eye lens of the tested cynomolgus monkeys was significantly decreased, and the turbidity was disappeared completely in partial turbid area before administration, the turbid area continued to decrease, no new turbid area appeared in the whole lens. The light transmittance of the congenital cataract area in the original lens core polarity position was significantly increased, the lens of the left and right eyes was significantly restored to a transparent (normal) state. This phenomenon demonstrates for the first time that the compound inotodiol can reverse the turbidity of congenital cataracts of primates and that the mode of administration throughout the treatment is non-invasive. The above results indicate that inotodiol has an obvious therapeutic effect on congenital cataracts of primates.

### Example 9

In Example 9, the aged primate cynomolgus monkeys were used as an animal model to investigate the therapeutic effect of inotodiol eye ointment in Example 6 on spontaneous cataract, Comparative example 4 was used as control group at the same time. The monkeys used in all experiments were males.

Investigation on Therapeutic Effect of Spontaneous Cataract

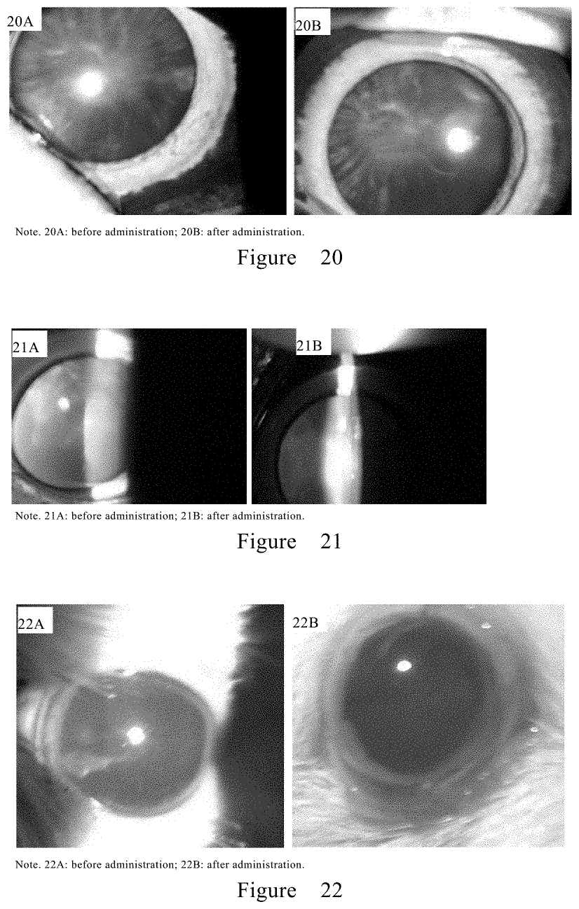

After a 17-year-old cynomolgus monkey (No. 013321) was anesthetized and the pupil was dilated, the eyes was detected by slit lamp and photographed to exclude other eye diseases, the right eye was normal and had no cataract, the left eye was suboccipital cortical opacity in the central lower pupil area, which belongs to spontaneous cataract. After a 19-year-old monkey (No. 990447) pupil was dilated and the eyes was detected by slit lamp and photographed to exclude other eye diseases, both the left and right eyes were posterior subcortical turbidty, which belongs to spontaneous cataract. The blood glucose levels of two monkeys were within the normal range, and two monkeys had no history of diabetes and ocular trauma. Drugs were administered three times a day at 9:00 a.m., 13:00 p.m., and 17:00 p.m. The administration mode was that the inotodiol eye ointment in Example 6 was directly applied to the left diseased eyeball corneal surface of monkeys numbered 013321 and 90447 respectively. The monkeys faced up when administered and the monkeys were held for 3-5 minutes after administration. After 42 days of continuous administration of left diseased eyeball of two monkeys, the left eyeballs were checked again in the same way and photographed. At the same time, a 19-year-old monkey (No. 993747) with spontaneous cataract was used as control group. The lanosterol eye ointment in Comparative example 4 was used for the same administration, examination, and photographing. The eye results before and after the administration of inotodiol eye ointment were shown in FIG. 19 and FIG. 20. The FIG. 19 and FIG. 20 showed that after the treatment with the inotodiol eye ointment, the degree of lens turbidity of diseased eyes in the aged monkey was significantly decreased, and the light transmittance of severe areas of the original cataract opacity was significantly increased, and even multiple parts have been completely restored transparency, the turbidity area of the entire lens cataract area was also significantly decreased, and the grade of lens cataract with drug treatment reversed to transparent (normal) level. The therapeutic effect of lanosterol in the control group was shown in FIG. 21 (the left eye of the same subject administered). It can be clearly observed from the comparison pictures before and after administration that lanosterol did not slow down the progression of spontaneous cataract in aged primates, and compared with before treatment, the turbid area inside the lens was increased by nearly 50% after lanosterol treatment, and the light transmittance of the whole lens was further decreased. The above results suggested that inotodiol had an obvious and unique therapeutic effect on spontaneous cataract in aged primates.

### Example 10

In Example 10, the rats were used as an animal model to investigate the therapeutic effect of inotodiol eye ointment in Example 6 and trametenolic acid eye ointment in Example 7 on traumatic cataract, the blank eye ointment in Comparative example 3 was used as blank control, and the lanosterol eye ointment in Comparative example 4 was used as the positive control group at the same time.

Investigation on Therapeutic Effect of Traumatic Cataract

Sixteen SD rats (4-6 weeks, half males and half females) were divided into four groups, and each group contained 2 male and 2 female SD rats (Rats in each group were numbered A, B, C and D, in which A and B were male and C and D were female). After each group of SD rats were anesthetized with sodium pentobarbital, the pupil was dilated, the cornea and phacocyst were punctured with an injection needle, and the needle repeatedly slided inside the lens cortex under microscope observation until obvious turbidity appeared in the lens. Two weeks after the end of the modeling operation, the rats were re-anesthetized, the pupil was dilated and photographed, and the traumatic cataract and its degree were evaluated. The lens cortex was observed with a slit lamp, and the scoring evaluation was based on the lens opacities classification system II (LOCS II) standard. Because of the local lens cataract caused by trauma, the turbid area and other factors were ignored when scoring at the first time and only the degree of cortical turbidity was examined. Later, the efficacy was evaluated by the changes in the depth degree of cortical turbidity. After the first photograph, the drug was applied the next day, and four groups of SD rats were given inotodiol eye ointment in Example 6 (experimental group 1), trametenolic acid eye ointment in Example 7 (experimental group 2), blank eye ointment in Comparative example 3 (solvent control group 3), and lanosterol eye ointment in Comparative Example 4, respectively. The drugs were continuously administered on working days, and not administered on non-working days, the administration frequency was once a day on working days, half a mung bean of drug was directly applied to the cornea of each rat. Three months later, the rats were re-anesthetized, the pupil was dilated, photographed (except the solvent control group 3), the traumatic cataract and its extent in rats were evaluated. The results of therapeutic effect of inotodiol eye ointment in Example 6, trametenolic acid eye ointment in Example 7, blank eye ointment in Comparative example 3, and lanosterol eye ointment in Comparative Example 4 on traumatic cataract were shown in Table 7-10 and FIG. 22-24.

TABLE 7
The therapeutic effect of inotodiol eye ointment in Example 6 on
traumatic cataract of rats

| | Experimental group 1 | | | | | | | |
|---|---|---|---|---|---|---|---|---|
| | Left | Right | Left | Right | Left | Right | Left | Right |
| | eye of | eye of | eye of | eye of | eye of | eye of | eye of | eye of |
| | rat A | rat A | rat B | rat B | rat C | rat C | rat D | rat D |
| Before | CV | CIV | CI | CI | Ctr | CIII | CII | CII |
| administration | | | | | | | | |
| After | CII | CIV | Ctr | Ctr | C0 | CI | Ctr | C0 |
| administration | | | | | | | | |

TABLE 8
The therapeutic effect of trametenolic acid eye ointment in Example 7 on
traumatic cataract of rats

| | Experimental group 2 | | | | | | | |
|---|---|---|---|---|---|---|---|---|
| | Left | Right | Left | Right | Left | Right | Left | Right |
| | eye of | eye of | eye of | eye of | eye of | eye of | eye of | eye of |
| | rat A | rat A | rat B | rat B | rat C | rat C | rat D | rat D |
| Before | CII | CII | CI | Ctr | Ctr | CII | CIII | CII |
| administration | | | | | | | | |
| After | CII | Ctr | Ctr | Ctr | C0 | CI | Ctr | CI |
| administration | | | | | | | | |

TABLE 9
The therapeutic effect of blank eye ointment in Comparative example 3 on
traumatic cataract of rats

| | Solvent control group 3 | | | | | | | |
|---|---|---|---|---|---|---|---|---|
| | Left | Right | Left | Right | Left | Right | Left | Right |
| | eye of | eye of | eye of | eye of | eye of | eye of | eye of | eye of |
| | rat A | rat A | rat B | rat B | rat C | rat C | rat D | rat D |
| Before | CI | CII | CI | CII | Ctr | Ctr | CI | Ctr |
| administration | | | | | | | | |
| After | CI | CI | CI | CII | Ctr | Ctr | CI | CI |
| administration | | | | | | | | |

TABLE 10
The therapeutic effect of lanosterol eye ointment in Comparative
Example 4 on traumatic cataract of rats

| | Lanosterol | | | | | | | |
|---|---|---|---|---|---|---|---|---|
| | Left | Right | Left | Right | Left | Right | Left | Right |
| | eye of | eye of | eye of | eye of | eye of | eye of | eye of | eye of |
| | rat A | rat A | rat B | rat B | rat C | rat C | rat D | rat D |
| Before | CIV | CIII | Ctr | Ctr | CII | CI | CV | CIII |
| administration | | | | | | | | |
| After | CIV | CIII | C0 | C0 | CI | Ctr | CV | CIII |
| administration | | | | | | | | |

FIG. 22 clearly showed that the cortical turbidity area of eyes with the traumatic cataract completely restored transparency after administration of inotodiol eye ointment.

FIG. 23 clearly showed that the cortical turbidity area of eyes with the traumatic cataract almost restored transparency after administration of trametenolic acid eye ointment.

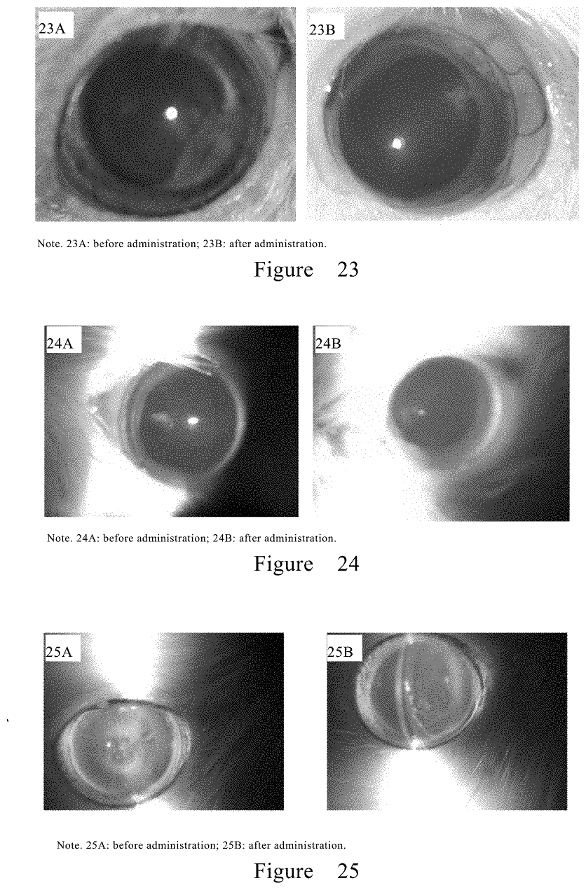

From FIG. 24 and Table 10, it could be concluded that in the treatment of traumatic cataract, lanosterol could restore transparency of lens with slight turbidity, but no obvious reversal of the disease has been observed in severe cataract.

In summary, Table 7-10 and FIG. 22-24 clearly showed that compared with lanosterol, both inotodiol and trametenolic acid had more excellent therapeutic effects on traumatic cataract, especially on the eyes with severe cataract.

### Example 11

In Example 11, the rats were used as an animal model to investigate the therapeutic effect of inotodiol eye ointment in Example 6 and trametenolic acid eye ointment in Example 7 on metabolic cataract in diabetic animal model, the blank eye ointment in Comparative example 3 was used as blank control, and the lanosterol eye ointment in Comparative example 4 was used as positive control at the same time. Since the commercial pirenoxine sodium eyedrop (BaiNeiTing) was an ophthalmic drug for the treatment of mild diabetic cataract or complicated cataract, so it was used as a positive control.

Investigation on Therapeutic Effect of Diabetic Metabolite Cataract

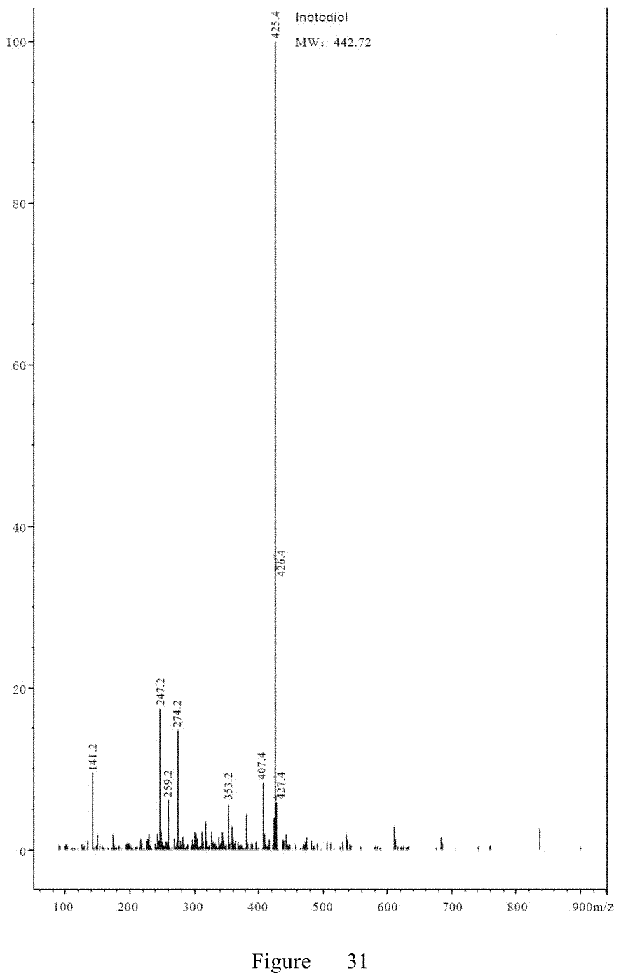

Twelve SD rats (8 weeks old) were divided into four groups, each group had three SD rats (Rats in each group were numbered A, B, C and D). At the same time, an 8-weeks male SD rat was added as the treatment subject of pirenoxine sodium eyedrop group after successful establishment of diabetic model. All diabetic rat models were established by intraperitoneal injection of streptozotocin (STZ). The tail vein blood was collected 2 weeks after the last injection, and the blood glucose levels of all rats were determined to be above 25.4 mmol/L, which was significantly higher than that of normal rats (6.6-9.7 mmol/L). All 13 rats were anesthetized with pentobarbital sodium after 2 months of model induction, the pupil was dilated and photographed, and the rat right eye cataract and its degree were evaluated. Right eye lens cortex of cataract animals was observed with a slit lamp, and the scoring evaluation was based on the lens opacities classification system II (LOCS II) standard (no score was evaluated in the pirenoxine sodium eyedrop group). After the first photograph, the drug was applied the next day, and four groups of SD rats were given inotodiol eye ointment in Example 6 (experimental group 1), trametenolic acid eye ointment in Example 7 (experimental group 2), blank eye ointment in Comparative example 3 (solvent control group 3), and lanosterol eye ointment in Comparative Example 4 (positive control group), respectively. The drugs were continuously administered on working days, and not administered on non-working days, the administration frequency was once a day on working days, half a mung bean of drug was directly applied to the rat cornea, and no drug was given to the left eye. The right eye of rat in the pirenoxine sodium eyedrop group was administered according to the administration mode and dosage of manufacturer's instructions (no administration on non-working days), and no drug was given to the left eye. All animal models were anesthetized again after 2 months of treatment, the blood of the rats were taken to measure blood sugar value (all above 18.6 mmol/L), the eyes were photographed to evaluate the degree of cataract in the lens and cortex of the right eye in rats. The results of therapeutic effect of inotodiol eye ointment in Example 6, trametenolic acid eye ointment in Example 7, blank eye ointment in Comparative example 3, and lanosterol eye ointment in Comparative Example 4 on diabetic metabolite cataract were shown in Table 11-14 and FIG. 25-28, and the therapeutic effect of pirenoxine sodium eyedrop group on rat right eye was shown in FIG. 29.

TABLE 11
The therapeutic effect of inotodiol eye ointment in Example 6
on diabetic metabolite cataract of rats

| Experimental | | | |
|---|---|---|---|
| group 1 | Rat A | Rat B | Rat C |
| Before | CII | CII | Ctr |
| administration | | | |
| After | Ctr | Ctr | Ctr |
| administration | | | |

TABLE 12

| The therapeutic effect of trametenolic acid eye ointment in | | | |
|---|---|---|---|
| Example 7 on diabetic metabolite cataract of rats | | | |
| Experimental | | | |
| group 1 | Rat A | Rat B | Rat C |
| Before | CI | Ctr | Ctr |
| administration | | | |
| After | Ctr | CI | Ctr |
| administration | | | |

TABLE 13
The therapeutic effect of blank eye ointment in Comparative
example 3 on diabetic metabolite cataract of rats

| Experimental | | | |
|---|---|---|---|
| group 1 | Rat A | Rat B | Rat C |
| Before | CI | Ctr | Ctr |
| administration | | | |
| After | CIII | CII | CII |
| administration | | | |

TABLE 14
The therapeutic effect of lanosterol eye ointment in Comparative
Example 4 on diabetic metabolite cataract of rats

| Experimental | | | |
|---|---|---|---|
| group 1 | Rat A | Rat B | Rat C |
| Before | CII | CI | Ctr |
| administration | | | |
| After | CIII | CI | CIII |
| administration | | | |

FIG. 25 clearly showed that the cortical turbid area of the diabetic metabolite cataract eyes was reduced in a large extent after administration of inotodiol eye ointment, and the degree of turbidity was also very shallow, and the entire lens almost restored transparent state.

FIG. 26 clearly showed that the cortical turbid area of the diabetic metabolite cataract eyes was reduced in a large extent after administration of trametenolic acid, and the degree of turbidity was also very shallow, and the entire lens almost restored transparent state.

FIG. 27 clearly showed that the turbid area of rat lens was increased after lanosterol administration, and turbidity also appeared in the area where no cataract was originally present, and the overall light transmission of the lens continued to decrease, and the condition was deteriorated. Lanosterol had no effect on reversing and restoring lens transparency for metabolic cataract induced by diabetes.

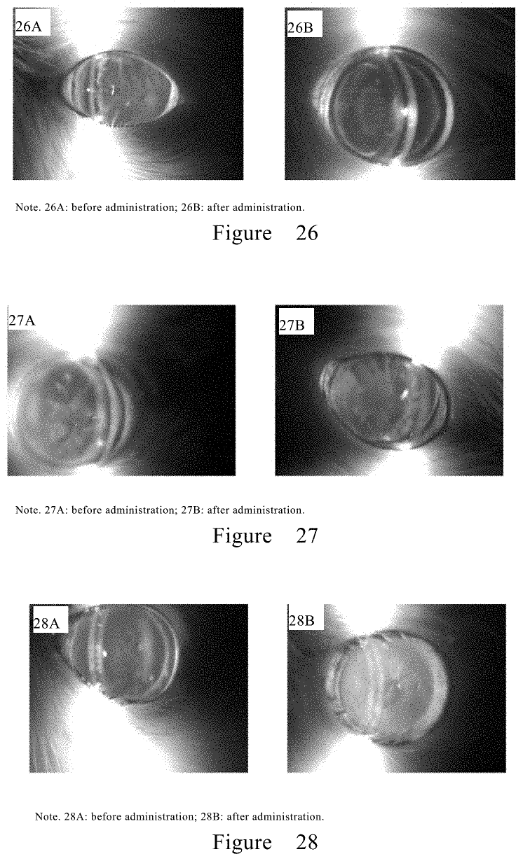

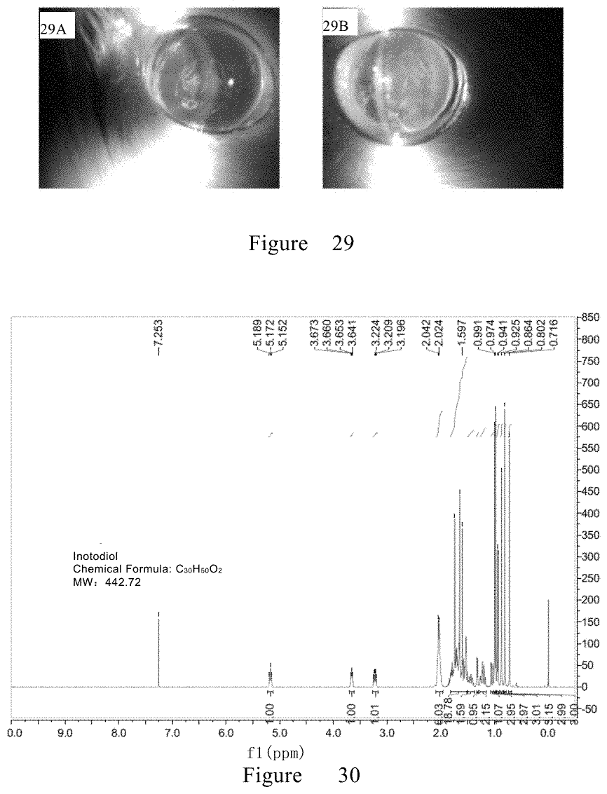

FIG. 28 showed that the degree of cataract in the diabetic rat model of the blank eye ointment control group was significantly deteriorated over time, the turbidity range was increased, and the degree of turbidity was aggravated.

FIG. 29 showed that the cataract in the diabetic rat model of the pirenoxine sodium eyedrop control group was further deteriorated over time, the overall turbidity of the lens was increased, and the degree of turbidity was increased, but the progress of the disease was better than the blank eye ointment control group.

Table 11-14 and FIG. 25-29 clearly showed that compared with lanosterol and commercial pirenoxine sodium eyedrop, both inotodiol and trametenolic acid not only had significant inhibitory effect on the development of diabetic metabolite cataract, but also had significant effect on restoring cataract lens transparency (reversal effect).

### Example 12

In Example 12, the dogs were used as an animal model to investigate the therapeutic effect of inotodiol eye ointment in Example 6 on senile cataract.

Investigation on Therapeutic Effect of Senile Cataract

A 13-year-old dog (Golden Retriever) eyes were detected by slit lamp after pupil was dilated. After confirming that the test animals had no other eye diseases and blood sugar level was normal, the left and right eyes were determined to be senile cataracts according to the lens Opacities Classification System II (LOCS II) standard, and cortical cataract rating scores were CIV and CIII. Both eyes weres simultaneously administered with inotodiol eye ointment in Example 6 once in the morning and evening of each day, twice a day, two soybeans of the ointment was directly applied to the cornea, the animal's head was fixed for 5 minutes after administration to increase the drug utilization rate. After 90 days of continuous administration, the lens was examined again in the same way and the degree of cortical cataract was evaluated. It was found that the left and right eyes cataracts were decreased to CI and CI, respectively. The results showed that inotodiol had significantly therapeutic effect on senile cataract.

### Example 13

Preparation of Inonotus Obliquus Extract 1

Inonotus obliquus extract 1:1 kg of the crude powder of dried sporophore of inonotus obliquus was refluxed with 95% ethanol at a ratio of material to liquid 1:75 (g: ml), and the extract solution was distilled under reduced pressure to remove solvent to obtain 40 g of ethanol extract. All ethanol extract was suspended in water and extracted with petroleum ether, the extract solution was separately distilled under reduced pressure to remove solvent to obtain 1.25 g of inonotus obliquus extract 1.

### Example 14

Preparation of Inonotus Obliquus Extract 2

Inonotus obliquus extract 2: The inonotus obliquus extract 1 prepared in Example 13 was subjected to silica gel column chromatography, and eluted with a gradient petroleum ether-ethyl acetate (15:1-2:1), 50 mL of eluent was collected each time, the same fractions were combined according to the results of thin-layer chromatography, and the single component was recrystallized after the solvent was removed by distillation under reduced pressure, and the inonotus obliquus extract 2 was obtained by eluting with petroleum ether-ethyl acetate 5:1.

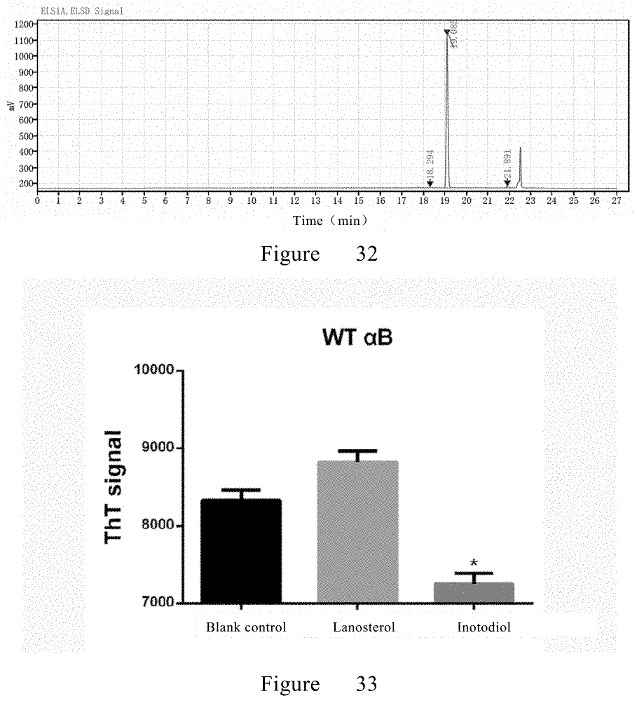

Nuclear magnetic resonance (NMR), mass spectrometry (MS) and high performance liquid chromatography (HPLC) of inonotus obliquus extract 2 were shown in FIG. 30-32, and the analysis results of NMR, MS and HPLC showed the inonotus obliquus extract 2 was a single inotodiol component with a purity of 98.36%. The structural formula of the inonotol was as follows:

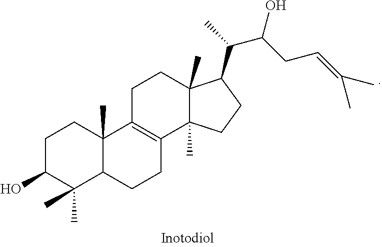

### Example 15

Preparation of Extract Ophthalmic Preparation

In the present example, the extract 1 in Example 13 was used as an active ingredient, and eye ointment was prepared by conventional method. The formula was follows:

Inonotus Obliquus Extract 1 Eye Ointment

| | |
|---|---|
| Extract 1 | 1.25 g |
| White vaselin | 75 g |
| Liquid Paraffin | 12.5 g |

Preparation Method

1.25 g of extract 1, 75 g of white vaselin and 12.5 g of liquid paraffin were weighed and placed in a container and sonicated in a water bath at 70° C. until all the component were uniformly dispersed, and then transferred quickly to a clean, sterile eye tube, cooled quickly in ice bath until the ointment was solidified and then stored at 4° C.

### Example 16

Therapeutic Effect of Inonotus Obliquus Extract on Acquired Cataract (Non-Congenital Cataract)

In the present example, the pharmaceutical activities of the ophthalmic preparations prepared by the extracts in example 13 and example 14 were tested.

According to published literature data, a small amount of lanosterol was present in inonotus obliquus extract 1, and its mass fraction accounted for about 16%. Because the effect of lanosterol on the treatment of cataract was positively correlated with the dosage of the drug, in order to eliminate the interference of a small amount of lanosterol on the efficacy of inonotus obliquus extract on the treatment of cataract. In the experimental design, besides demonstrating the efficacy of inonotus obliquus extract eye ointment (Example 15), a group of low dose lanosterol eye ointment control group was added (Comparative Example 5) to demonstrate whether lanosterol with dosage more than 2 times that of inonotus obliquus extract 1 could significantly treat cataract in animals. Meanwhile, lanosterol (Comparative Example 4) and inotodiol (Comparative Example 6) were designed as control group and experimental group.

Eight dogs of Golden Retriever and Teddy with natural eye cataracts were selected. After eliminating other eye diseases, the left and right eye lens were observed and scored by dilatation and slit lamp. After random grouping, every two dogs corresponded to one pharmaceutical formula (Comparative Example 4, Example 15, Example 6 and Comparative Example 5), both eyes were administered simultaneously, the drugs were administered once in the morning and evening of each day, and all animals were given the same dosage. The application process ensured that the ointment was completely distributed in the cornea. One month after continuous administration, all animals' lens were observed with a slit lamp and scored, and the results were shown in Table 15. No eye discomfort symptoms occurred during the administration in all the animals tested.

TABLE 15
Pharmacodynamic evaluation of different groups on the treatment
of acquired cataract in vivo

| Administration | Animal | | One month after | |
|---|---|---|---|---|
| group | number | eye | treatment | Before treatment |
| Lanosterol | A | right | IV | V |
| eye ointment | | left | IV | VI |
| | B | right | V | V |
| | | left | V | IV |
| Inonotus | C | right | IV | V |
| obliquus | | left | IV | V |
| extract 1 eye | D | right | IV | V |
| ointment | | left | III | VI |
| Inotodiol eye | E | right | IV | V |
| ointment | | left | III | V |
| | F | right | IV | VI |
| | | left | III | V |
| low dose | G | right | V | V |
| lanosterol eye | | left | V | VI |
| ointment | H | right | V | V |
| | | left | V | IV |

Wherein, the score standard:

| Grade | Slit-lamp examination |
|---|---|
| I | Less than ⅕ torus, small blisters, fine brush-like |
| II | Less than ⅓ torus, cluster, slice, wedge |
| III | Less than ½ torus, ring, wedge, large slice |
| IV | Greater than or equal to ½ torus, block-like diffuse, |
| | radioactive pattern |
| V | Nuclear turbidity, less than or equal to ½ torus, block-like |
| | diffuse, radioactive pattern |
| VI | All turbidity |
| VII | lens atrophy, nuclear sinking |

As could be seen from Table 15, except lanosterol, the inonotus obliquus extract contained a large number of animal-safe ingredients that can be used to treat animal cataracts. These ingredients can be used to prepare drugs which were used to treat, prevent, stop and/or delay disease development of cataracts.

### Example 17

In Example 17, the rats were used as an animal model to investigate the eye drugs safety of inotodiol in Example 6, trametenolic acid in Example 7 and inonotus obliquus extract 1 in Example 15, and the blank eye ointment in Comparative Example 3 was used as a blank control simultaneously.

Eye Irritation Test Method:

Sixteen rabbits (half male, half female, weighing 2.3-3.0 kg) were divided into four groups, each group had two male and two female rabbits. The self was used as control. In the first group, the left eyes was given inotodiol eyes ointment in Example 6 and the right eye was given 0.9% sodium chloride injection. In the second group, the left eyes was given trametenolic acid eye ointment in Example 7 and the right eyes was given 0.9% sodium chloride injection. In the third group, the left eyes was given inonotus obliquus extract 1 eye ointment in Example 15 and the right eyes was given 0.9% sodium chloride injection. In the fourth group, the left eyes was given blank eye ointment in Comparative Example 3 and the right eyes was given 0.9% sodium chloride injection. The administered volume of eye ointment was about soybean size/eye, and the administered volume of 0.9% sodium chloride injection was 0.1 ml/eye.

Before the experiment, each rabbit's eyes were examined with sodium fluorescein, animals with eye irritation symptoms, corneal defects and conjunctival injury could not be used in the experiment, and the qualified rabbits were used in the experiment. The eyes were examined before the first administration and 1, 2, 4, 24, 48 and 72 hours after the last administration everyday. If no irritation symptom was observed at 72 h, the test could end. If mild irritation was observed, the observation period was extended to 7 days after the last administration. If moderate irritation was observed, the observation period was extended to 14 days after the last administration. If intensive irritation was observed, the observation period was extended to 21 days after the last administration. Administration times was three times a day, the interval of administration was at least 4 hours.

At the end of the experiment, the rabbits were sacrificed by carotid artery bloodletting after intraperitoneal injection of 20% ethyl carbamate solution, then the eyeballs were removed and fixed in the eyeball fixative solution for getting pathological material, dehydrating, embedding, staining and light microscopy examination.

Wherein, the scoring standard for eye irritation response observed during administration was shown in Table 16.

TABLE 16
The scoring standard for eye irritation response

| Eye irritation response | Score |
|---|---|
| Cornea: | |
| No turbidity | 0 |
| Dispersed or diffuse turbidity, the iris is clearly visible | 1 |
| Translucent area is easy to distinguish, the iris is unclear | 2 |
| The gray-white translucent area is appeared, the details of | 3 |
| iris were unclear, andthe pupil size is barely clear. | |
| The cornea is opaque and the iris is unrecognizable | 4 |
| Iris: | |
| normal | 0 |
| Wrinkles were obviously deepened, hyperaemia, swelling, | 1 |
| mild hyperaemia is around the cornea, and | |
| pupils still responds to light | |
| Hemorrhage/visual necrosis/no response to light | 2 |
| (or one of them) | |
| Conjunctiva: | |
| A hyperaemia (referring to the conjunctiva, bulbar | |
| conjunctiva area) | |
| Normal blood vessels | 0 |
| Blood vessel hyperaemia, bright red | 1 |
| Blood vessel hyperaemia, dark red, blood vessels are | 2 |
| difficult to distinguish | |
| Diffuse hyperaemia, purplish red | 3 |
| B edema | |
| No edema | 0 |
| Slight edema (including membranae nictitans) | 1 |
| Obvious edema with partial ectropion of eyelid | 2 |
| Edema to the eyelid nearly half close | 3 |
| Edema to eyelid more than half close | 4 |
| C secretion | |
| No secretion | 0 |
| A small amount of secretion | 1 |
| Secretions moisten or adhere eyelids and eyelashes | 2 |
| Secretions moisten or adher the entire eye area | 3 |
| Total score | 16 |

According to the requirements of Table 16, the irritation response scores of the cornea, iris and conjunctiva of each animal at each observation time point were added to obtain the total score, and the sum of the scores of a group was divided by the number of animals to obtain the final score. The degree of irritation was determined according to Table 17, and comprehensive judgment was made by combining with the results of histopathological examination.

TABLE 17
Eye irritation evaluation

| Score | evaluation |
|---|---|
| 0-3 | No irritation |
| 4-8 | Mild irritation |
| 9-12 | Moderate irritation |
| 13-16 | Intensive irritation |

Irritation Experiment Results:

After seven days of consecutive administration, the results of rabbit eye examination were as follows: in the first group, the second group, the third group and the fourth group, a small amount of secretion was seen in the left and right eyes of rabbits, the conjunctival hyperemia was bright red, and the iris was visible and there was mild hyperemia around the cornea. All eyes of the rabbits were normal at the end of 72 hours of administration, and no abnormal changes were observed, no cornea was stained yellow-green by fluorescein sodium examination. The irritation scores of the left and right eyes of rabbits in each group ranged from 0-3, and the results of eye irritation were judged as non-irritation.

The pathological examination results showed that after administrated with inotodiol eye ointment in Example 6, trametenolic acid eye ointment in Example 7, inonotus obliquus extract eye ointment in Example 15 and blank eye ointment in Comparative Example 3, the rabbit eye conjunctiva: no degeneration and no necrosis of epithelial cells in all parts, no expansion and no hyperaemia of interstitial blood vessels, and no inflammatory cell infiltration, etc.; cornea: no degeneration and no necrosis of corneal epithelial cells, no swelling and breakage of matrix collagen fibers, no inflammatory cell infiltration, no necrosis and no proliferation of corneal endothelial cells; iris: the structure of iris ciliary body tissue was clear, no interstitial hyperemia and no inflammatory cell infiltration; Harrington gland and lacrimal gland: glandular epithelial cells were normal, no interstitial hyperemia, no edema and no inflammatory cell infiltration.

In summary, the inotodiol eye ointment in Example 6, the trametenolic acid eye ointment in Example 7, the inonotus obliquus extract 1 eye ointment in Example 15 and the blank eye ointment in Comparative Example 3 had no irritation on rabbit conjunctiva, cornea, iris, Harrington gland and lacrimal gland, etc, suggesting eye drugs prepared by the inotodiol and trametenolic acid were safe.

### Example 18

The heatl stabilities of inotodiol and trametenolic acid were tested, and lanosterol was used as a reference at the same time. The evaluation method was carried out by self-control method, and each compound was placed at 4° C. and 80° C. for the same time, and HPLC (test concentration: 0.3 mg/ml, dissolved in methanol) was used to compare the percentage of changes in self-purity of inotodiol, trametenolic acid and lanosterol at 80° C. for different experimental time, the heat stability and druggability of three compounds with similar structures were determined by direct observation results of their appearance properties.

The initial experiment purities of the lanosterol, inotodiol and trametenolic acid stored at 4° C. were 96%, 94%, and 98% respectively. The contents of lanosterol, inotodiol and trametenolic acid were 73%, 88%, and 97% respectively after being placed at 80° C. for 1 day; the contents of lanosterol, inotodiol and trametenolic acid were 48%, 68%, and 96% respectively after being placed at 80° C. for 2 day; and the contents of lanosterol, inotodiol and trametenolic acid were 44%, 67%, and 96% respectively after being placed at 80° C. for 3 day.

After the lanosterol was stored at 80° C. for 1 day, the powder became hard and formed into a large piece. The color gradually turned yellow after continuing to place, one third of the lanosterol turned yellow after being placed for 1 week. The appearances of inotodiol and trametenolic acid did not change when they were placed at 80° C. for one week compared with 4° C. All lanosterol turned yellow after 2 weeks at 80° C., some of them showed crystallization, the agglomeration was serious, and the purity was only 32%. Inotodiol began to turn yellow after 2 weeks at 80° C. compared with 4° C. There was no significant change in appearance and purity of trametenolic acid after 2 weeks at 80° C. compared with 4° C. The purity of trametenolic acid remained above 93%.

The above results showed that the compound of formula I (represented by inotodiol and trametenolic acid) was more stable and had better druggability than lanosterol.

### Example 19

In the present Example 19, a series of chemical modification and derivative synthesis were carried out based on inotodiol and trametenolic acid, and some specific compounds obtained therein were identified and characterized.

A series of chemical modifications and derivative synthesis methods based on inotodiol are as follows:

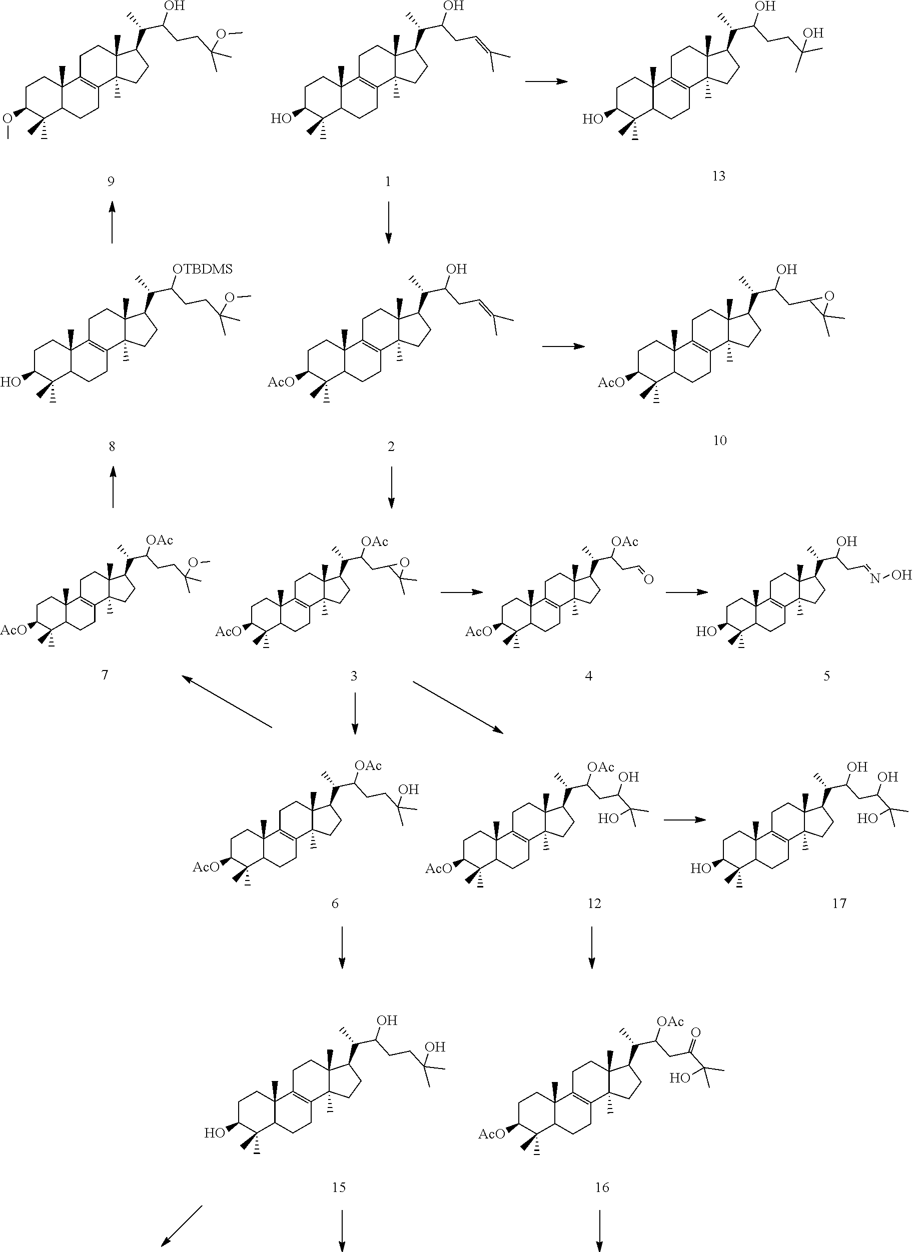

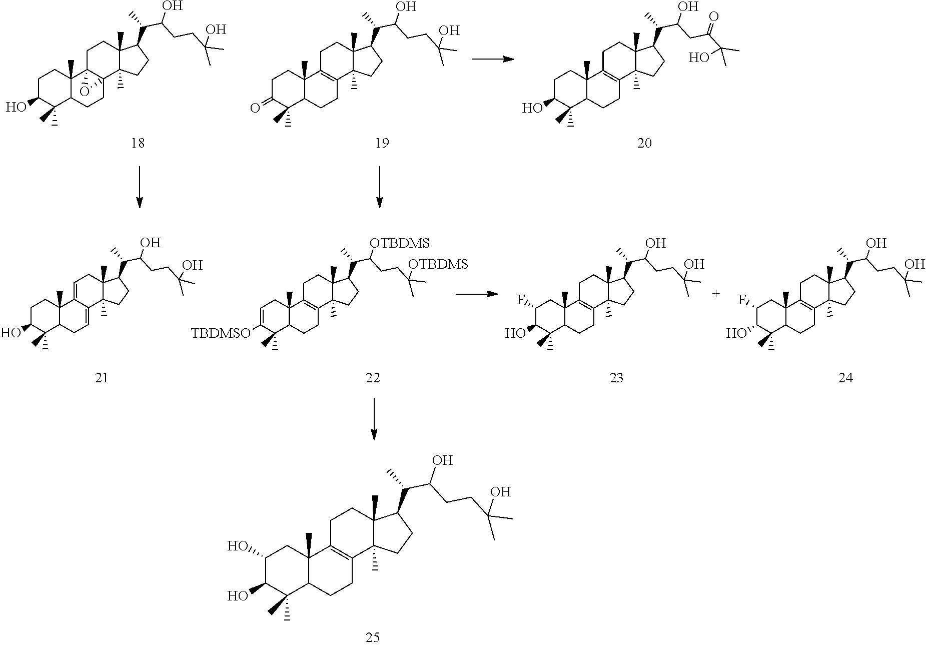

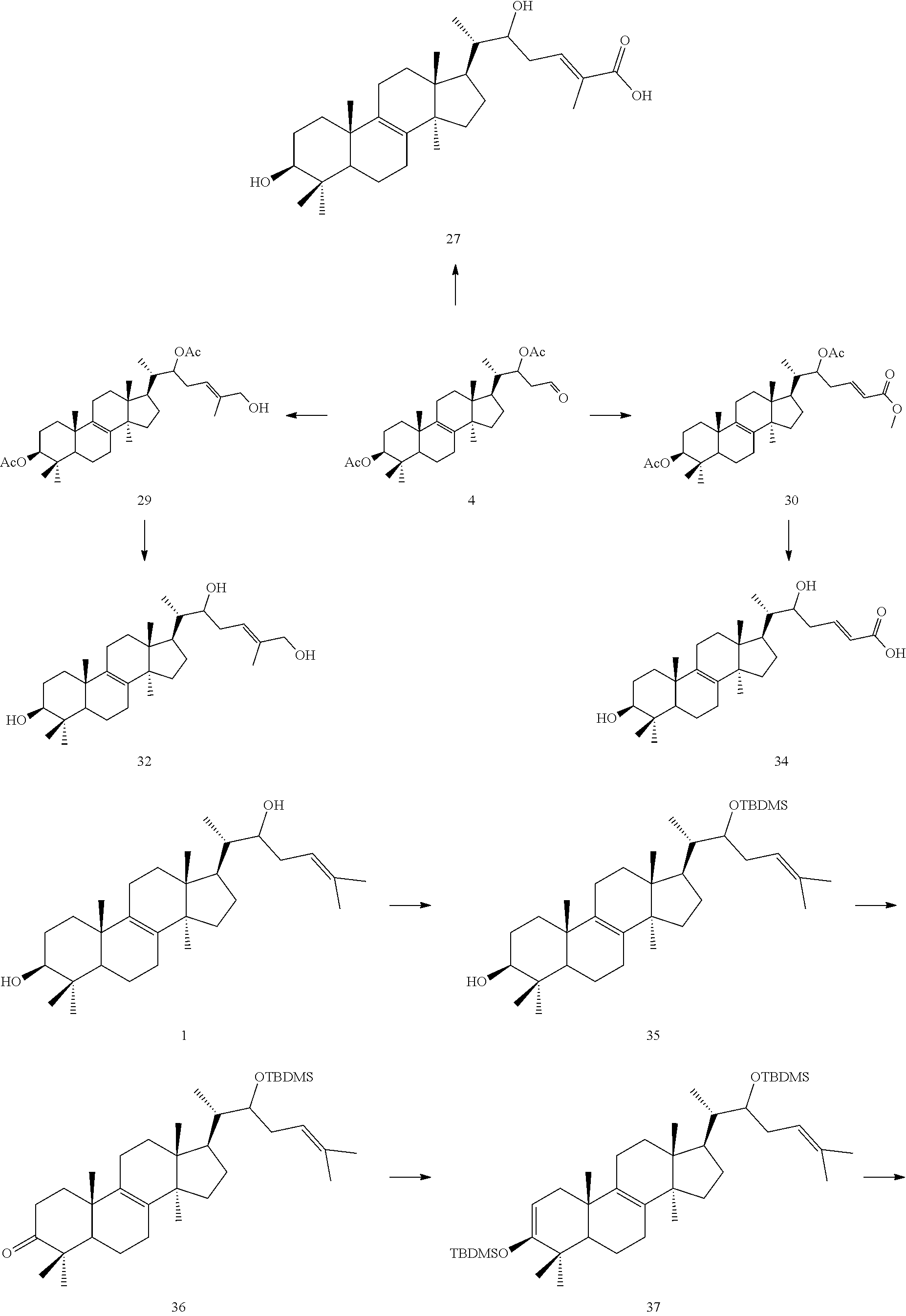

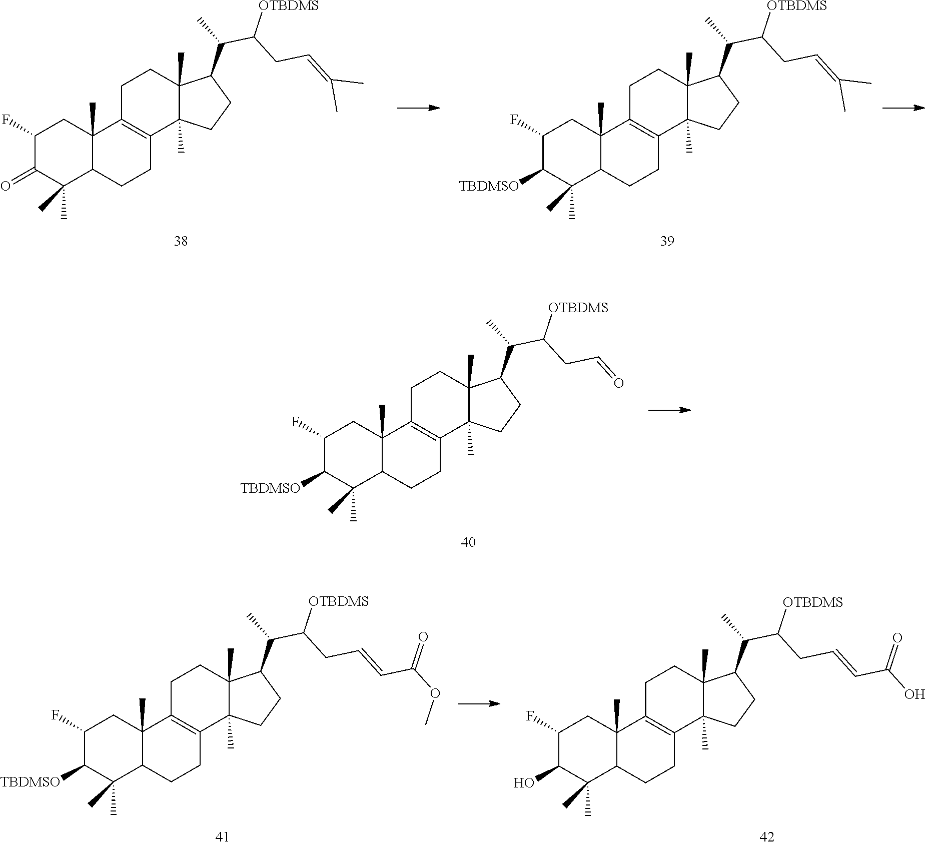

Wherein, part of the specific processes of a series of chemical modifications and derivative synthesis methods based on inotodiol were as follows.

Compound 1

Compound 1 was inotodiol.

Synthesis of Compound 2 and Compound 3

Compound 1 inotodiol (200 mg) was dissolved in dichloromethane (4 mL), acetic anhydride (180 mg) and pyridine (1 mL) were added, and the reaction was carried out by stirring at room temperature for 6 h. TLC showed the disappearance of the raw material, and the solvent was concentrated to obtain Compound 2 (240 mg).

Compound 2 (200 mg) was dissolved in dichloromethane (4 mL), and m-CPBA (m-chloroperoxybenzoic acid) (85%, 85 mg) and sodium bicarbonate (42 mg) were added to the above solution in three batches at intervals of 2 h in ice bath, then washed once with sodium bicarbonate aqueous solution, concentrated and subjected to column chromatography to afford compound 3 (154 mg).

Synthesis of Compound 4

Sodium periodate (87 mg) was added to 10 mL of diethyl ether, and compound 3 (200 mg) was added under stirring, and reaction was carried out at room temperature for 30 min. Compound 4 (140 mg) was obtained by column chromatography after washing with water, stratification, and concentration of organic phase.

Synthesis of Compound 5

Compound 4 (200 mg) was dissolved in 10 mL of ethanol, hydroxylamine hydrochloride (31 mg), and potassium carbonate (165 mg) were added, and reaction was carried out under stirring at 50° C. for 6 h. The mixture was cooled, filtered, concentrated and subjected to column chromatography to afford compound 5 (147 mg).

1H NMR (CD3Cl, 400 MHz): δ (ppm) 3.70 (1H, m), 3.24 (1H, dd, J=11.6 Hz, J=4.4 Hz), 0.73, 0.82, 0.88, 0.99, 1.01 (3H each, s), 0.97 (3H, d, J=6.8); 13C NMR (CDCl₃, 100 MHz): δ (ppm) 153.2, 134.5, 134.2, 79.0, 73.4, 50.4, 49.4, 47.2, 44.8, 41.6, 38.9, 37.0, 35.6, 30.9, 29.1, 28.0, 27.8, 27.2, 26.5, 26.0, 24.3, 21.0, 19.2, 18.2, 18.0, 15.7, 15.4, 12.6.

Synthesis of Compound 6

Sodium bis(2-methoxyethoxy)aluminiumhydride solution (70% toluene solution, 0.1 mL) was added to tetrahydrofuran (10 mL), and compound 3 (200 mg) was added, and the reaction was carried out at −10° C. for 5 h. Then the mixture was poured into dilute hydrochloric acid, and extracted with ethyl acetate, dried over sodium sulfate. Compound 6 (120 mg) was obtained by column chromatography after concentration.

Synthesis of Compound 7

Compound 6 (200 mg) was dissolved in 10 mL of tetrahydrofuran, sodium hydrogen (16 mg) and methyl iodide (58 mg) were added, and the reaction was carried out under stirring at room temperature for 12 h. After the reaction, the mixture was concentrated to remove solvent, then dissolved in dichloromethane, washed with water, concentrated and subjected to column chromatography to afford compound 7 (85 mg).

Synthesis of Compound 9

Compound 7 (300 mg) was dissolved in methanol (6 mL), potassium carbonate (150 mg) was added, the reaction was carried out at 50° C. for 6 h. The mixture was concentrated after filtration, followed by adding dichloromethane (6 mL), pyridine (1 mL), and TBDMSCl (80 mg), then the reaction was carried out at room temperature until the substrate disappears. Methyl iodide (80 mg) was added, and the reaction was carried out at room temperature for 12 h, then the mixture was washed with water. After the solvent was removed, tetrahydrofuran (6 mL) and tetrabutylammonium fluoride (280 mg) were added, the reaction was carried out at room temperature for 5 h. Solvents were removed by concentration. The mixture was dissolved by adding dichloromethane, washed with water and concentrated. Then compound 9 (90 mg) was obtained by column chromatography.

1H NMR (CD3Cl, 400 MHz): δ(ppm) 3.37 (3H, s), 3.16 (3H, s) 0.68 (3H, s); 13C NMR (CDCl3, 100 MHz): δ(ppm) 134.7, 134.5, 88.8, 73.4, 73.3, 57.7, 51.4, 50.7, 49.4, 47.2, 44.8, 41.6, 39.0, 37.2, 37.0, 36.6, 35.7, 30.9, 29.5, 28.0, 27.8, 26.8, 26.5, 24.4, 22.8, 21.2, 19.2, 18.2, 18.0, 16.3, 12.6.

Synthesis of Compound 10

Compound 2 (200 mg) was dissolved in ethanol (4 mL), sodium hydroxide (15 mg) was added, and the reaction was carried out at room temperature for 6 h and then the solvent was removed by concentration. The mixture was subjected to column chromatography and dissolved in dichloromethane (4 mL). Then m-CPBA (m-chloroperoxybenzoic acid) (85%, 50 mg) and sodium bicarbonate (20 mg) were added to the above solution in three batches at intervals of 2 h in ice bath, then washed once with sodium bicarbonate solution, and Compound 10 (100 mg) were obtained by column chromatography after concentration.

¹H NMR (CD₃Cl, 400 MHz): δ(ppm) 4.50 (1H, m), 3.74 (1H, m) 3.24 (1H, dd) 2.68 (1H, t, J=6.4 Hz), 0.69 (3H, s); ¹³C NMR (CDCl₃, 100 MHz): δ(ppm) 171.2, 134.5, 134.2, 81.0, 72.2, 63.5, 58.4, 52.0, 50.4, 49.9, 44.8, 41.6, 37.9, 37.0, 35.5, 30.9, 29.1, 26.0, 27.8, 25.8, 25.1, 25.0, 24.3, 23.0, 21.0, 19.2, 18.9, 18.8, 18.7, 18.4, 12.4.

Synthesis of Compound 12

Compound 3 (200 mg) was dissolved in isopropanol (5 mL), water (2 mL) and hypophosphite (0.5 mL, 50% aqueous solution) were added, and the mixture was refluxed for 3 hours and then cooled. 20 mL water was added. A white solid compound 12 was obtained by filtration and drying.

Synthesis of Compound 13

Compound 1 (200 mg) was dissolved in tetrahydrofuran-water mixture (1:1, 10 mL), NBS (80 mg) was added, and the reaction was carried out at room temperature for 2 h, tetrahydrofuran was removed by concentration, the aqueous phase was extracted by adding dichloromethane. Compound 13 (190 mg) was obtained by column chromatography after solvent removal by concentration.

1H NMR (CD₃Cl, 400 MHz): δ(ppm) 4.0-3.9 (m, 1H), 3.2 (1H, m), 0.69 (3H, s); 13C NMR (CDCl3, 100 MHz): δ(ppm) 134.5, 134.2, 79, 74, 70.7, 63.8, 50.4, 49.4, 47.2, 44.8, 41.8, 38.9, 37, 35.6, 35.2, 30.9, 29.1, 29.1, 27.8, 27.8, 26.5, 26, 26, 24.3, 21, 19.2, 15.4, 15.4, 12.7, 12.6.

Synthesis of Compound 15

Compound 6 (200 mg) was dissolved in methanol (4 mL), potassium carbonate (100 mg) was added, the reaction was carried out at 50° C. for 6 h, and the mixture was concentrated after filtration. Compounds 15 (145 mg) were obtained by column chromatography.

¹H NMR (CD₃Cl, 400 MHz): δ(ppm) 3.7 (1H, m), 3.25 (1H, m) 0.69 (3H, s); ¹³C NMR (CDCl₃, 100 MHz): δ(ppm) 134.5, 134.2, 79.0, 73.4, 70.3, 50.4, 49.4, 47.2, 44.8, 41.6, 40.0, 38.9, 37.0, 35.6, 30.9, 29.5, 29.1, 28.0, 27.8, 26.8, 26.5, 26.0, 24.3, 21.0, 19.2, 18.2, 18.0, 15.7, 15.4, 12.6.

Synthesis of Compound 17

Compound 12 (200 mg) was dissolved in ethanol (10 mL), potassium carbonate (100 mg) was added, and the reaction was carried out at room temperature for 16 h, then the mixture was concentrated after filtration. Compound 17 (130 mg) were obtained by column chromatography.

¹H NMR (CD₃Cl, 400 MHz): δ(ppm) 4.5-4.4 (m, 1H), 3.35 (1H, m), 0.68 (3H, s); ¹³C NMR (CDCl₃, 100 MHz): δ(ppm) 134.5, 134.2, 79, 75.4, 74.2, 68.3, 50.4, 49.4, 47.2, 44.8, 42, 38.9, 37, 35.6, 34.3, 30.9, 29.1, 29.1, 27.8, 27.8, 26.5, 25.7, 25.7, 24.3, 21, 19.2, 15.4, 15.4, 12.7, 12.6.

Synthesis of Compound 19

Compound 15 (200 mg) was dissolved in dichloromethane (6 mL). Pyridine (1 mL) and TBDMSCl (65 mg) were added, the reaction was carried out at room temperature until the substrates disappear. After the mixture was washed with water, Dess-Martin periodinane (170 mg) was added, then the reaction was carried out at room temperature for 6 h, and the mixture was washed with sodium bicarbonate aqueous solution, and concentrated to remove solvent. Then tetrahydrofuran (6 mL), and tetrabutylammonium fluoride (170 mg) were added, the reaction was carried out at room temperature for 5 h. Dichloromethane was added after concentration, then the mixture was washed with water and concentrated. Then Compound 19 (93 mg) was obtained by column chromatography.

Synthesis of Compound 20

Compound 12 (200 mg) was dissolved in dichloromethane (5 mL), then Dess-Martin periodinane (170 mg) was added, the reaction was carried out at room temperature for 6 h, the mixture was washed with sodium bicarbonate aqueous solution and concentrated to remove solvent, 10 mL ethanol and potassium carbonate (100 mg) were added, the reaction was carried out at room temperature for 16 h, then the mixture was concentrated after filtration. Compound 20 (102 mg) was obtained by column chromatography.

¹H NMR (CD₃Cl, 400 MHz): δ(ppm) 3.86 (1H, m), 3.23 (1H, d), 2.62-2.51 (2H, m), 0.69 (3H, s).

Synthesis of Compound 21

Compound 15 (400 mg) was dissolved in dichloromethane (20 mL), and m-CPBA (m-chloroperoxybenzoic acid) (85%, 180 mg) and sodium bicarbonate (60 mg) were added to the above solution in three batches at intervals of 2 h in ice bath, and the reaction was carried out at room temperature overnight, the mixture was washed once with sodium bicarbonate aqueous solution, the solvent was removed by concentration and tetrahydrofuran (10 mL) was added, 100 uL of 40% hydrofluoric acid solution was added, and the reaction was carried out at room temperature for 4 days. Sodium bicarbonate aqueous solution was added and the mixture was extracted with dichloromethane. Compound 21 (260 mg) was obtained by column chromatography after the organic phase was concentrated.

¹H NMR (CD₃Cl, 400 MHz): δ(ppm) 5.49 (1H, m), 5.33 (1H, m), 3.24 (1H, m), 0.58 (3H, s); ¹³C NMR (CDCl₃, 100 MHz): δ(ppm) 146, 142.8, 120.3, 116.5, 79.1, 73.4, 70.3, 51.2, 50.5, 49.2, 44.5, 41.6, 41.6, 38.8, 38, 37.5, 36.8, 31.6, 29.5, 29.1, 27.9, 26, 25.7, 25.7, 23.1, 23.1, 22.9, 21.3, 15.9, 12.6.

Synthesis of Compound 22

Compound 19 (200 mg) was dissolved in dichloromethane (6 mL), triethylamine (1 mL) and TBSOTf (340 mg) were added and the reaction was carried out at room temperature for 2 h. The mixture was concentrated to remove solvent after being washed with water. Then compound 22 (302 mg) was obtained by column chromatography.

Synthesis of Compound 23 and Compound 24

Compound 22 (200 mg) was dissolved in DMF (6 mL) and 1-chloromethyl-4-fluoro-1,4-diazide-bicyclo-2.2.2 octane bis(tetrafluoroborate) (selectfluor, 88 mg) 50 was added, the reaction was carried out at room temperature for 1 h. Dichloromethane was added, the solvent was removed under reduced pressure after washed with sodium bicarbonate aqueous solution, ethanol (6 mL) and sodium borohydride (10 mg) were added, then the reaction was carried out at room temperature for 1 h. Dilute hydrochloric acid was added to the above system, and concentrated to remove the solvent. After extraction with ethyl acetate and concentration, white solids were obtained by column chromatography. Tetrahydrofuran (6 mL), and tetrabutyl ammonium fluoride (130 mg) were added, and the reaction was carried out at room temperature for 5 h. Dichloromethane was added after concentration. After washed with water and concentration, compound 23 (43) and compound 24 (8 mg) were separated by preparative liquid chromatography.

Compound 23: 1H NMR (CD3Cl, 400 MHz): δ(ppm) 5.01-4.86 (1H, m), 3.68 (1H, d), 0.67 (3H, s); 13C NMR (CDCl3, 100 MHz): δ(ppm) 134.7, 133.9, 91.4, 76.7, 73.0, 71.1, 50.5, 49.9, 47.2, 46.3, 42.5, 41.6, 35.8, 35.6, 31.0, 30.9, 29.7, 29.5, 29.4, 28.4, 28.2, 26.2, 25.4, 24.3, 21.3, 21.2, 20.3, 18.8, 15.4, 12.6.

Compound 24: 1H NMR (CD3Cl, 400 MHz): δ(ppm) 4.64-4.50 (1H, m), 3.28 (1H, dd), 0.68 (3H, s); 13C NMR (CDCl3, 100 MHz): δ(ppm) 134.9, 133.5, 93.4, 81.1, 73.0, 71.1, 50.4, 49.8, 47.2, 46.3, 42.5, 41.6, 36.7, 36.5, 30.8, 30.7, 29.7, 29.3, 29.2, 28.4, 28.2, 26.2, 25.4, 24.3, 21.3, 21.1, 18.7, 18.0, 15.4, 12.6.

Synthesis of Compound 25

Compound 22 (400 mg) was dissolved in dichloromethane (10 mL), and m-CPBA (m-chloroperoxybenzoic acid) (85%, 100 mg) and sodium bicarbonate (40 mg) were added to the above solution in three batches at intervals of 2 h in ice bath, then washed once with sodium bicarbonate aqueous solution, tetrahydrofuran (10 mL), and tetrabutyl ammonium fluoride (400 mg) were added after concentration, the reaction was carried out at room temperature for 5 h, and dichloromethane was added after concentration, and after washed with water and concentration, compound 25 (144 mg) was obtained by column chromatography.

¹H NMR (CD₃Cl, 400 MHz): δ(ppm) 3.65 (1H, m), 2.93 (1H, d), 0.64 (3H, s); ¹³C NMR (CDCl₃, 100 MHz): δ(ppm) 135.48, 135.14, 83.97, 73.4, 70.3, 69.6, 51.49, 50.61, 47.2, 45.38, 44.98, 41.6, 41.6, 40.02, 38.91, 31.88, 31.57, 29.5, 29.1, 28.95, 28.91, 27.18, 26, 24.55, 21.97, 21.84, 19.12, 19.09, 16.18, 12.6.

Synthesis of Compound 27

Compound 4 (200 mg) was dissolved in DMF (5 mL), trimethyl phosphonopropionate (78 mg), and potassium carbonate (60 mg) were added and the reaction was carried out at room temperature for 6 h. The mixture was poured into water and extracted with dichloromethane, after the solvent was removed by concentration, tetrahydrofuran (5 mL) and lithium hydroxide aqueous solution (10%, 0.3 mL) were added, and the reaction was carried out at room temperature for 6 h. Tetrahydrofuran was removed by concentration, water (5 mL) was added, and pH was adjusted to pH 2.5 with dilute hydrochloric acid. The mixture was filtered and dried to afford compound 27 (132 mg).

¹H NMR (CD₃Cl, 400 MHz): δ(ppm) 6.8 (1H, m), 3.77 (1H, m) 3.16 (1H, m) 0.66 (3H, s); ¹³C NMR (CDCl₃, 100 MHz): δ(ppm) 170.5, 140.1, 134.5, 134.2, 128.8, 78.6, 72.3, 50.4, 49.8, 46.6, 44.3, 41.1, 38.7, 36.9, 35.6, 34.8, 31, 30.7, 27.7, 27.5, 27.3, 26.4, 24, 20.9, 18.9, 18.2, 15.5, 15.2, 12.2, 11.5.

Synthesis of Compound 32

Preparation of Wittig reagent: 2-bromo-n-propanol (100 mg) was dissolved in dichloromethane (5 mL), triphenylphosphine (189 mg) was added, refluxed for 16 h, and filtered to obtain wittig reagent.

DMF (5 mL) was added to the above wittig reagent (160 mg), sodium hydrogen (16 mg) was added and stirred at room temperature for 2 h, and then compound 4 (200 mg) was added, the reaction was carried out at room temperature for 6 h, the mixture was poured into water and filtered, and the filtered cake was dissolved in tetrahydrofuran (5 mL), and lithium hydroxide aqueous solution (10%, 0.2 mL), the reaction was carried out at room temperature for 6 h, the tetrahydrofuran was removed by concentration, water (5 mL) was added, and the mixture was extracted with dichloromethane and concentrated. Then Compound 32 (120 mg) were obtained by column chromatography.

¹H NMR (CD₃Cl, 400 MHz): δ(ppm) 5.85 (1H, m) 4.04 (2H, s) 3.7 (1H, m), 3.25 (1H, dd) 0.63 (3H, s); ¹³C NMR (CDCl₃, 100 MHz): δ(ppm) 137.6, 134.5, 134.2, 121.4, 79, 73.4, 68.7, 50.4, 49.4, 47.2, 44.8, 41.6, 38.9, 37, 35.6, 33.9, 30.9, 30.9, 29.1, 28, 27.8, 26.5, 24.3, 21, 19.2, 18.2, 15.7, 15.4, 12.9, 11.8.

Synthesis of Compound 34

Compound 4 (200 mg) was dissolved in DMF (5 mL), trimethyl phosphonoacetate (78 mg), and potassium carbonate (60 mg) were added and the reaction was carried out at room temperature for 6 h. The mixture was poured into water and extracted with dichloromethane, after the solvent was removed by concentration, tetrahydrofuran (5 mL) and lithium hydroxide aqueous solution (10%, 0.3 mL) were added, the reaction was carried out at room temperature for 6 h. Tetrahydrofuran was removed by concentration, water (5 mL) was added, and pH was adjusted to 2.5 with dilute hydrochloric acid. The mixture was filtered and dried to afford compound 34 (138 mg).

¹H NMR (CD₃Cl, 400 MHz): δ(ppm) 7.02 (1H, dt), 6.4-6.7 (1H, m), 3.8 (1H, m) 3.18 (1H, m) 0.67 (3H, s); ¹³C NMR (CDCl₃, 100 MHz): δ(ppm) 170.5, 145.1, 134.5, 134.2, 121.8, 78.6, 72.3, 50.4, 49.8, 46.6, 44.3, 41.1, 38.7, 36.9, 35.6, 31.8, 31, 30.7, 27.7, 27.5, 27.3, 26.4, 24, 20.9, 18.9, 18.2, 15.5, 15.2, 11.5.

Synthesis of Compound 42

Compound 1 (600 mg) was dissolved in dichloromethane (12 mL), pyridine (1 mL), and TBDMSCl (203 mg) were added, and the reaction was carried out at room temperature for 6 h. The mixture were washed with water and dried over sodium sulfate. Dess-Martin Oxidizer (170 mg) was added, and the reaction was carried out at room temperature for 6 h, the mixture was washed with sodium bicarbonate aqueous solution and dried over sodium sulfate, then triethylamine (1 mL) and TBSOTf (358 mg) were added, and the reaction was carried out at room temperature for 2 h, the mixture was washed with water and concentrated to remove solvent, then DMF (20 mL) and 1-chloromethyl-4-fluoro-1,4-diazotized bicyclic 2.2.2 octane bis(tetrafluoroborate) (selectfluor, 336 mg) were added, the reaction was carried out at room temperature for 1 h, dichloromethane was added, the mixture was washed with sodium bicarbonate aqueous solution and concentrated under reduced pressure to remove solvent, and ethanol (20 mL) and sodium borohydride (30 mg) were added, and the reaction was carried out at room temperature for 1 h, the pH was adjusted to 4.5 with dilute hydrochloric acid, the solvent was removed by concentration, the mixture was extracted with ethyl acetate, dried over sodium sulfate, and concentrated, then dichloromethane (20 mL), triethylamine (1 mL), and TBSOTf (358 mg) were added, the reaction was carried out at room temperature for 2 h, the mixture was washed with water and concentrated to remove solvent. Then Compound 39 (116 mg) was obtained by column chromatography. Compound 39 (100 mg) was dissolved in dichloromethane (4 mL), and m-CPBA (m-chloroperoxybenzoic acid) (85%, 30 mg) and sodium bicarbonate (20 mg) were added to the above solution in three batches at intervals of 2 h in ice bath, the mixture was washed once with sodium bicarbonate aqueous solution, and concentrated to remove solvent, and added into sodium periodate (31 mg)-diethyl ether (5 mL) solution, the reaction was carried out at room temperature for 30 min. The mixture was washed with water and separated, then the organic phase was concentrated, DMF (5 mL), trimethyl phosphonylacetate (26 mg) and potassium carbonate (20 mg) were added and heated to 50° C. for 2 h. The mixture was poured into water and extracted with ethyl acetate, tetrahydrofuran (6 mL) and lithium hydroxide aqueous solution (10%, 0.1 mL) were added after concentration, the reaction was carried out at room temperature for 16 h, the water was added, and the pH was adjusted to 2.5, then tetrahydrofuran was removed by concentration. The mixture was extracted with ethyl acetate and concentrated, then Compound 42 (35 mg) was obtained by liquid chromatography.

1H NMR (CD3Cl, 400 MHz): δ(ppm) 7.02 (1H, dt), 6.4-6.7 (1H, m), 5.01-4.86 (1H, m), 3.8 (1H, m), 3.7 (1H, d), 0.67 (3H, s); 13C NMR (CDCl3, 100 MHz): δ(ppm) 170.5, 145.1, 134.5, 134.2, 121.8, 91.4, 76.7, 72.3, 50.4, 49.8, 46.6, 44.3, 41.1, 38.7, 36.9, 35.6, 31.8, 31, 30.7, 27.7, 27.5, 26.4, 24, 20.9, 18.9, 18.2, 15.5, 15.2, 11.5.

A series of chemical modifications and derivative synthesis methods based on trametenolic acid are as follows.

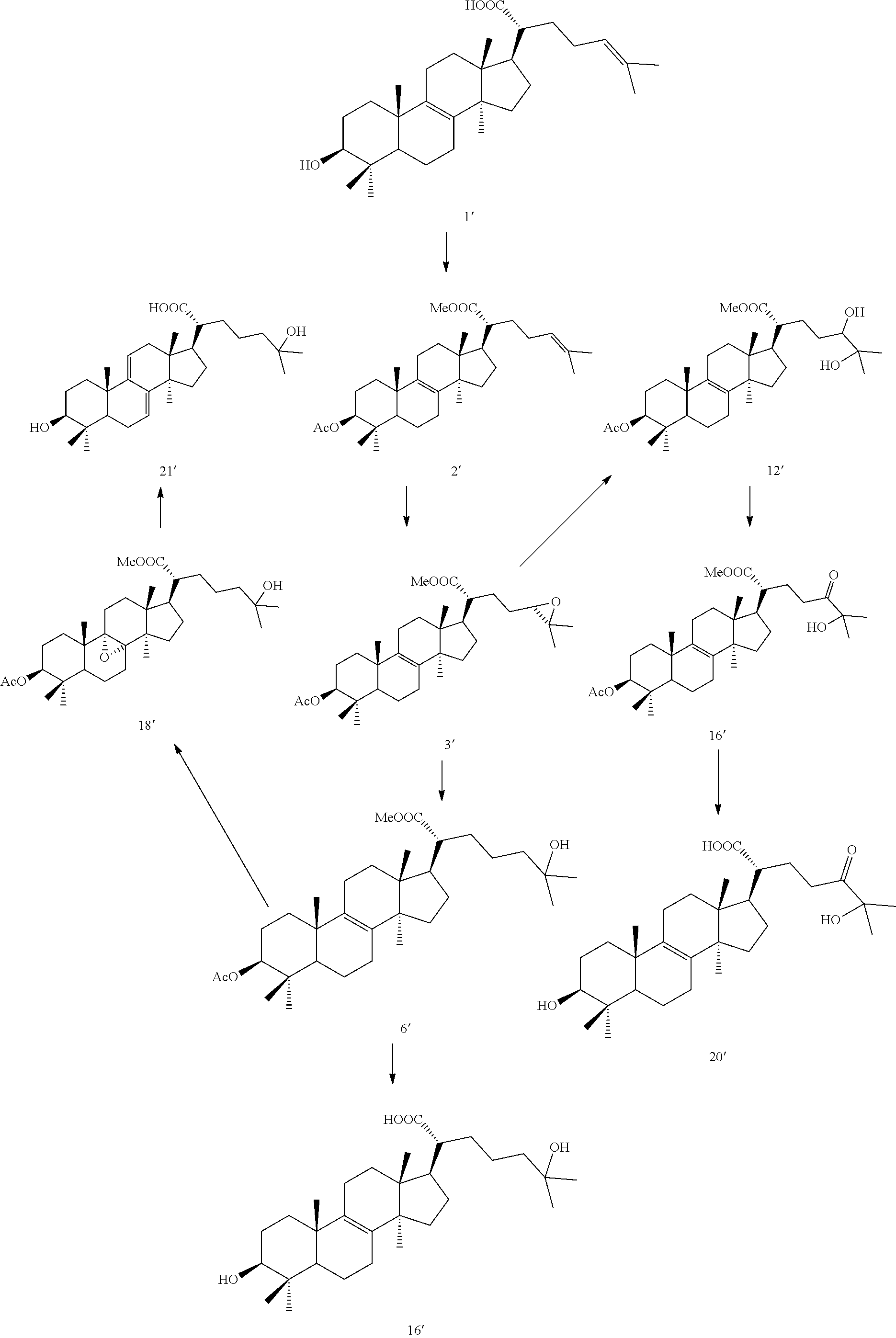

Wherein, part of the specific processes of a series of chemical modifications and derivative synthesis methods based on trametenolic acid are as follows.

Compound 1′

Compound 1′ was trametenolic acid.

Synthesis of Compound 3′

Compound 1 trametenolic acid (200 mg, extracted from inonotus obliquus) was dissolved in methanol (4 mL), and thionyl chloride (52 mg) was added at room temperature. The reaction was carried out under stirring at room temperature for 1 h, and the solvent was removed by concentration, dichloromethane (4 mL), and acetic anhydride (45 mg) were added, the reaction was carried out at room temperature for 6 h. m-CPBA (m-chloroperoxybenzoic acid) (85%, 85 mg) and sodium bicarbonate (80 mg) were added to the above solution in three batches at intervals of 2 h in ice bath, and the mixture was washed once with sodium bicarbonate aqueous solution and concentrated. Then Compounds 3′ (162 mg) were obtained by column chromatography.

Synthesis of Compound 6′

Sodium bis(2-methoxyethoxy)aluminiumhydride solution (70% toluene solution, 0.1 mL) was added to tetrahydrofuran (10 mL), and compound 3′ (200 mg) was added, and the reaction was carried out at −20° C. for 5 h, the mixture was poured into dilute hydrochloric acid, and extracted with ethyl acetate, dried over sodium sulfate and concentrated. Then Compound 6′ (106 mg) was obtained by column chromatography.

Synthesis of Compound 15′

Compound 6′ (200 mg) was dissolved in tetrahydrofuran (4 mL), lithium hydroxide (10%, 0.2 mL) aqueous solution was added and the reaction was carried out at room temperature for 6 h. After the mixture was concentrated to remove tetrahydrofuran, the pH was adjusted to 2.5, the mixture was was extracted with ethyl acetate and concentrated. Then Compounds 15′(140 mg) were obtained by column chromatography.

¹H NMR (DMSO, 400 MHz): δ(ppm) 3.04 (1H, m) 0.68 (3H, s); ¹³C NMR (DMSO, 100 MHz): δ(ppm) 177.5, 134.8, 133.9, 77.2, 71.4, 50.5, 49.5, 47.9, 47, 44.7, 44.3, 39, 37, 35.7, 32.8, 30.7, 29.5, 29.4, 28.8, 28.6, 28, 27.8, 26.5, 24.5, 21.1, 20.6, 19.4, 18.4, 16.3, 16.1.

Synthesis of Compound 12′

Compound 3′ (200 mg) was dissolved in isopropanol (5 mL), water (2 mL) and hypophosphorous acid (0.5 mL, 50% aqueous solution) were added, and the mixture was refluxed for 3 hours and then cooled. 20 mL water was added to the mixture which was then filtrated and dried to obtain a white solid compound 12′.

Synthesis of Compound 20′

Compound 12′(200 mg) was dissolved in dichloromethane (5 mL), then Dess-Martin periodinane (170 mg) was added, the reaction was carried out at room temperature for 6 h, the mixture was washed with sodium bicarbonate and concentrated, 10 mL ethanol and potassium carbonate (100 mg) were added, the reaction was carried out at room temperature for 16 h, then the water was added. After the pH was adjusted to 2.5, the mixture was extract with ethyl acetate, and concentrated. Then Compound 20′ (102 mg) was obtained by column chromatography.

¹H NMR (DMSO, 400 MHz): δ(ppm) 3.1 (1H, dd), 2.17 (1H, m) 0.72 (3H, s); ¹³C NMR (DMSO, 100 MHz): δ(ppm) 215.4, 177.3, 134.8, 133.9, 79, 77.2, 50.5, 49.5, 47.9, 47.1, 44.3, 39, 37, 35.7, 32.8, 31, 30.7, 28.8, 28.7, 28.6, 26.7, 26.67, 26.5, 24.8, 21.3, 21.1, 19.4, 18.4, 16.3, 16.2.

Synthesis of Compound 21′

Compound 6′ (400 mg) was dissolved in dichloromethane (20 mL), and m-CPBA (m-chloroperoxybenzoic acid) (85%, 129 mg) and sodium bicarbonate (50 mg) were added to the above solution in three batches at intervals of 2 h in ice bath, and the reaction was carried out at room temperature overnight, the mixture was washed once with sodium bicarbonate aqueous solution, the solvent was removed by concentration and tetrahydrofuran (10 mL) was added, 100 uL of 40% hydrofluoric acid solution was added, and the reaction was carried out at room temperature for 4 days, sodium bicarbonate aqueous solution was added and the mixture was extracted with dichloromethane, after the organ phase was concentrated, tetrahydrofuran (10 mL), lithium hydroxide aqueous solution (10%, 0.4 mL) were added, the reaction was carried out at room temperature for 16 h, the solvent was removed by concentration, after the water was added, the pH was adjusted to 2.5, the mixture was extracted with ethyl acetate and dried over sodium sulfate and concentrated, then compound 21′ (106 mg) was obtained by column chromatography.

1H NMR (DMSO, 400 MHz): δ(ppm) 5.62 (1H, d), 5.36 (1H, d), 3.48 (1H, m), 1.01 (3H, s); 13C NMR (DMSO, 100 MHz): δ(ppm) 177.5, 146, 142.8, 121, 116.5, 79.1, 71.4, 50.5, 49.2, 48.1, 47, 44.7, 44.3, 38.8, 37.5, 36.4, 36, 31.6, 29.5, 29.4, 28.8, 28.1, 27.8, 27.3, 25.9, 23.3, 22.9, 20.6, 16.6, 16.3.

### Example 20

In Example 20, dried inonotus obliquus was used as a raw material, and after the raw material was pulverizated, and subjected to a series of separation operations of chemical components such as purification, extraction, and silica gel column chromatography to obtain 10 specific compounds.

TABLE 18
Chemical structure identification and extraction methods of Inonotus obliquus extract

| No. | Structural formula | ¹H NMR & ¹³C NMR | extraction methods |
|---|---|---|---|
| 1″ | 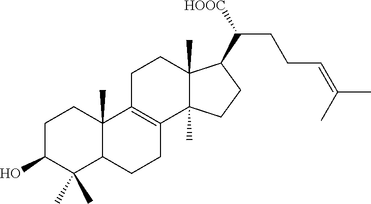 | 1H NMR (C5D5N, 400 MHz): 0.989 (3H, s, H3-18), 0.955 (3H, s, H3-30), 1.050 (3H, s, H3-28), 1.141 (3H, s, H3-19), 1.168 (3H, s, H3-29), 1.596 (3H, s, H3-25), 1.671 (3H, s, H3-27), 3.420 (1H, dd, J = 7.2 Hz, H-3), 4.430 (1H,d, J = 4.8 Hz, OH), 5.302 (1H, t, J = 7.2 Hz, H-24). 13C NMR (C5D5N, 100 MHz): 37.71 (C-1), 30.99 (C-2), 79.6 (C-3), 41.13 (C-4), 52.49 (C-5), 20.31 (C-6), 30.23 (C-7), 136.76 (C-8), 135.88 (C-9), 38.98 (C-10), 22.58 (C-11), | 10 kg of the crude powder of the sporophore of Inonotus obliquus was refluxed with ethanol, and the extract was distilled off under reduced pressure to remove solvent to obtain 200 g of ethanol extract. The ethanol extract was suspended with water, the mixture was extracted with petroleum ether and ethyl acetate, and the extraction solution were concentrated to afford a |
| | | 32.47 (C-12), 46.49 (C-13), | petroleum ether extract (12 g) |
| | | 51.44 (C-14), 34.91 (C-15), 29.09 | and ethyl acetate extract (8 g). |
| | | (C-16), 50.63 (C-17), 17.98 (C-18), | The ethyl acetate extract was |
| | | 21.01 (C-19), 49.33 (C-20), 180.24 | separated by column |
| | | (C-21), 28.41 (C-22), 28.34 (C-23), | chromatography with |
| | | 126.48 (C-24), 133.31 (C-25), | chloroform-methanol (100: |
| | | 27.39 (C-26), 19.33 (C-27), 30.3 | 1-10:1) gradient elution, the |
| | | (C-28), 17.94 (C-29), 26.1 (C-30). | mixture III was obtained by |
| | | | elution with |
| | | | chloroform-methanol (50:1). The |
| | | | mixture III was subjected to |
| | | | silica gel column |
| | | | chromatography with diethyl |
| | | | ether-ethyl acetate (20:1 to 1:2) |
| | | | gradient elution, the mixture IV |
| | | | was obtained by elution with |
| | | | diethyl ether-ethyl acetate (3:1). |
| | | | The mixture IV was subjected to |
| | | | silica gel column |
| | | | chromatography with diethyl |
| | | | ether-ethyl acetate (5:1-1:2), |
| | | | compound 1″was obtained by |
| | | | elution with diethyl ether-ethyl |
| | | | acetate (2:1) and identified as |
| | | | trametenolic acid. Among them, |
| | | | 50 mL of the eluate was collected |
| | | | each time, and the same fraction |
| | | | was combined according to the |
| | | | TLC detection result, and the |
| | | | single component was |
| | | | recrystallized. |
| 2″ | 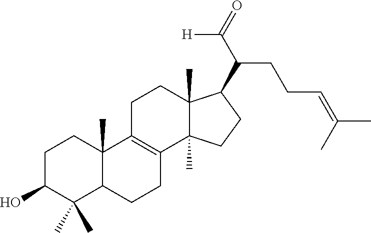 | 1H NMR (CDCl3, 500 MHz) δ: 0.68 (3H, s), 0.80 (3H, s), 0.82 (3H, s), 0.90 (3H, s), 0.96 (3H, s), 0.99 (3H, s), 5.04 (1H, t, J = 7.3 Hz, H-24), 9.46 (1H, d, J = 7 Hz, H-21); 13C NMR (CDCl3, 125 MHz) δ: 35.58 (t, C-1), 27.81 (t, C-2), 78.90 (d, C-3), 38.87 (s, C-4), 50.38 (d, C-5), 19.11 (t, C-6), 29.19 (t, C-7), 134.76 (s, C-8), 133.93 (s, C-9), 37.07 (s, C-10), 20.75 (t, C-11), 26.43 (t, C-12), 44.31 (s, C-13), 49.47 (s, C-14), 30.60 (t, C-15), 29.66 (t, C-16), 45.35(d, C-17), 16.82 (q, C-18), 18.18 (q, C-19), 55.43 (d, C-20), 206.09 (d, C-21), 26.81 (s, | 5.5 kg of dry and pulverized sporophore of Inonotus obliquus was immersed in ethyl acetate at room temperature for 4 hours with 22 L solvent each time. The total solvent was extracted three times. The extract solution was collected and concentrated under reduced pressure to afford an ethyl acetate extract. 120 g of ethyl acetate extract was subjected to normal phase silica gel column chromatography, the chloroform and methanol were used as eluent, gradient elution was used and TLC was used to analyse, and nine components |
| | | C-22), 25.75 (t, C-23), 123.54 (d, | (Fri to 9) were combined. The |
| | | C-24), 132.41 (s, C-25), 25.63 (q, | component Fr3 was subjected to normal |
| | | C-26), 17.68 (q, C-27), 15.38 | phase silica gel column chromatography |
| | | (q, C-28), 27.95 (q, C-29), 24.17 | with petroleum ether-ethyl acetate |
| | | (q, C-30). | gradient elution (0-100%), TLC analyse |
| | | | and separation. Five components (Fr3-1 |
| | | | to 3-5) were combined and Compound |
| | | | 2″ (4.5 mg) was obtained. |
| 3″ 4″ | 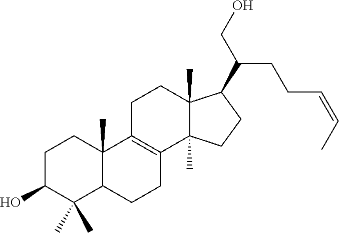 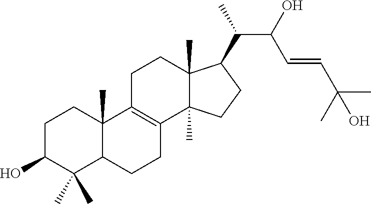 | 1H-NMR (600 MHz, CDCl3) δ: 5.19 (1H, t, J = 7.2 Hz, H-24), 3.67 (1H, dt, J = 6.3, 3.3 Hz, H-22), 3.24 (1H, dd, J = 11.3, 4.6 Hz, H-3), 1.76 (3H, brs, H-27), 1.66 (3H, br s, H-26), 0.95 (3H, d, J = 6.6 Hz, H-20); 13C-NMR (150 MHz, CDCl3) δ: 135.1 (C-25), 134.6 (C-8), 134.2 (C-9), 121.3 (C-24), 79.0 (C-3), 73.4 (C-22), 50.4 (C-5), 49.4 (C-14), 47.2 (C-17), 44.8 (C-13), 41.7 (C-20), 38.9 (C-4), 37.0 (C-10), 35.6 (C-1), 30.9 (C-15), 30.9 (C-15), 29.1 (C-7), 27.9 (C-2), 27.8 (C-19), 27.2 (C-23), 26.5 (C-12), 26.0 (C-26), 24.3 (C-30), 21.0 (C-11), 19.1 (C-6), 18.2 (C-19), 18.0 (C-27), 15.7 (C-18), 15.4 (C-28), 12.6 (C-21). 1H-NMR (600 MHz, CDCl3) δ 5.79 (1H, d, H-24), 5.71 (1H, dd, J = 7.0, 16.1 Hz, H-23), 4.23 (1H, dd, J = 3.4, 7.0 Hz, H-22), 3.24 (1H, dd, J = 11.7, 4.6 Hz, H-3), 2.02 (2H, m, H-11), 1.36 (3H, s, H-27), 1.35 (3H, s, H-26), 1.00 (3H, s, H-28), 0.83 (3H, s, H-30), 0.81 (3H, s, H-29); 13C-NMR (150 MHz, CDCl3) δ 135.7 (C-24), 134.4 (C-9), 134.1 (C-8), 129.4 (C-23), 82.5 (C-25), 78.9 (C-3), 74.4 (C-22), 50.3 (C-5), 49.4 (C-14), 47.6 (C-17), 44.7 (C-13), 42.6 (C-20), 38.8 (C-4), 37.0 (C-10), | 2.0 kg of Inonotus obliquus was extracted with 5.5 L methanol at room temperature for 80 hours after pulverizng and grinding and filtered. Repeat four times. The extract solution was combined and concentrated to a small volume, suspended in water, and extracted by n-hexane for four times. The extract solution was concentrated to a constant weight under reduced pressure, and 25.0 g of n-hexane extract was obtained. 25.0 g n-hexane extract was separated by silica gel column chromatography eluted with hexane (V)/ethyl acetate (V) = 95/5 (7.0 L), 8/2 (7.0 L), 5/5 (5.0 L) and ethyl acetate 100% (6.0 L). The same fraction was combined according to the results of TLC tracking analysis, nine components (H1-H9) were obtained by concentration. H5 (6.5 g) was separated and purified by silica gel column chromatography and HPLC to afford compound 3″ (30 mg) and compound 4″ (3.8 mg). |
| | | 35.5 (C-1), 30.9 (C-12), 30.9 (C-15), | |
| | | 27.9 (C-28), 27.8 (C-2), 27.4 (C-16), | |
| | | 26.5 (C-7), 24.4 (C-27), 24.3 (C-26), | |
| | | 24.1 (C-30), 20.9 (C-11), 19.1 (C-19), | |
| | | 18.2 (C-6), 15.8 (C-18), 15.4 (C-29), | |
| | | 12.5 (C-21). | |
| 5″ | 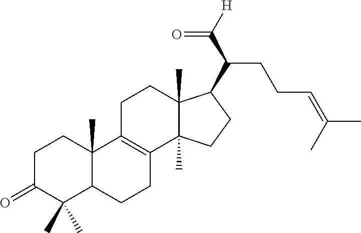 | 1H NMR (CDCl3): 1.95, 1.59 (2H, m), 2.58, 2.37 (2H, m), 1.59 (1H, m), 1.59 (2H, m), 1.99, 1.41 (2H, m), 2.04, 1.98 (2H, m), 1.62, 1.32 (2H, m), 1.63 (2H, m), 1.54 (2H, m), 2.06 (1H, m), 0.7 (3H, s), 1.09 (3H, s), 2.22 (1H, m), 9.45 (1H, d, J = 5.6 Hz), 2.58, 2.39 (2H, m), 1.88, 1.67 (2H, m), 5.34 (1H, t, J = 6.0 Hz), 1.67 (3H, s), 1.56 (3H, s), 1.06 (3H, s), 1.08 (3H, s), 0.91 (3H, s). 13C NMR (CDCl3): 36.0 (C-1), 34.6 (C-2), 217.8 (C-3), 47.4 (C-4), 51.1 (C-5), 19.3 (C-6), 26.2 (C-7), 134.8 (C-8), 133.4 (C-9), 36.9 (C-10), 20.8 (C-11), 29.6 (C-12), | 21 kg of Inonotus Obliquus was pulverized and extracted with 35 L of 95% ethanol at room temperature for 50 h, and 25 L of 95% ethanol was added, the mixture was refluxed at 80° C. twice for 3.5 hours each time. The solvent was recovered to obtain an ethanol extract. The extract was cold-extracted with 15 L 80% acetone for 54 hours and then evaporated to obtain acetone extract. The mixture was dispersed in an appropriate amount of distilled water, and extracted with petroleum ether (3 L × 3), and concentrated to afford petroleum ether extract (132 g). |
| | | 44.2 (C-13), 49.5 (C-14), 30.6 (C-15), | The petroleum ether extract (100 g) was |
| | | 29.1 (C-16), 45.2 (C-17), 16.9 (C-18), | subjected to silica gel column |
| | | 18.6 (C-19), 55.4 (C-20), 206.2 (C-21), | chromatography, and eluted with |
| | | 34.6 (C-22), 25.7 (C-23), 123.4 (C-24), | petroleum ether-ethyl acetate gradient |
| | | 132.5 (C-25), 17.7 (C-26), 25.7 (C-27), | solvent (50:1, 30:1, 15:1, 10:1, 4:1, 1:1) |
| | | 26.1 (C-28), 21.3 (C-29), 24.2 (C-30). | and the same fraction was combined by |
| | | | thin layer chromatography to afford 14 |
| | | | fractions (Fr. A-N). Among them, Fr. F |
| | | | (5.3 g) was subjected to silica gel |
| | | | column chromatography and eluted with |
| | | | petroleum ether-ethyl acetate gradient |
| | | | solvent (60:1→1:1) to obtain 7 fractions |
| | | | Fr. Fl-F7. Fr. F7 (420 mg) was further |
| | | | subjected to gel Sephadex LH-20 |
| | | | chromatography (mobile phase was |
| | | | chloroform-methanol 3:2) and reversed |
| | | | phase silica gel column chromatography |
| | | | (80% methanol→100% methanol) to |
| | | | afford compound 5″ (20 mg). |
| 6″ 7″ 8″ | 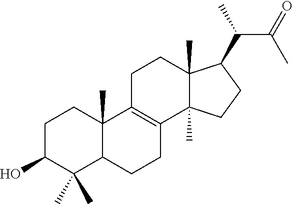 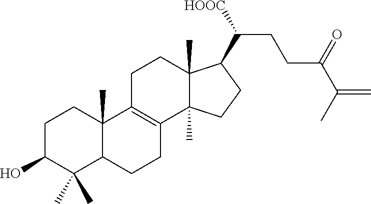 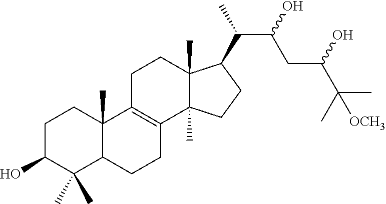 | 1H NMR (400 Hz, CD3OD): 1.75, m; 1.22, m; 1.61 (2H); 3.13, m; 1.03, br.d (10.4); 1.71, m; 1.53, m; 2.06 (2H); 2.07 (2H); 1.85, m; 1.66, m; 1.20, m; 1.79, m; 0.75, s; 1.00, s; 2.56, m; 1.08, d (7.2); 2.11, s; 0.96, s; 0.78, s; 0.92, s. 13C NMR (100 Hz, CD3OD): 37.0 (C-1), 28.5 (C-2), 79.7 (C-3), 40.0 (C-4), 52.0 (C-5), 19.5 (C-6), 27.7 (C-7), 135.6 (C-8), 136.2 (C-9), 38.3 (C-10), 22.1 (C-11), 32.4 (C-12), 46.0 (C-13), 50.7 (C-14), 32.2 (C-15), 28.3 (C-16), 48.2 (C-17), 16.7 (C-18), 19.7 (C-19), 52.1 (C-20), 17.0 (C-21), 216.1 (C-22), 28.5 (C-23), 28.7 (C-28), 16.2 (C-29), 24.7 (C-30). 1H NMR (400 Hz, CD3OD): 1.70, m; 1.17, m; 1.56 (2H); 3.10, dd (9.2, 7.2); 1.00, br, d (10.4); 1.67, m; 1.50, m; 2.03 (2H); 1.96 (2H); 1.64, m; 1.48 m; 1.60, m; 1.18, m; 1.94, m; 1.61, m; 2.03, m; 0.74, s; 0.95, s; 2.21, m; 1.80, m; 1.56, m; 2.65 (2H); 5.99, br, s; 5.77, br, s; 1.78, s; 0.93, s; 0.75, s; 0.87, s. 13C NMR (100 Hz, CD3OD): 37.1 (C-1), 28.6 (C-2), 79.7 (C-3), 40.0 (C-4), 52.0 (C-5), 19.5 (C-6), 27.7 (C-7), 135.6 (C-8), 136.1 (C-9), 38.3 (C-10), 22.0 (C-11), 30.1 (C-12), 45.6 (C-13), 50.7 (C-14), 31.6 (C-15), 28.0 (C-16), 48.6 (C-17), 16.6 (C-18), 19.7 (C-19), 49.1 (C-20), 180.7 (C-21), 28.4 (C-22), 36.2 (C-23), 204.0 (C-24), 145.7 (C-25), 125.9 (C-26), 17.7 (C-27), 28.7 (C-28), 16.2 (C-29), 24.8 (C-30) 1H NMR (400 Hz, CDCl3): 1.74, m; 1.23, m; 1.66, m; 1.58, m; 3.24, dd (11.6, 4.4); 1.05, br, d (10.4); 1.69, m; 1.52, m; 2.04 (2H); 2.03 (2H); 1.71 (2H); 1.64, m; 1.21, m; 1.73, m; 1.47, m; 0.73, s; 0.98, s; 1.75, m; 0.93, d (6.8); 3.91, dd (10.0, 2.4); 1.48, m; 1.34, m; 3.65, dd (10.4, 1.6); 1.13, s; 1.00, s; 0.81, s; 0.87, s; 3.24, s. 13C NMR (100 Hz, CDCl3): 35.6 (C-1), 27.8 (C-2), 79.0 (C-3), 38.9 (C-4), 50.4 (C-5), 18.2 (C-6), 26.5 (C-7), 134.2 (C-8), 134.6 (C-9), 37.0 (C-10), 21.0 (C-11), 30.9 (C-12), | The crude powder of Inonotus Obliquus (8 kg) was extracted with 95% ethanol (40 L) 5 times at 1.5 hours each time, and the solvent of the extract was removed under reduced pressure to afford 350 g of ethanol extract. The ethanol extract was suspended in water (14 L) and extracted with petroleum ether and ethyl acetate (14 L * 2) successively, and the solvent of the extract was removed under reduced pressure to afford petroleum ether extract and ethyl acetate extract. The ethyl acetate extract was subjected to silica gel column chromatography and eluted with petroleum ether-ethyl acetate gradient solvent (25: 1-10: 1-5: 1-1:1, each eluent was 2 L). The methanol was use to rinse to afford 10 fractions Fr.1-Fr.10. Fr.5 (1.1 g) was subjected to silica gel column chromatography with chloroform-acetone gradient elution (20:1-15:1); reverse silica gel chromatography with methanol-water elution (67:33-100:0); silica gel column chromatography with chloroform-methanol gradient elution (50:1-30:1-10:1); and reversed phase silica gel chromatography with methanol-water (80:20), and then Compound 6″ (3.02 mg) was obtained. Fr. 7 (2.75 g) was subjected to reverse phase silica gel chromatography with methanol-water gradient elution (50:50-80:20) and finally the methanol was used to rinse. Five fractions of Fr.7.1-Ff7.5 was obtained. Fr. 7.5 was subjected to silica gel column chromatography with chloroform-methanol-water gradient elution (13:0.95:0.05-7:0.95:0.05); and then subjected to reversed-phase high performance liquid chromatography. The fraction of methanol-water (83:17) eluent was subjected to silica gel column chromatography. The eluent was chloroform-methanol-water (13:0.95:0.05), and compound 7″ (3.13 mg) was obtained. Fr. 3 (5.47 g) was subjected to silica gel column chromatography with petroleum |
| | | 44.8 (C-13), 49.4 (C-14), 30.9 (C-15), | ether-ethyl acetate 7:1-3:1-1:1, and |
| | | 27.2 (C-16), 47.4 (C-17), 15.8 (C-18), | three fractions of Fr.3.1-Fr3.3 were |
| | | 19.1 (C-19), 41.7 (C-20), 12.9 (C-21), | obtained. Fr.3.1 was was subjected to |
| | | 74.7 (C-22), 30.0 (C-23), 78.9 (C-24), | reversed-phase silica gel |
| | | 77.1 (C-25), 18.9 (C-26), 20.5 (C-27), | chromatography, the eluent was |
| | | 27.9 (C-28), 15.4 (C-29), 24.3 (C-30) | methanol-water (70:30-76:24), and the |
| | | | obtained third fraction Fr.3.1.3 was |
| | | | further separated by reversed phase |
| | | | silica gel, the eluent wsa methanol-water |
| | | | (80:20-90:10), four fractions of |
| | | | Fr.3.1.3.1-Fr.3.1.3.4 were obtained. Fr. |
| | | | 3.1.3.1 was subjected to silica gel |
| | | | column chromatography, the eluent was |
| | | | chloroform-acetone (19:1-8:1), and |
| | | | compound 8″ (0.23 mg) was obtained. |
| 17″ | 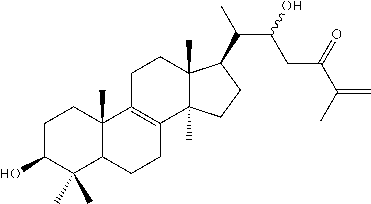 | 1H NMR (Pyridine-d5): 1.74, m, 1.24, 1.68 (2H), 3.23, dd (4.4, 11.2), 1.05, m, 1.70, 1.53, m, 1.70, 1.45, m, 2.03 (2H), 1.68, 1.24, 1.40, m, 0.74, s, 0.98, s, 1.83, m, 0.96, d (6.8), 4.17, m, 2.78, d (17.2), 2.66 dd (10.4, 17.2), 6.02, s, 5.85, s, 1.89, s, 0.99, s, 0.81, s, 0.88, s. 13C NMR (Pyridine-d5): 35.5 (C-1), 27.8 (C-2), 78.9 (C-3), 38.8 (C-4), 50.3 (C-5), 18.2 (C-6), 27.2 (C-7), 134.5 (C-8), 134.1 (C-9), 37.0 (C-10), 20.9 (C-11), 26.4 (C-12), 44.8 (C-13), 49.4 (C-14), 30.9 (C-15), 30.9 (C-16), | Inonotus obliquus (20 kg) was refluxed with 95% ethanol 3 times at 2.5 hours each time, and solvent of the extract was removed under reduced pressure to afford ethanol extract (650 g). The ethanol extract was suspended in chloroform-water (1:1) to afford chloroform extract (400 g) and an aqueous phase. The aqueous phase was extracted with ethyl acetate and the solvent was removed under reduced pressure and 40g of ethyl acetate extract was obtained. The ethyl acetate extract |
| | | 47.2 (C-17), 15.7 (C-18), 19.1 (C-19), | was subjected to silica gel column |
| | | 41.1 (C-20), 12.9 (C-21), 69.3 (C-22), | chromatography eluted with petroleum |
| | | 37.2 (C-23), 203.4 (C-24), 144.7 | ether-acetone gradient solvent |
| | | (C-25), 125.8 (C-26), 17.4 (C-27), | (50:1-25:1-20:1-15:1-9:1-8:2-7:3-6: |
| | | 15.4 (C-28), 27.9 (C-29), 24.3 (C-30) | 4-1:1-0:1), ten fractions Fr.1-Fr.10 were |
| | | | obtained. Each fraction was further |
| | | | subjected to normal phase silica gel |
| | | | chromatography, eluted with the eluents |
| | | | in the order of increaseing polarity: |
| | | | petroleum ether-acetone, |
| | | | chloroform-acetone or |
| | | | chloroform-methanol. Fr. 6 (2.3 g) was |
| | | | purified by preparative high |
| | | | performance liquid chromatography |
| | | | (methanol-water-acetic acid, 90:10:1) to |
| | | | afford compound 17″ (2.4 mg). |
| 20″ | 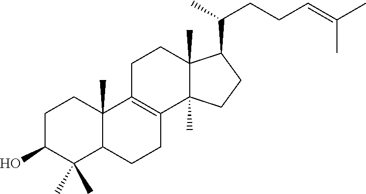 | 1H NMR (CDCl3, 400 MHz): 0.73, 0.82, 0.88, 0.99, 1.01, 1.66, 1.76 (each, 3H, s), 0.95 (3H, d, J = 6.8 Hz), 3.68 (1H, m), dH 3.24 (1H, dd, J = 9.2, 4.4 Hz), 5.19 (1H, t, J = 6.8 Hz). 13C NMR (CDCl3, 100 MHz): 35.6 (C-1), 27.8 (C-2), 79.0 (C-3), 38.9 (C-4), 50.4 (C-5), 19.2 (C-6), 26.5 (C-7), 134.5 (C-8), 134.2 (C-9), 37.0 (C-10), 21.0 (C-11), 29.1 (C-12), 44.8 (C-13), 49.4 (C-14), 30.9 (C-15), 30.9 (C-16), 47.2 (C-17), 15.7 (C-18), 18.2 | 250 kg of crude powder of dried sporophore of Inonotus obliquus was extracted with ethanol by refluxing, and the solvent of the extract was removed under reduced pressure to obtain 300 g of ethanol extract. The ethanol extract was suspended in water and extracted with petroleum ether, and the extract was concentrated under reduced pressure to afford petroleum ether extract (29 g). The petroleum ether extract was |
| | | (C-19), 41.6 (C-20), 12.6 (C-21), 73.4 | subjected to silica gel column |
| | | (C-22), 27.2 (C-23), 121.4 (C-24), | chromatography eluted with petroleum |
| | | 135.2 (C-25), 26.0 (C-26), 18.0 (C-27), | ether-ethyl acetate gradient solvent |
| | | 28.0 (C-28), 15.4 (C-29), 24.3 (C-30). | (100:1-10:1), the mixture I was obtained |
| | | 13C NMR (CD3OD, 100 MHz): 37.0 | by eluting with petroleum ether-ethyl |
| | | (C-1), 28.5 (C-2), 79.7 (C-3), 40.0 | acetate 10:1. The mixture I was |
| | | (C-4), 52.0 (C-5), 19.5 (C-6), 27.7 | subjected to column chromatography |
| | | (C-7), 135.6 (C-8), 136.2 (C-9), 38.3 | eluted with petroleum ether-ethyl |
| | | (C-10), 22.1 (C-11), 32.1 (C-12), 46.0 | acetate gradient solvent (15:1-1:1), |
| | | (C-13), 50.7 (C-14), 32.3 (C-15), 30.2 | lanosterol 20″ (0.53 g) was obtained by |
| | | (C-16), 48.4 (C-17), 16.2 (C-18), 19.7 | eluting with petroleum ether-ethyl |
| | | (C-19), 44.2 (C-20), 13.1 (C-21), 74.9 | acetate 5:1. Among them, 50 mL of the |
| | | (C-22), 26.1 (C-23), 123.5 (C-24), | eluent was collected each time, and the |
| | | 133.2 (C-25), 26.1 (C-26), 18.0 (C-27), | same fraction was combined according |
| | | 28.7 (C-28), 16.2 (C-29), 24.7 (C-30). | to the TLC detection result, and the |
| | | | single component was recrystallized. |

### Example 21

In this example, apart of the specific compounds obtained in Example 19 and Example 20 were selected for intracellular mutated lens protein (αB-crystallin R120G) aggregation inhibition experiment (experimental method was described in Chinese patent CN201580057679). The aggregation inhibition activities of compound I and compound II in the present invention on lens protein mutants were evaluated to screen a compound which was effective in treating related disease such as cataract.

### Experimental Method

The tumor cell line HeLa was selected as a host cell transfected with the mutant lens protein (αB-crystallin R20G) expression plasmid, the medium was DMEM with 1000FBS, and the culture conditions were 37° C., saturated humidity and 5% carbon dioxide cell incubator. Mycoplasma was negative. The transfection process is that the appropriate number of HeLa cells were inoculated on the pre-coated slides. When fully adherent cells grew to about 75% cell density, the expression vector was transfected with liposome. After 4 hours of transfection, the medium was changed to normal medium. Then the HeLa cells were cultured in the cell incubator for 13 hours to induce the expression of exogenous mutant lens protein. After changing the medium, the replaced new medium contained various compounds of the formula I and formula II of the present invention to be tested (DMSO was used as solvent in all mother fluid), wherein the final concentration of each compound was 10 μM, the negative control was 1.5% DMSO solvent, and the positive control was lanosterol (10 μM). The HeLa cells were treated with the medium containing the test compound, 1.5% DMSO solvent and lanosterol for 3.5 hours, respectively, and the normal fresh medium containing no DMSO, test compound or lanosterol was exchanged and the cells were cultured for another 6.5 h, then Anti-p62 antibody and DAPI were used for routine cellular immunofluorescence detection. The field of vision of three positions per slide was randomly selected, the ratio of cells containing aggregated crystalline proteins (lens protein aggregates) was calculated, and the effect of each test compound on intracellular aggregation of mutant lens proteins was finally determined, the results were shown in Table 19.

TABLE 19
The effect of the different Compounds of Formula I and Formula II as well as inonotus
obliquus extract on intracellular aggregation of lens protein mutant (alpha B-crystallin R120G)

| | Inhibitory | | Inhibitory | | Inhibitory |
|---|---|---|---|---|---|
| Compound No. | effect | Compound No. | effect | Compound No. | effect |
| Compound 9 | ++ | Compound 10 | + | Compound 5 | + |
| Compound 17 | ++ | Compound 13 | ++ | Compound 15 | + |
| Compound 23 | + | Compound 21 | + | Compound 20 | ++ |
| Compound 24 | + | Compound 25 | + | Compound 27 | + |
| Compound 32 | + | Compound 34 | + | Compound 42 | + |
| Compound 15′ | + | Compound 20′ | ++ | Compound 21′ | ++ |
| Compound 2″ | ++ | Compound 3″ | ++ | Compound 4″ | + |
| Compound 5″ | ++ | Compound 6″ | ++ | Compound 7″ | ++ |
| Compound 8″ | ++ | Compound 17″ | ++ | Trametenolic acid | + |
| Inotodiol | + | negative control | ++++ | lanosterol | +++ |

Note:
the correspond relationship between the symbols used for inhibitory effect in the table and percentage of cells containing aggregated proteins was as follows:
″+″ represented that <30% of the cells in the field of vision contained protein aggregates.
″++″ represented that >30% and <45% of the cells in the field of vision contained protein aggregates.
″++″ represented that >45% and <60% of the cells in the field of vision contained protein aggregates.
″+++″ represented that >60% of the cells in the field of vision contain protein aggregates.

Table 19 showed that in addition to inotodiol and trametenolic acid, the other compounds of formula I and formula II as well as the inonotus obliquus extract were also better than lanosterol in reducing intracellular lens protein aggregation.

### Example 22

Low Concentrations of Compound of Formula I was Effective in Dissolving Lens Protein Aggregates

In this example, it was investigated whether lower concentration of compound of formula I had significant depolymerization effect on higher concentrations of protein aggregates.

The experimental method was as follows: Inotodiol was used as test compound, and its dissolution effect on wild-type αB lens protein aggregate (WT αB) was measured, and lanosterol was used as positive control.

In the present example, the formulations of pharmaceutical composition in the test group and the control group were liposome, wherein the component of the formulation of test group was 40 mg of DPPC, 10 mg of cholesterol and 10 mg of test compound; the component of the formulation of positive control group was 40 mg of DPPC, 10 mg of cholesterol and 10 mg of lanosterol; the component of formulation of the blank control group was 40 mg of DPPC and 10 mg of cholesterol.

The liposome formulation was prepared as follows: 40 mg of DPPC, 10 mg of cholesterol and 10 mg of corresponding compound were weighed and placed in a pear-shaped bottle, and the blank liposome only contained 40 mg of DPPC and 10 mg of cholesterol. An appropriate amount of chloroform was added to sufficiently dissolve the mixture of DPPC, cholesterol and corresponding compound, and an appropriate amount of glass beads was added, followed by rotary evaporation under vacuum at 50° C. After the solution was evaporated, the mixture is further evaporated for 10 minutes to obtain dry film, then the dry film was placed at room temperature overnight in a vacuum drying oven to sufficiently evaporate the organic solvent. The next day, 10 ml of the preheated PBS solution was added, and the liposome film was rotationally hydrated in water bath at 60° C. After the film was completely detached from the vessel, the rotation was continued for 1 h. The hydration solution was placed in ice bath and sonicated for 40 min. The supernatant was obtained by centrifuging at 12,000 rpm for 10 min at 4° C. to obtain a liposome solution of the corresponding compound.

The analysis of compound concentration in the liposome formulation: The obtained liposome solution was demulsificated with 4 to 5 volumes of methanol solution to afford clear solution. The concentration of compound in the liposome was determined by HPLC method, and the chromatographic conditions are as follows. RP C18 (4.6 mm×150 mm, 5 m) chromatographic column was used, column temperature was 35° C., detection wavelength was 210 nm, flow rate was 1.0 ml/min, and injection volume was 25 μl. In the analysis of lanosterol liposome samples and their standards, methanol was used as mobile phase, and in the analysis of inotodiol and its standards, methanol:water (90:10) was used as mobile phase. After the comparative analysis, the concentration of corresponding compound in the liposome preparation was determined.

The induction of protein aggregates is as follows. A solution containing 1.2 mg/ml of wild-type αB lens protein was incubated at 37° C. for 48 hours, and 0.02% NaN₃ was added to the system to prevent bacterial infection, thereby forming αB lens protein aggregates (W αB). Then an inotodiol formulation or lanosterol formulation (liposome form, the final concentration of both compounds was 125 μM) was added to the solution respectively. The solution was kept for 24 hours. The control group was treated with blank liposomes for the same treatment. After the treatment, ThT fluorescence was used to detect the effect of different compounds on αB lens protein aggregates.

The results were shown in FIG. 33, inotodiol was able to significantly reduce the ThT fluorescence signal, while lanosterol increased the ThT fluorescence signal. The result suggested that inotodiol could extremely efficiently re-dissolve αB lens protein aggregates at low concentration or closer to body level of compound concentration (*: p<0.05 compared to the blank control). The effect was much better than lanosterol. This demonstrated that the compounds of formula I of the present invention, such as inotodiol, had better drug development prospects for cataract treatment/prevention.

All documents mentioned in the present invention are incorporated herein by reference, as if each document were individually recited for reference. It should be understood that those skilled in the art will be able to make various changes or modifications to the present invention after reading the teachings of the present invention, which also fall within the scope of the claims appended hereto.

1-37. (canceled)

38. An ophthalmic composition, comprising (a) a compound of formula I, or an optical isomer, racemate, solvate, pharmaceutically acceptable salt, prodrug, or deuterated Compound thereof; 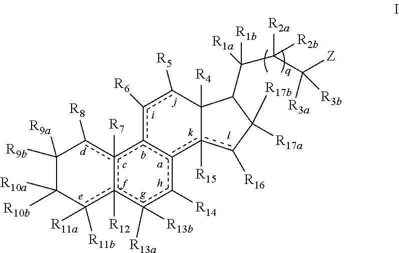
wherein,
q is 0, 1 or 2;
R1a, R1b, R2a, R2b, R3a and R3b are each independently selected from hydrogen, substituted or unsubstituted C1-C20 alkyl, substituted or unsubstituted C2-C20 alkenyl, substituted or unsubstituted C2-C20 alkynyl, substituted or unsubstituted C3-C10 cycloalkyl, —OH, substituted or unsubstituted C1-C10 alkoxy, —COH, —CHO, substituted or unsubstituted C1-C10 ester group, —SH, substituted or unsubstituted C1-C10 alkylthio, -A-B,
or R1a and Rb, R2a and R2b, and/or R3a and R3b constitute ═O;
wherein, A is absent or a divalent linking group; and B is H, —OH, —SH, C1-C3 alkoxy, C1-C3 alkylthio, —CHO, —COOH, C1-C4 ester group, C3-C10 cycloalkyl, aryl, C3-C10 5-6 membered heteroaryl, or benzyl;
with the proviso that at least one of R1a, R2a, R3a, R1b, R2b and R3b is a group containing O or S;
Z is selected from the group consisting of H, substituted or unsubstituted C1-C20 alkyl, substituted or unsubstituted C2-C20 alkenyl, substituted or unsubstituted C2-C20 alkynyl, substituted or unsubstituted C3-C10 cycloalkyl, substituted or unsubstituted aryl, substituted or unsubstituted 5-8 membered heteroaryl, substituted or unsubstituted —(C1-C6 alkylene)-aryl, substituted or unsubstituted-(C1-C6 alkylene)-5-8 membered heteroaryl, —OH, substituted or unsubstituted C1-C20 alkoxy, —SH, substituted or unsubstituted C1-C20 alkylthio, substituted or unsubstituted C1-C10 ester group, substituted or unsubstituted C1-C10 acyl, and substituted or unsubstituted —O-aryl;
R4 is hydrogen, substituted or unsubstituted C1-C4 alkyl;
R7, R12 and R15 are each independently selected from absent, hydrogen, substituted or unsubstituted C1-C4 alkyl;
R13a and R13b are each independently selected from absent, hydrogen, substituted or unsubstituted C1-C8 alkyl, —OH, substituted or unsubstituted C1-C8 alkoxy, —SH, substituted or unsubstituted C1-C8 alkylthio, halogen, substituted or unsubstituted C1-C3 acyl, or R13a and R13b constitute ═O, and at most one of R13a and R13b is absent;
R11a and R11b are each independently selected from absent, hydrogen, substituted or unsubstituted C1-C6 alkyl, —OH, or R1a and R11b constitute ═O, and at most one of R11a and R11b is absent;
R10a and R10b are each independently selected from hydrogen, —OH, substituted or unsubstituted C1-C8 alkoxy, —SH, substituted or unsubstituted C1-C8 alkylthio, —OSO₃H, —OCO-substituted or unsubstituted C1-C7 alkyl, —OPO₃H, —COOH, -(substituted or unsubstituted C1-C7 alkylene)-COOH, —CHO, -(substituted or unsubstituted C1-C7 alkylene)-CHO, or R10a and R10b together constitute ═O; and
R5, R6, R8, R9a, R9b, R14, R16, R17a and R17b are each independently selected from the group consisting of hydrogen, OH, substituted or unsubstituted C1-C8 alkyl, substituted or unsubstituted C1-C8 alkoxy, and halogen;
the “substituted” means that one or more hydrogen atoms on the group are substituted by group selected from the group consisting of halogen, —OH, —SH, —COOH, —(C1-C7 alkylene)-COOH, ═O, —CHO, —(C1-C7 alkylene)-CHO, C1-C7 alkyl-OCO—, C1-C3 alkyl, C3-C6 cycloalkyl, NRaRb, wherein Ra and Rb are each independently H, C1-C6 alkyl, C1-C6 haloalkyl, C3-C6 cycloalkyl, or benzyl.

39. The ophthalmic composition of claim 38, wherein the divalent linking group has 1-10 linking units selected from the group consisting of —CRaRb—, —C(OH)Ra—, —NRa—, —O—, —CO—, wherein, Ra and Rb are each independently selected from H, C1-C6 alkyl, C1-C6 haloalkyl, C3-C6 cycloalkyl, or benzyl;
R1a, Rb, R2a, R2b, R3a and R3b are each independently selected from hydrogen, —OH, —CHO, —COOH, —SH, substituted or unsubstituted C1-C3 alkyl-OCO—, substituted or unsubstituted C1-C4 alkyl, -A-B, or Ra and R1b, R2a and R2b, and/or R3a and R3b constitute ═O; wherein, A is independently a substituted or unsubstituted C1-C4 alkylene, and B is independently —OH, —SH, C1-C3 alkoxy, —CHO, —COOH, ═O;
Z is (L1)m-L2=Y, m is 0, 1, 2, 3 or 4, and each L1 is independently —CH₂—, —CO—, —O—, —C(C1-C3 alkyl)H—, C(C1-C3 alkyl)₂-, —C(C1-C3 alkyl)O—, or —NH—, L2 is —CH═, or —C(C1-C3 alkyl)=;
Y is ═CH₂, ═CH-substituted or unsubstituted C1-C7 alkyl, ═C-(substituted or unsubstituted C1-C7 alkyl)₂, ═CH-substituted or unsubstituted C2-C7 alkenyl, or ═CH— substituted or unsubstituted C2-C7 alkynyl;
R5, R6, R8, R9a, R9b, R14, R16, R17a and R17b are each independently selected from hydrogen, methyl; and/or
Z is —CH₂C(CH₃)₂—OH.

40. The ophthalmic composition of claim 38, wherein, at least one of R1a and R1b is a group containing O or S; and/or
at least one of R2a and R2b is a group containing O or S; and/or
at least one of R3a and R3b is a group containing O or S.

41. The ophthalmic composition of claim 38, wherein the compound of formula I is a compound of formula I-1: 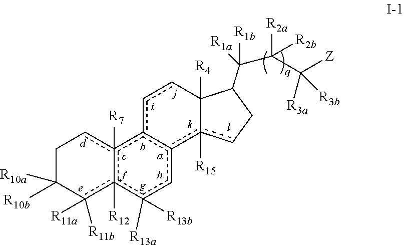
wherein q, R1a, R1b, R2a, R2b, R3a, R3b, R4, R7, R10a, R10b, R11a, R11b, R12, R13a, R13b, R15 and Z are defined as in claim 38;
or the compound of formula I is a compound of formula I-2: 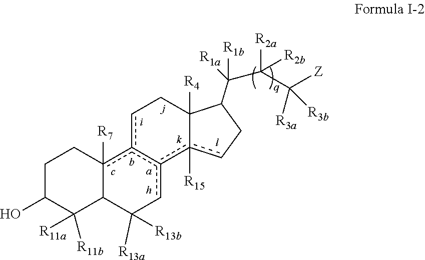
wherein q, R1a, R1b, R2a, R2b, R3a, R3b, R4, R7, R11a, R11b, R13a, R13b, R15 and Z are defined as in claim 38;
or the compound of formula I is selected from the group consisting of: 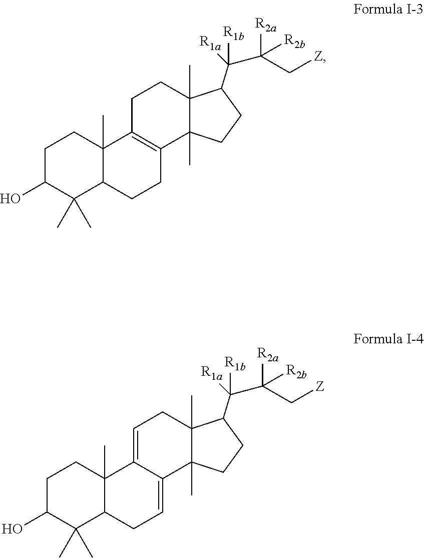
wherein R1a, R1b, R2a, R2b and Z are defined as in claim 38;
or the compound of formula I is selected from the group consisting of: 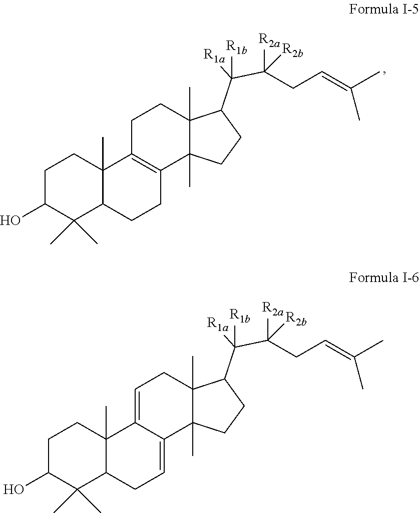
wherein R1a, R1b, R2a, and R2b are defined as in claim 38;
or the compound of formula I is a compound of formula I-7: 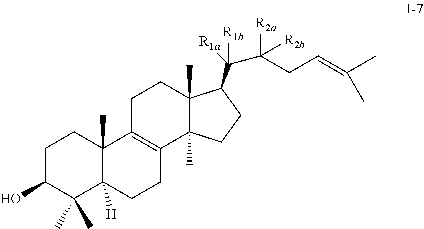
wherein R1a, R1b, R2a, and R2b are defined as in claim 38;
or the compound of formula I is a compound of formula I-8: 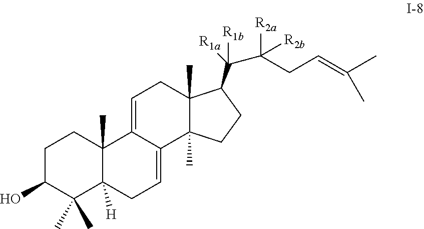
wherein R1a, R1b, R2a, and R2b are defined as in claim 38.

42. The ophthalmic composition of claim 38, wherein the compound of formula I is selected from the group consisting of:

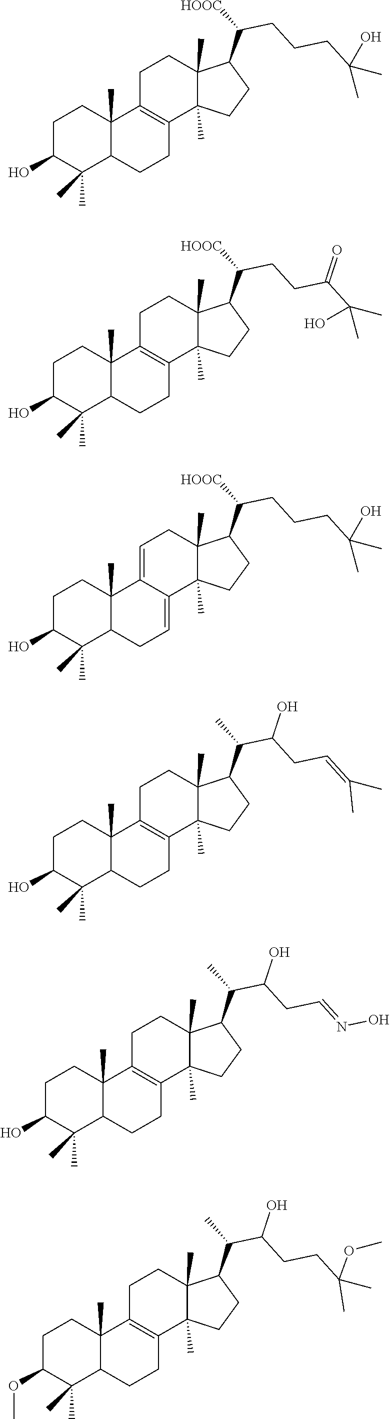

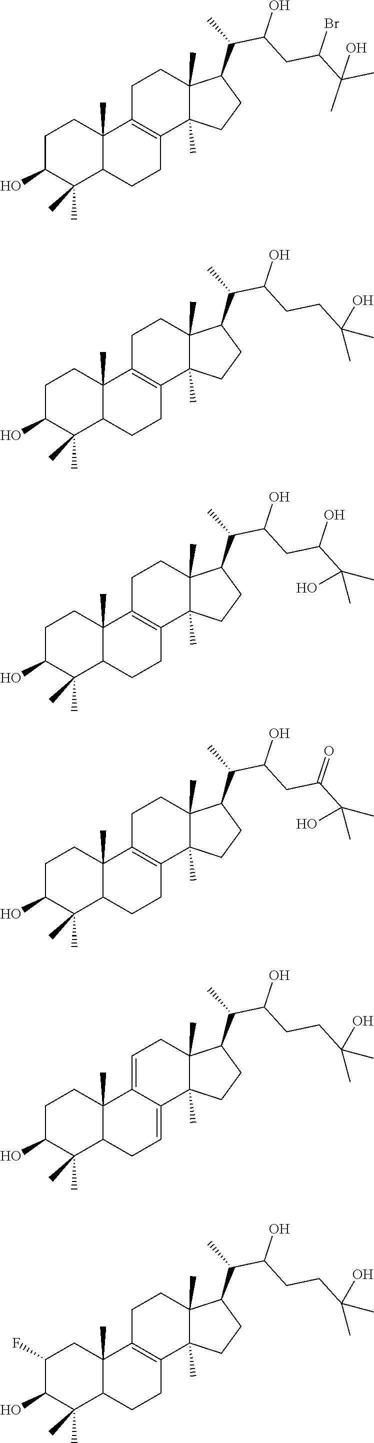

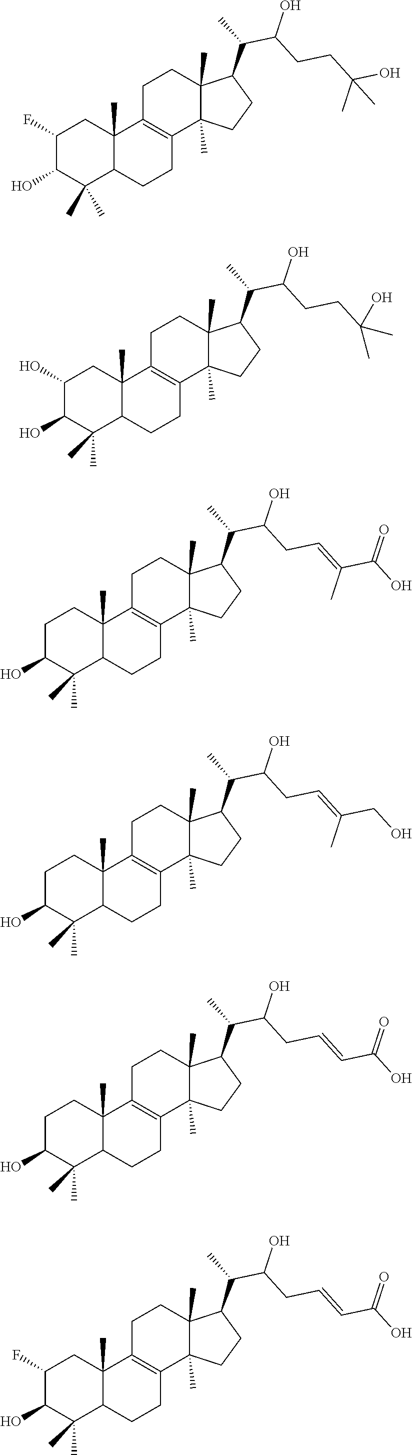

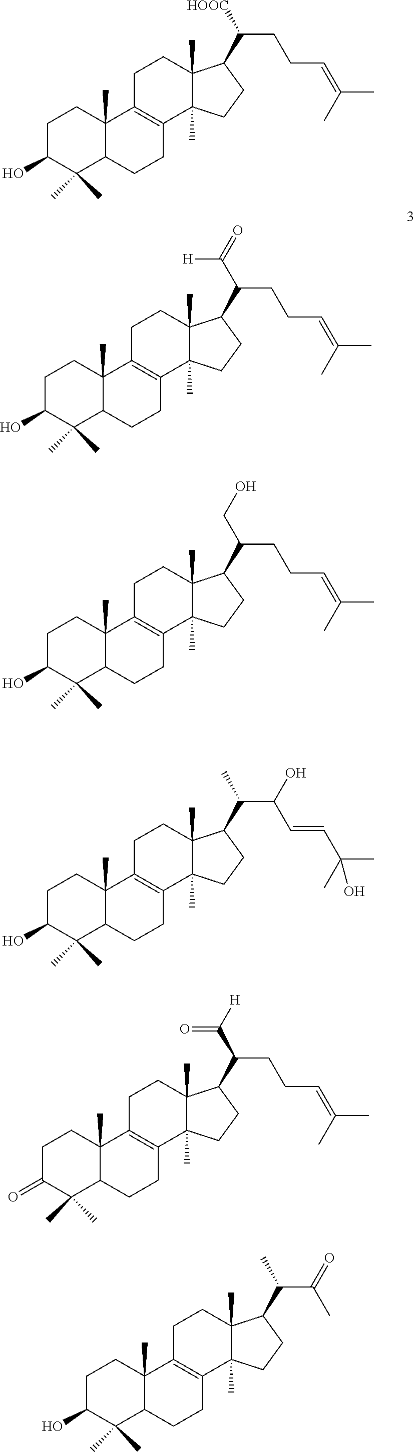

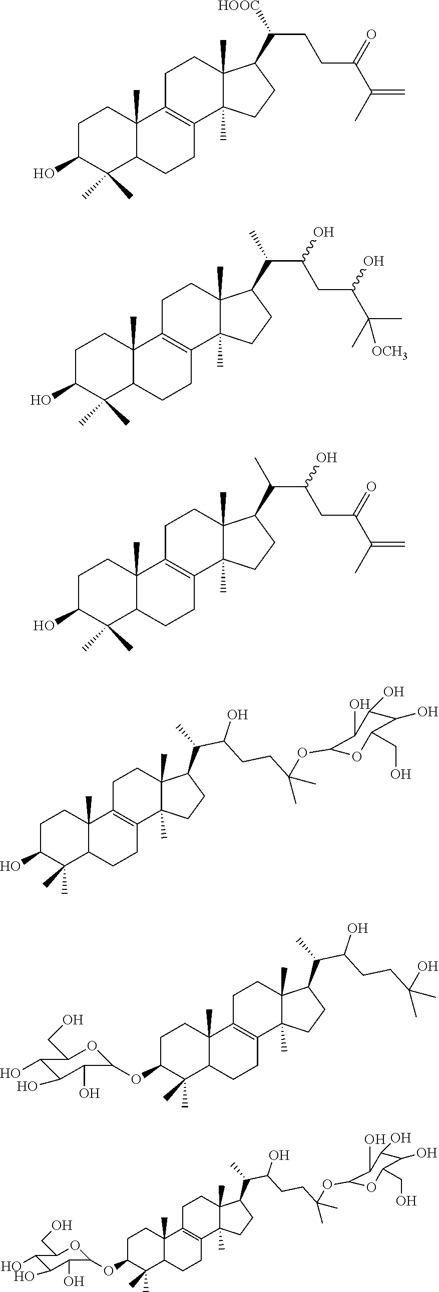

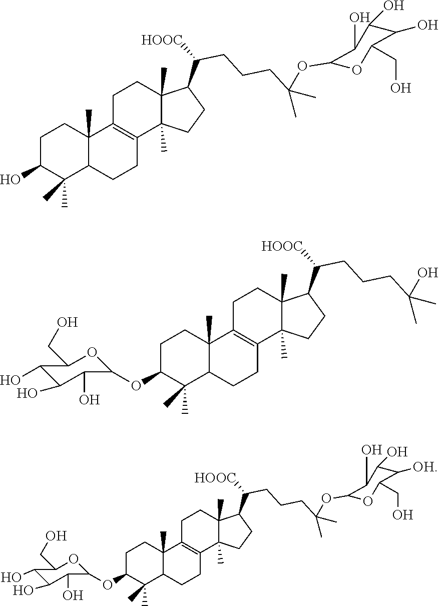

43. The ophthalmic composition of claim 38, wherein the compound is

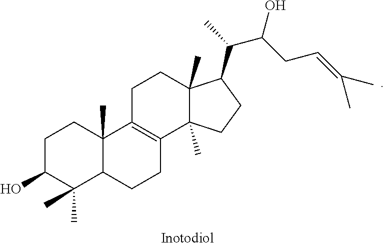

44. The ophthalmic composition of claim 38, wherein the compound of formula I is selected from the group consisting of:

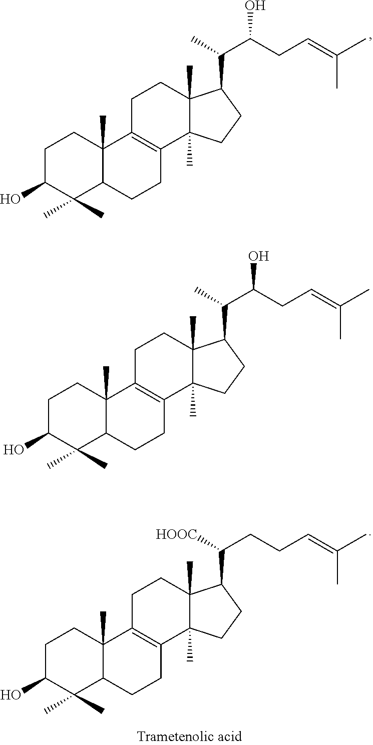

45. The ophthalmic composition of claim 38, wherein the ophthalmic composition further comprises (b) a pharmaceutically acceptable carrier.

46. The ophthalmic composition of claim 38, wherein the dosage form of the ophthalmic composition is intraocular injection preparation.

47. The ophthalmic composition of claim 38, wherein the dosage form of the ophthalmic composition is eye drop, emulsion, gel, eye ointment, sustained release microsphere, intraocular sustained-release implant tablet, medicinal sustained-release film.

48. The ophthalmic composition of claim 38, wherein the ophthalmic composition further comprises (c) a second active ingredient which is selected from the group consisting of lanolin compounds, steroids, terpenoids, azoles, glucocorticoid compounds, antibiotics, or a combination thereof.

49. A method for preventing and/or treating an eye disease caused by lens lesions, which comprises: administering the ophthalmic composition of claim 38 to a subject in need.

50. The method of claim 49, wherein the eye disease caused by lens lesions is selected from the group consisting of cataract, presbyopia, myopia, cortical opacity, presbyopia nuclear sclerosis, and eye complications caused by diabetes.

51. The method of claim 50, wherein the cataract is selected from the group consisting of traumatic cataract, metabolic cataract, senile cataract, congenital cataract, spontaneous cataract, complicated cataract, and a combination thereof.

52. The method of claim 51, wherein the metabolic cataract comprises diabetic metabolic cataract;
the traumatic cataract comprises surgery-related cataract; and/or

53. The method of claim 49, wherein, the method also is used for (b) inhibiting and/or reversing lens protein aggregation; and/or (c) preventing and/or treating a disease related to lens protein aggregation.

54. The method of claim 49, wherein, the administration route is ocular administration.

55. A compound of formula II, or an optical isomer, racemate, solvate, pharmaceutically acceptable salt, prodrug, or deuterated compound thereof, 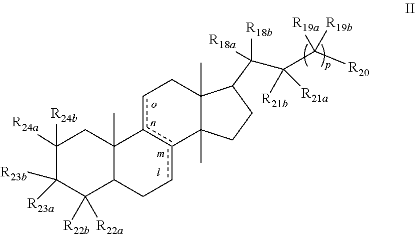
wherein,
p is 0, 1 or 2;
R18a and R18b are each independently hydrogen, substituted or unsubstituted C1-C4 alkyl, or —COOH;
R19a and R19b are each independently hydrogen, substituted or unsubstituted C1-C4 alkyl, or halogen;
R20 is a substituted or unsubstituted C1-C6 acyl, substituted or unsubstituted C1-C6 alkyl, substituted or unsubstituted glycosyl-O—(C1-C6 alkyl)-, substituted or unsubstituted oximido, substituted or unsubstituted C3-C6 heterocycloalkyl, or substituted or unsubstituted C2-C6 alkenyl;
R21a and R21b are each independently hydrogen, hydroxy, thiol, or substituted or unsubstituted C1-C4 alkyl;
R22a and R22b are each independently hydrogen, or substituted or unsubstituted C1-C4 alkyl;
R23a and R23b are each independently hydrogen, hydroxy, thiol, glycosyl, or substituted or unsubstituted C1-C alkoxy;
R24a and R24b are each independently hydrogen, hydroxy, or halogen;
the “substituted” means that one or more hydrogen atoms of the group are substituted by a group selected from halogen, —OH, —SH, —COOH, ═O, —CHO, C1-C4 alkyl, C3-C6 cycloalkyl, amino, glycosyl.

56. The compound of formula II of claim 55, wherein the compound of formula II has one or more features selected from the group consisting of:
R18a and R18b are each independently —COOH, or methyl;
R19a and R19b are each independently hydrogen or halogen;
R20 is hydroxybutyryl, hydroxypropyl, oximido, methoxypropyl, dimethyl epoxyethyl, HOOC—CH═CH—, or hexa-monosaccharide propyl;
R21a and R21b are each independently hydrogen, hydroxy, or thiol;
R22a and R22b are each independently methyl;
R23a and R23b are each independently hydrogen, hydroxy, thiol or hexa-monosaccharide group; and
R24a is hydrogen and R24b is fluorine.

57. The compound of formula II of claim 55, wherein the compound of formula II is selected from the group consisting of: 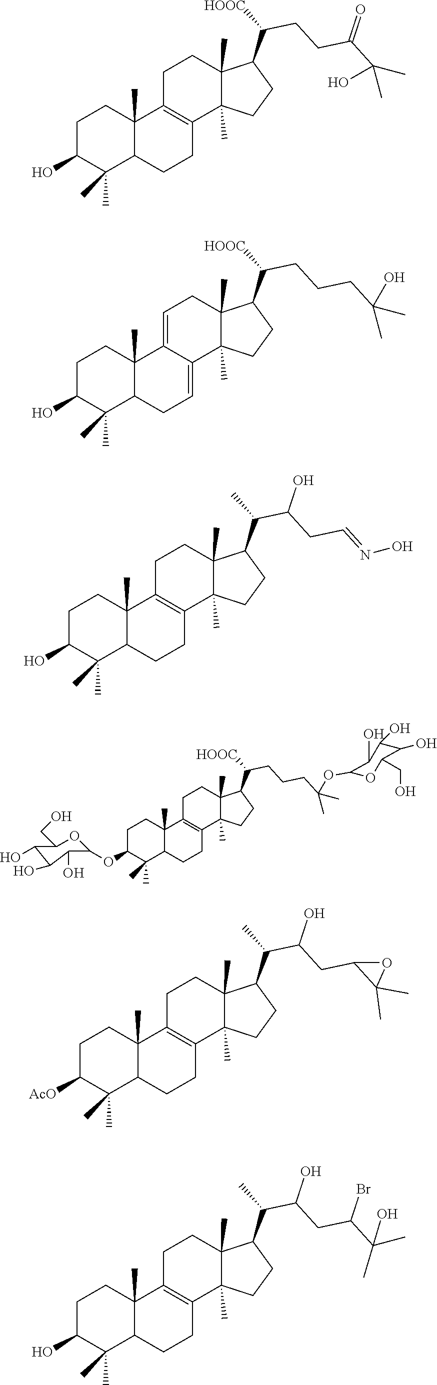 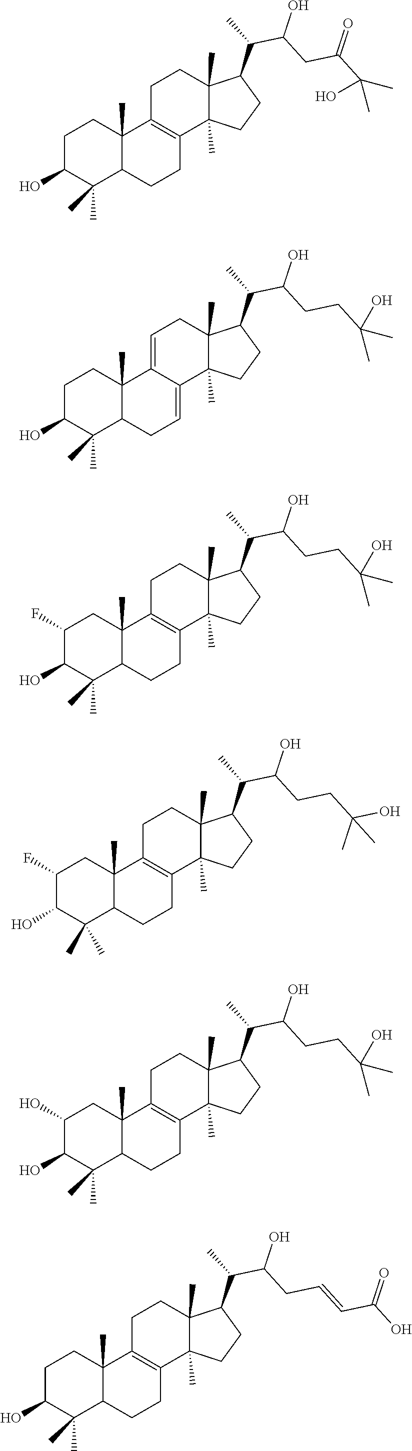 [chemical structure]
